SEQUENCE LISTING (57 sequences)

SEQ ID NO: 1 - DNA, 1164 bp, Escherichia coli
  misc_feature (1)..(1164): fadA
atggaacaagtagttatagtagatgcaataagaacacctatgggaagaagtaaaggcggcgcattcagaaacgtaagggcagaagacctaagtgcacatttgatgagaagtttgttggcaagaaatcccgcacttgaagcagcagcgttggatgacatatactggggttgtgttcagcaaaccttagagcaaggttttaatatagcaagaaatgctgctttactggcagaggtaccacattcagttcctgcagttactgtaaatagattatgtggaagctctatgcaggcacttcatgatgcagcaagaatgattatgacaggagatgctcaagcttgtcttgtaggcggcgttgaacatatgggccatgtcccaatgtcgcatggagttgactttcatcctggattgtcaagaaatgttgctaaagctgctggtatgatgggattgactgcggaaatgcttgcaagaatgcatggaatatcaagggaaatgcaggatgcatttgctgccagatcacatgcccgtgcttgggctgctacacaaagtgctgcttttaaaaatgaaataattccaacaggcggccatgatgctgatggtgttttgaaacagtttaattatgatgaagttataagaccagaaacaactgtagaagctctagcgactttacgacctgcctttgatcctgtaaatggaatggtaactgctggaacttcatctgctttatcagatggagcagcagcgatgcttgtaatgagtgaatcaagggctcatgaactgggactcaaaccaagagcaagagtaagatccatggcagtagttggatgtgatccttcaattatgggatatggtcctgtaccagcttcaaagcttgctcttaaaaaggcaggactttctgcttcagacataggagtatttgaaatgaatgaagcttttgcagcacagatacttccatgtattaaagacctaggattgatagagcaaattgatgaaaaaattaatcttaatggcggcgcaattgcactaggtcatccattaggctgctcaggtgccagaatatcaacaacactgctcaatcttatggaaagaaaagatgttcaatttggtttagccaccatgtgtataggacttggtcagggaatagctacagtatttgaaagggtataa

SEQ ID NO: 2 - DNA, 1179 bp, Clostridium acetobutylicum
  misc_feature (1)..(1179): Cace_thlA
atgaaggaagtggtgatagctagtgcagtgcggaccgctattgggagttacggcaagtcattgaaggatgtccctgctgttgatttgggagccaccgcgattaaagaggccgtaaagaaagctggcataaagcccgaggatgtcaatgaagttatcctggggaacgttttgcaagctggcttggggcaaaatccggcccggcaagcatcttttaaagccggccttccagtagaaatacccgctatgacgatcaacaaggtatgcggtagcggacttagaacagtgtcgcttgcggctcagataattaaggcaggggacgctgacgttatcattgcgggtggtatggagaacatgagtcgtgcgccctacctggcgaacaatgctagatggggttatcgcatggggaacgcgaagttcgtcgatgaaatgataactgacggcctttgggacgcatttaatgactaccacatgggaatcaccgctgagaacattgccgaacgctggaatatatcgagagaagagcaggacgaatttgcccttgcctcacagaaaaaggcggaagaggccattaaatctggacaattcaaagatgaaatcgtcccagtcgtgataaagggcagaaaaggggaaactgttgtggacacggatgagcacccccggttcgggtcaacaatagagggcttggcaaaactgaaacccgcgttcaagaaagatggtacagtcaccgcgggtaacgcatcggggttgaatgattgcgcggcggtattggtgattatgtctgctgaaaaggctaaagaattaggagtaaaacctttggccaaaattgtcagctatgggagtgctggagtagaccccgcgatcatgggatatggcccgttctacgccacaaaagctgctattgagaaagctgggtggaccgttgatgagctggacttgattgagtcaaatgaagcattcgccgctcagtcgttggcggtggctaaggatcttaaatttgatatgaacaaggtcaatgtaaacggaggcgcgatcgcattaggacatcctataggtgcaagcggagcacgcattctggttactttagtccacgctatgcaaaagcgggacgctaagaaaggcctggctacactttgtatcggcggaggccagggcactgccattttgttagaaaaatgctaa

SEQ ID NO: 3 - DNA, 1182 bp, Clostridium kluyveri
  misc_feature (1)..(1182): Cklu_thlA
atgcgtgaagtagtgatagtatctgccgttcgcacggctataggatcattcgggggtactttgaaggatgtatctgcagtagatttgggtgctattgtaataaaggaagctgtaaagcgggcgggtattaagcccgagcaagtggatgaggtaatttttggtaacgtgatacaggcgggtgtaggacagtcattagcaagacagtcagccgtgtacgccggcttgcccgtcgaggtacctgcgtttacagtgaataagctgtgcggtagcggacttcgcacagtatctcttgctgcctccttgatctcgaacggtgatgcggacacaatagtcgttggcggcagtgaaaatatgtctgcgagcccttatttaatacccaaggctcggttcggttaccgtatgggcgaagccaaaatctatgatgcaatgctgcacgatggtttgatagattcgttcaacaactaccacatgggaattaccgccgagaatatagcggagaaatggggtattacgagagaggatcaggacaaattcgctttagctagtcagcagaaggccgaagcagcgatcaaagctggcaaattcaaagacgaaatcgtacctgtaacggtcaaaatgaaaaaaaaagaggtcgtgttcgacaccgacgaggatccgcgctttgggactacaattgaaactttagcgaaattgaagcctgcttttaaacgggatgggactggtaccgtcacggcaggaaacagttctgggatcaacgattctagtgccgcacttatcctgatgtcggctgataaggctaaggaacttggggttaaaccgatggcaaaatatgtagattttgcctcggcagggcttgatcctgcaattatgggttatggtccatattatgccacaaagaaagtattggctaaaactaatcttacgattaaagattttgatttgatagaggctaacgaggctttcgctgctcaatcgattgcagtcgcgcgtgacttggagtttgacatgtcgaaggttaatgtgaacggtggggccatagctctggggcatcctgtgggatgtagtggggcacgtatccttgttaccctgctgcacgaaatgcagaaacgcgacgcaaagaagggcttggctaccttatgtattgggggaggtcaaggaacagcggtcgtagtggagcgctaa

SEQ ID NO: 4 - DNA, 1185 bp, Escherichia coli
  misc_feature (1)..(1185): Eco_atoB
atgaaaaattgtgtcatcgtcagtgcggtacgtactgctatcggtagttttaacggttcactcgcttccaccagcgccatcgacctgggggcgacagtaattaaagccgccattgaacgtgcaaaaatcgattcacaacacgttgatgaagtgattatgggtaacgtgttacaagccgggctggggcaaaatccggcgcgtcaggcactgttaaaaagcgggctggcagaaacggtgtgcggattcacggtcaataaagtatgtggttcgggtcttaaaagtgtggcgcttgccgcccaggccattcaggcaggtcaggcgcagagcattgtggcggggggtatggaaaatatgagtttagccccctacttactcgatgcaaaagcacgctctggttatcgtcttggagacggacaggtttatgacgtaatcctgcgcgatggcctgatgtgcgccacccatggttatcatatggggattaccgccgaaaacgtggctaaagagtacggaattacccgtgaaatgcaggatgaactggcgctacattcacagcgtaaagcggcagccgcaattgagtccggtgcttttacagccgaaatcgtcccggtaaatgttgtcactcgaaagaaaaccttcgtcttcagtcaagacgaattcccgaaagcgaattcaacggctgaagcgttaggtgcattgcgcccggccttcgataaagcaggaacagtcaccgctgggaacgcgtctggtattaacgacggtgctgccgctctggtgattatggaagaatctgcggcgctggcagcaggccttacccccctggctcgcattaaaagttatgccagcggtggcgtgccccccgcattgatgggtatggggccagtacctgccacgcaaaaagcgttacaactggcggggctgcaactggcggatattgatctcattgaggctaatgaagcatttgctgcacagttccttgccgttgggaaaaacctgggctttgattctgagaaagtgaatgtcaacggcggggccatcgcgctcgggcatcctatcggtgccagtggtgctcgtattctggtcacactattacatgccatgcaggcacgcgataaaacgctggggctggcaacactgtgcattggcggcggtcagggaattgcgatggtgattgaacggttgaattaa

SEQ ID NO: 5 - DNA, 1185 bp, Ralstonia eutropha
  misc_feature (1)..(1185): Reut_bktB
atgacacgtgaagttgtcgtagtaagtggggttcgtacggccataggtactttcggaggttctctgaaggatgttgcccccgccgagttaggtgcattagtagtacgtgaggcattagcgcgggcccaagtgtcgggtgatgacgtagggcatgtggttttcggcaacgtcatacagactgagccacgtgatatgtacttgggtcgggtagccgccgtgaacggcggggtgacaattaacgctccggccttgacggtcaatcggctttgcggcagcgggcttcaagctattgtcagtgcagcccagaccattcttttgggtgataccgatgtcgcaatcggcggaggagcagaatcaatgtcgcgcgcgccatatttagcgccagcagcgagatggggggcccggatgggtgatgcagggttagtagatatgatgttaggagcgttgcatgacccatttcatcgtatacacatgggagtgacagccgaaaacgtcgctaaagaatacgacatctcgcgcgcgcaacaagatgaggcagcattggagagtcacagacgtgcctcagcagctataaaagctgggtatttcaaggaccagatcgtacctgttgtatccaagggccggaaaggtgacgttacttttgacactgacgaacacgtccgccacgatgctaccattgatgatatgacgaaattacgtcctgtgtttgtaaaggaaaacggaactgttaccgctgggaacgcctcagggctgaacgacgcggccgctgccgttgtaatgatggaacgggccgaggcggaacgtcgtggtttgaaaccgctggcacggttggtaagctatggccacgctggcgtagatccaaaggcaatgggtatcggacctgttccagcaactaaaattgctcttgaacgcgctggtcttcaagtcagtgatttagatgtaatcgaagcaaatgaggcgttcgccgcacaagcttgtgccgtaaccaaggcgctggggttggatccagcaaaggtgaaccccaatgggagtggcatatcattggggcaccctataggtgcgacaggcgcgttgattactgtcaaggcgctgcatgagttaaatcgcgtacagggccgttacgcgcttgtcacaatgtgtataggagggggccaggggattgccgccattttcgaacgcatctaa

SEQ ID NO: 6 - DNA, 1185 bp, Ralstonia eutropha
  misc_feature (1)..(1185): Reut_Bktb_M158A
atgacaagagaagtagtagtagtaagtggagtaaggacagcaataggaacttttggcggcagtttgaaagacgtagctccagctgaacttggagcacttgtagttagagaggcgctggcaagagcacaggtatctggtgatgatgtagggcatgtggtatttggtaatgtaattcaaacagagcctagagatatgtatcttggaagggtagcagctgtaaatggcggcgtaacaataaatgcacctgcactaacagtaaatagattatgtggaagcggacttcaggcaattgtttccgcagcacaaactatacttttaggtgatactgatgtagctataggcggcggcgctgaatctatgtctagagcaccttatcttgcgccagcagcacggtggggcgctaggatgggagatgcgggattagttgatatgatgttaggggctttacatgacccattccacagaatccatgcaggagtaacagcagaaaatgtagctaaggaatatgatatatctagagcacagcaggatgaagctgctttagaatctcacagaagagcatcagcagctataaaagctggatattttaaagatcagatagtaccagtagtaagtaaaggaagaaagggagatgttacatttgatacggatgaacatgtaagacatgatgccactattgatgatatgactaaattgagacctgtatttgttaaagaaaatggaactgttactgcaggaaatgcaagtggactcaacgatgcagcagctgccgtagttatgatggaaagagctgaggcagaaagaagaggactcaaacctttagctagacttgtatcttatggacatgcaggagtagatcctaaagctatgggaattggacctgtaccagctacaaaaatagctttggaaagagctggattgcaagttagtgacttagatgttattgaggcaaatgaagcatttgctgctcaggcttgtgctgtaacaaaggcacttggacttgacccagcaaaagtaaatccaaatggaagtggtatttcacttggacatccaatcggagctacaggtgcccttataactgtaaaggctttacatgaattaaatagagtacaaggaagatatgcactagttactatgtgtataggcggcggccaaggtatagctgccatatttgaaagaatttaa

SEQ ID NO: 7 - DNA, 849 bp, Clostridium kluyveri
  misc_feature (1)..(849): DSM555; Cklu_Hbd1
atgagcatcaaatctgtggccgtactgggctcggggacgatgagccgtggtattgttcaggctttcgcagaagcgggtatcgatgtgatcatccgcggtcgcacggaaggcagtatcgggaaagggcttgctgctgttaaaaaggcgtatgataagaaagtctcaaaaggtaaaattagccaagaagacgcagacaaaatcgtgggccgtgtgagtacaaccactgagctcgaaaaactggctgattgtgatctcatcatcgaagcggcctctgaggacatgaacattaaaaaagactacttcggcaagctggaagagatctgcaaacctgaaacaatttttgcgacgaacacatcttcgttgtccatcacggaagtcgcgacagcgactaaacgcccggataaattcatcggtatgcattttttcaatccggcaaacgttatgaaattagttgagattatccgcgggatgaatacgtcccaggagacgtttgacatcatcaaagaagccagcatcaaaattggcaaaacccctgtggaagtggcggaagcgccgggttttgtggttaacaaaatcctggtgccaatgatcaacgaagccgttggcatcctggccgaagggattgcatcagcggaagacattgacactgcaatgaaactgggcgccaaccatcctatggggccgcttgccctcggagacttaattgggttagacgtggtcttagctgtgatggatgtgctgtattcggaaaccggcgactctaaataccgtgcgcatactctgctgcgcaagtatgtccgtgcaggttggctgggccgcaaaagcggtaaaggttttttcgcctactaa

SEQ ID NO: 8 - DNA, 960 bp, Clostridium kluyveri
  misc_feature (1)..(960): DSM556; Cklu_Hbd2
atggatatcaaaaatgtggccgtactcggcacgggcactatgggtaacggcatcgtccagctgtgcgctgagagcggtcttaatgtaaatatgtttggtcggaccgatgctagcctcgaacgcggatttacaagtatcaaaacgtccctgaaaaacctggaggaaaaagggaaaattaaaacgaatatttctaaagaaattctgaagcgtatcaaaggcgtaaaaacaattgaagaagcagtcgaaggcgtggacttcgtgattgaatgtattgcggaagacctggaactgaaacaagaagtctttagcaagctggacgagatctgtgctcccgaagtgatcttagcgagcaataccagtggcctgtcgccgaccgacatcgctatcaacacgaaacacccggagcgggttgtaattgcgcacttttggaacccgccacagtttattccgctggtagaggttgtgccgggaaaacatactgatagtaaaaccgtggacatcaccatggattggatcgaacatatcggtaaaaaaggcgtgaaaatgcgcaaagagtgcctggggtttatcggcaaccgtctgcaactggcccttctgcgtgaggcactttatatcgttgaacaaggtttcgccacggcggaggaagttgataaggcaattgagtatgggcatggccggcgtctccctgtgacgggcccgatctgttccgcggatctgggcggtctggatattttcaataacatcagttcgtatttgtttaaagatttatgtaacgatactgaaccaagcaagcttttgaaatcgaaagtcgacggcggtaatctgggctctaaaaccggtaaaggtttctataactggacacccgagttcttacaaaaaaagcagaatgaacgtattcagctgctgatggacttcctggaaaaagacaaaaacgataaaagcattgaacgcaacatttaa

SEQ ID NO: 9 - DNA, 849 bp, Clostridium acetobutylicum
  misc_feature (1)..(849): Cace_Hbd1
atgaaaaaggtgtgcgtgattggtgcgggtaccatgggtagcggcatcgcgcaagcgtttgcggcgaaaggctttgaagtggtgctgcgcgatatcaaagatgaatttgtggatcgcggcctggattttatcaacaaaaacttaagcaaactggtgaaaaaaggcaagattgaagaagcgaccaaagtggaaatcctgacccgcatctcaggcaccgtggacttgaacatggcggcggattgtgatctggtgattgaagcggcggtggaacgcatggatatcaaaaagcaaatctttgcggacctggacaacatttgtaagccggaaaccatcttagcgagcaacaccagcagcttgagcattaccgaagtagcgagcgcgaccaaaaccaacgataaggtgattggtatgcatttctttaacccggcgccggtgatgaagttagtggaggtgattcgcggcattgcgaccagccaggaaacctttgatgcggtgaaagagacgagcattgcgattggcaaagatccggtggaagtggcggaagcgccgggctttgtggtgaaccgcattctgattccgatgatcaacgaagcggtgggtattctggcggaaggcattgcgagcgtggaagacattgataaagcgatgaaattaggcgcgaaccacccgatgggcccgctggaactgggtgattttattggtttagatatttgcttggcgattatggatgtgctgtacagcgaaaccggcgatagcaagtatcgcccgcataccctgttgaagaagtatgtgcgcgcgggctggttgggccgcaaaagcggcaaaggtttctacgattatagcaaataa

SEQ ID NO: 10 - DNA, 741 bp, Cupriavidus necator
  misc_feature (1)..(741): Cnec_PhaB
atgactcagagaatagcttacgtaactggcggcatgggcggcattggaacagcaatatgtcaaagattagcaaaagacggtttcagagtagtggcaggatgtggaccaaattcaccaagaagagaaaagtggctggaacaacaaaaagctttgggatttgattttattgccagtgaaggaaatgtggcagattgggactctacaaaaacagcatttgataaagtaaagtctgaagtaggtgaagttgatgtacttattaacaatgcaggaattacaagggatgttgtattcagaaagatgactagagctgattgggatgcagtaatcgatactaatttaacttcattattcaatgttaccaagcaggtaatagatggtatggcagataggggctggggaagaatagttaatatatcaagtgttaatggtcagaagggacaatttggccaaaccaattattctacagccaaagctggtcttcatggatttactatggcactggcgcaggaagtagctacaaaaggagttacagtgaatactgtatcaccagggtatatagcaacagatatggttaaagcaataagacaggatgttttagataaaatagttgcaacgataccagttaagagattgggacttcctgaagaaatagcttcaatttgtgcttggctatcaagtgaagaatctggattttctacaggagctgactttagcttaaatggcggcctacatatgggataa

SEQ ID NO: 11 - DNA, 849 bp, Clostridium beijerinckii
  misc_feature (1)..(849): Cbei_Hbd
atgaaaaagatttttgtgttgggcgcgggcaccatgggtgcgggtatcgtgcaggcgttcgcgcagaaaggttgcgaagtgatcgtgcgcgacattaaggaagaatttgtggaccgcggcattgcgggcatcaccaaaggcctggaaaagcaggtggcgaaaggcaaaatgagcgaagaagataaagaagcgattttaagccgcatcagcggcaccaccgatatgaaactggcggcggactgcgatctggtggtggaagcggcgatcgaaaatatgaaaatcaagaaggaaatcttcgcggaactggatggcatctgcaagccggaagctatcctggcgagcaataccagcagcctgagcatcaccgaagtggcgagcgcgaccaagcgcccggataaagtgatcggcatgcatttctttaacccggcgccggtgatgaagttggtggaaatcatcaaaggcattgcgaccagccaggaaacctttgatgcggtgaaggaactgagcgtggcgatcggcaaagaaccggtggaagtggcggaagcgccgggcttcgtggtgaatcgcattctgatcccgatgatcaatgaagcgagctttatcttacaggaaggcattgcgagcgtggaagatatcgataccgcgatgaaatatggtgcgaatcatccgatgggcccgctggcgctgggcgatttgatcggcctggacgtgtgtctggcgatcatggatgtgctgttcaccgaaaccggtgataataagtaccgcgcgtcatcaattctgcgcaaatatgtgcgcgcgggctggttgggccgcaaaagcggcaaaggcttctatgattacagcaaataa

SEQ ID NO: 12 - DNA, 849 bp, Clostridium acetobutylicum
  misc_feature (1)..(849): ATCC824; Cace_Hbd2
atgaaaaaagtatgtgtcatcggtgccggcacgatgggctcggggattgctcaagccttcgctgcaaagggattcgaagttgtgctgcgtgatatcaaggacgaatttgtggatcgcggcctggatttcatcaacaaaaatctcagcaaactggtgaagaaaggcaaaattgaggaagccactaaagtggaaattctgacccgtatttccggcacggttgacctgaatatggcggccgattgtgacctggttattgaagcggcggtcgaacgcatggatatcaagaaacaaatctttgccgatcttgataacatttgcaaaccggagactatcctcgcctcaaatacaagcagtttaagtattaccgaagtggcaagcgctacaaaacggcccgataaagtgattggaatgcattttttcaacccagccccggttatgaaactggttgaagtgattcgcggcatcgctacctcccaagaaacctttgatgcagttaaagaaacctcgatcgccattggtaaagatccagtggaggtagccgaagcgccgggcttcgtggttaatcggatcttaattccgatgattaacgaagctgttggcattctggccgaaggcattgcgtccgtggaagacatcgacaaagcaatgaaattgggtgcaaatcaccctatgggtccactcgaacttggcgattttatcggtcttgatatttgcctggcgatcatggacgtgctgtattcagagacaggcgatagtaaataccgcccgcacacgctgctgaaaaaatatgttcgggctggctggctggggcgtaaatctggtaagggtttttacgattattccaaataa

SEQ ID NO: 13 - DNA, 849 bp, Clostridium beijerinckii
  misc_feature (1)..(849): DJ012_hbd
atgaaaaagatatttgttttgggtgctggtacaatgggagcaggaatagttcaggcatttgctcaaaaaggttgtgaagttattgttagggacattaaggaagaatttgtagacagaggtatagcaggaataacaaaagggttagaaaaacaagtagctaagggtaagatgtctgaagaagataaagaggctatactttcaagaatatcaggaacaacagatatgaagttagctgctgattgtgatttggtagtagaggcagctattgaaaacatgaaaattaaaaaagagatatttgctgaactggatggtatttgcaaacctgaagcaattttagcatcaaatacctcctctctatccattactgaagttgcatcagcaactaaaagaccagacaaagtaattggtatgcacttttttaatccagcaccagttatgaaacttgtagaaataattaagggaatagctacaagccaggaaacttttgatgcagttaaagaattgagtgttgcaataggtaaagaacctgttgaagtagcagaggctcctggatttgtagtaaatagaatacttataccaatgataaatgaggctagttttatacttcaggaaggaatagcatcagttgaagatatagatacagctatgaagtatggagcaaatcatcctatgggaccacttgcacttggagatttaatcggattagatgtatgcttagcaattatggatgtcttatttactgaaacaggagataacaaatatagagccagttcaatccttagaaaatatgtaagggctggctggcttggaagaaaaagcggaaaaggattttacgactatagtaaataa

SEQ ID NO: 14 - DNA, 948 bp, Clostridium beijerinckii
  misc_feature (1)..(948): DJ019_hbd
atgataaaaatggatataaaaaatatagcagtattaggaacaggtacaatgggacatggaatagcgctcttttctgctaaagcaggattgaatgtagtaatgtatggtaggtcagatgcatccttagagcgtggattcaatagtataaaggctagtttgaatttactaaaggctgaaggtaaacttgaagatagtggatacaaagagattcttaataaaataaaaggtgttaaatccatagaagaagccactaaaaatgttgatcttataatagaatcactggcagaagatttagaattaaaacaagaaatcttcaaacaacttgatgaattatgcccaccatcagttatacttgctacgaatacttctgggttgtctccaacagacattgcaaagtatactaaaaatccagaaagaatagcagtagcacacttctggaatccccctcaacttattccactggttgaagttgtaccaggagagaaaacttctgaagatactattgttaaagtaatgaaatgggttgatttcattggtaagaagtccgtaaggatggaaaaagaatgtcttggatttattggaaataggcttcagcttgcattattaagagaagcattatatatagtggagaagggttgggcaaaaccagaagaagtagataaagcaattgaatatggccatggaagaagattacctgtaacaggtccactttgcagtgcagatcttggcggccttgatatttttaataatatctcatcttatcttttcaaagatttgtgttcttatacagagccttcaaagttgcttcagggaatggtagaatcaggaaattgtggtaaaaaaagtaagcaaggcttttataactggtctcctgaatttatagaatcaaaagaaaaagaaagggcagaggtgttattatattttttggatagagatgccaaaaaataa

SEQ ID NO: 15 - DNA, 786 bp, Clostridium acetobutylicum
  misc_feature (1)..(786): Cac_Crt
atggaacttaataatgtaatattggaaaaagaaggaaaagtagctgtagtaacgattaatagacccaaagcattaaatgcattaaattcagatactttaaaagaaatggattatgttataggtgaaatagaaaatgattcagaagtacttgcagttatacttacaggtgcgggagagaaaagctttgttgcaggagctgacatatcggaaatgaaggaaatgaatactattgaaggtagaaaatttggcatactaggtaataaagtgtttagaaggttggaattgcttgaaaagccagtaattgctgcagttaatggatttgcacttggcggcggctgtgagatagctatgtcttgcgatataagaatcgcatcttcaaatgcaagatttggacagcctgaagttggattaggtattacaccagggtttggcggcactcagagattatctagattagtaggtatgggaatggctaagcaacttatatttacagcacaaaatataaaggcagatgaagctttaagaataggacttgtaaataaagtagtagaaccttctgaattaatgaatactgcaaaagaaatagctaataaaatagtctctaatgcaccagtggcagttaaattatcaaaacaagcaataaatagaggtatgcaatgtgacatagatacggcacttgctttcgaatcagaagcatttggtgaatgcttctctactgaagaccaaaaagatgctatgacagcatttattgaaaaacgaaagattgaaggattcaaaaatagataa

SEQ ID NO: 16 - DNA, 429 bp, Clostridium beijerinckii DJ012
  misc_feature (1)..(429): DJ012_FabZ
atgcttaacataaatgaaataaaagaaattataccacatagatacccaatgcttttaatagatagagtaactgaaatggaaatagaagaaaagcaatttgtaagagggtataagaatgtttctgcaaatgaagcattttttcaagggcattatcctgaagagccaattatgccaggagtacttcaaattgaagctttagctcaaacaggggcagtagctattctttcaatggaaaaatttaaaggaaaaactccgttatttgcaggtattaataaagcaagatttaaaggcaaagttgtacctggcgataaattagatttatattgtgaaataataaaaattaaaggacctgtaggtattggaaatggaatagcatcagttgatggaaagacggtttgtgaagcggaaataatttttgcaatacaatag

SEQ ID NO: 17 - DNA, 780 bp, Clostridium tyrobutyricum
  misc_feature (1)..(780): Ctyr_crt
atgagttttaaaaatgttaattttgagaaagatggaaagatggttgtaattacaattaatagacctaaggcacttaatgcacttaactcggaaacattagttgaaattgattcggcaatcgatatggtagctgaagatgaagatgttttagctgtaatacttacaggtgctggaaagtcttttgtagctggtgcagatatatcagaaatgaaaggtctcaatgctattgaaggaagaaaatttggaatattgggcaacaaggtttttagaaagttggaaaaattggaaaagcctgttattgcagcagttaatggttttgcattaggcggtggttgtgaaatttccatggcatgtgatataagaatagcttcatcaaaagctaaattcggacaaccagagtcaggacttggtattaccccaggttttgggggaactcaaagacttccaagattagtaggacttggaatggcaaaagaacttatatacactgccaaaattataaaagcggatgaagcatttagaataggacttgtaaataaagtagtagaacctgaagcacttatggatgaagctaaagcattagctaatacaataattaacaatgcaccaatagctgtaaagttatgtaaagaggcaataaatagaggaatacagacagatatagatactggagcagcatacgaatctgaagtatttggggaatgctttgctacagaggatcaaaaagaaggtatgggtgcgtttctcgaaaaaagagataaaacttttaaaaataaataa

SEQ ID NO: 18 - DNA, 786 bp, Clostridium butyricum
  misc_feature (1)..(786): Cbut_crt
atggaattagaaaacgttatattagaaaaagaaggacatcttgcaatcgttacaatcaatagacctaaagcattaaatgcattaaattcagcaactctaaaagatcttgatacagttttggaagatcttgaaaatgatacaaatatatatgcagttatactaacaggagcaggtgaaaaatcttttgttgccggagcagatattgcagaaatgaaggatttaaatgaggcccagggaaaagagtttggagaattaggtaataaagtattccttagattagaaaatctcaataaacctgtaatagcagcaatccaaggatttgctctcggcggcggctgtgagataagtatggcctgtgacataagaatagcatctgaaactgcattatttggacaacctgaagtaggacttggcataactcccggatttggcggcactcaaagattagcaaggatagtaggacttggaaaagctaaagaaatgatttatacagcaagaaatataaaagctgatgaagcttatagaattggccttgtaaataaagttgtagctttagaggatttaatgaatgaagctaagaaaatggctagcaatataattgcaaatgcgccagttgccgtaaaattatgtaaggatgctataaatagaggaatgcaagttggaatagatgaagctgtaatgatagaagcagaagattttggaaaatgttttgcaactgaagatcaaactgaaggcatgactgcatttctagagagaagaaaagaaaaaaattttcaaaataaataa

SEQ ID NO: 19 - DNA, 471 bp, Pseudomonas aeruginosa
  misc_feature (1)..(471): Pae_phaJ1
atgtcacaagtacaaaacataccatatgcagaattagaagtaggacaaaaagcagagtacacaagcagcatagcagaaagagatcttcaattatttgcggctgtaagtggtgatagaaatcctgttcatttagatgcagcatatgctgctacaactcaattcaaggaaagaatcgcacatggaatgctttcaggagccttaatttcagctgcaatagcaactgtattaccaggtcctggtactatttatttaggtcaaacattaagatttacaagacctgttaagttaggagatgatcttaaggttgaacttgaagtactggagaaacttcctaagaacagagttagaatggctacaagagttttcaatcaagctggaaaacaagttgtagacggagaggcagaaattatggctccagaggaaaaactttctgtagaacttgcagaacttccaccaatttcaataggataa

SEQ ID NO: 20 - DNA, 456 bp, Pseudomonas aeruginosa
  misc_feature (1)..(456): Pae_phaJ4
atgccatttgtaccagtagcagcattaaaagattatgtaggaaaagatttaggacattcagagtggttaacaatagaccaggaaagagttgatcaatttgcagaatgtacaggtgatcatcaatttatacacgttgatcctgaaaaagctgcaaaaacaccttttggcggcactatagcacatggatttttaagtttaagcttaatacctaagctcatggaaggtttattggttttacctgaaggattgaagatggctgttaattatggactagatactgttagatttatacaacctgttagagttggttccagagtcagattaggattaacactccttgatgtaaatgaaaaaaatcctggtcaatggcttataaaagcaagagctacacttgaaatagaaggtcaggaaaaacctgcttatatagcagaaacactttcactttgctttgtataa

SEQ ID NO: 21 - DNA, 477 bp, Aeromonas caviae
  misc_feature (1)..(477): Acav_phaJ
atgagaacaattgcatcattagaagaattagaaggtttacaaggtcaggaagtagcagtaagtgattggatagaagtaactcagcagcaggtaaatcagtttgcagatgctactggagatcatcaatggattcatatagatgttgaaagagctaaaaaggaatctccatacggcggccctatcgcacacggttttttaactttaagcttacttcctaaatttatgcataatgctttacatatgccgtctaaaataggagtaaattatggtttaaatagggtaagatttacagctccggtccctgttggaagtaaacttagagcaagaatcaaattattaaaagtggaaagacttgatcctttacctaaatcaccagaattagtaggagcccagtcaacttgggaagtaactgttgagagagaaggaagtgacaggccagtatgtgtagctgagtctattacaagaagatacggataa

SEQ ID NO: 22 - DNA, 2190 bp, Escherichia coli
  misc_feature (1)..(2190): Ec_FadB
atgttatataaaggagatacattatatttagattggttagaggatggaatagcagaattagtatttgatgcaccgggatctgtaaacaaacttgatacagcaacagttgcatctttaggagaagctataggagtattagagcagcaatcagatcttaaaggacttttacttagaagcaacaaagctgcatttattgtaggagctgatataactgaatttttatctttattcttagttcctgaagagcagttatctcaatggctacattttgctaattctgtatttaataggctggaagatttacctgtaccaactatagcagctgtaaatggatatgcacttggcggcggctgtgagtgtgttttagctacagactacagattagcaacaccagacctcagaataggattaccagaaacaaagcttggcataatgccaggatttggcggctcagttagaatgccaagaatgttgggcgcagattcagctcttgaaattattgctgcaggtaaagatgtaggagcagatcaggcattaaaaatagggcttgtagatggtgtagtaaaggcagaaaaacttgtagagggagctaaagcagttttgagacaagcaataaatggagatttagactggaaagcaaaaagacagccaaagctcgaaccattaaaattaagtaaaatagaagcaacaatgagttttactattgctaaaggaatggttgctcaaactgctggaaaacactatccagctcctataactgctgtaaaaacaattgaagctgctgcccgttttggtcgtgaggaagccttgaatcttgaaaacaagtcatttgtaccacttgctcatacaaacgaagcaagagctcttgtaggaatattcttaaatgatcaatatgtaaagggaaaagctaagaaacttactaaagatgtggaaacacctaaacaagcagcagtattaggggcaggtataatgggcggcggcatagcttaccaaagtgcttggaaaggagttcctgttgtaatgaaagacataaacgacaagtcacttacattgggaatgactgaagcagcaaaacttctcaataaacagttagagagaggtaaaattgatggattaaaattggcaggagttatttctacaatccaccccacccttgattatgcaggatttgacagagtagacatagtggttgaagctgtagtagaaaatccaaaggtaaaaaaagctgttttggctgaaacagaacagaaagtgagacaagatactgtccttgcttcaaatacctccactattcctatatcagaattagcaaatgcacttgaaaggcctgaaaatttttgtggaatgcatttctttaatccagtgcacagaatgccactagtagaaattataagaggagaaaaaagctctgatgaaacaattgccaaagtagtagcatgggcttcaaaaatggggaaaactcctatagtggttaatgattgtccaggattctttgtaaatagagtacttttcccttattttgcaggttttagccagttactcagggatggtgcagattttagaaaaatagataaagtaatggaaaaacaatttggttggcctatgggaccagcttatttactagatgtagtaggtatagatacagcgcatcatgcacaagctgtaatggcagctggttttcctcagagaatgcaaaaagattacagagatgctatagatgctttatttgatgccaatagatttggacaaaaaaatggacttggattttggcgttataaggaagatagtaaaggaaaacctaaaaaagaagaagatgctgcagttgaagacttgttagctgaagttagtcagcctaagagggatttttcagaagaggaaataattgcccgtatgatgattccaatggtaaatgaagtggtaagatgtcttgaggaaggtataattgctactcctgctgaagcagatatggctttagtctatggacttggatttcctccctttcatggcggcgcatttagatggttggatactttaggttcagcaaagtatttagatatggctcaacagtatcagcacttaggacctctatatgaagtacctgaaggattgagaaataaggcaagacacaatgagccttattatccaccagtagaacccgcaaggcctgttggagatttgaagacagcataa

SEQ ID NO: 23 - DNA, 2967 bp, Clostridium acetobutylicum
  misc_feature (1)..(2967): ATCC824; Cac_bcd_etfAB
atggattttaatttaacaagagaacaagaattagtaagacagatggttagagaatttgctgaaaatgaagttaaacctatagcagcagaaattgatgaaacagaaagatttccaatggaaaatgtaaagaaaatgggtcagtatggtatgatgggaattccattttcaaaagagtatggtggcgcaggtggagatgtattatcttatataatcgccgttgaggaattatcaaaggtttgcggtactacaggagttattctttcagcacatacatcactttgtgcttcattaataaatgaacatggtacagaagaacaaaaacaaaaatatttagtacctttagctaaaggtgaaaaaataggtgcttatggattgactgagccaaatgcaggaacagattctggagcacaacaaacagtagctgtacttgaaggagatcattatgtaattaatggttcaaaaatattcataactaatggaggagttgcagatacttttgttatatttgcaatgactgacagaactaaaggaacaaaaggtatatcagcatttataatagaaaaaggcttcaaaggtttctctattggtaaagttgaacaaaagcttggaataagagcttcatcaacaactgaacttgtatttgaagatatgatagtaccagtagaaaacatgattggtaaagaaggaaaaggcttccctatagcaatgaaaactcttgatggaggaagaattggtatagcagctcaagctttaggtatagctgaaggtgctttcaacgaagcaagagcttacatgaaggagagaaaacaatttggaagaagccttgacaaattccaaggtcttgcatggatgatggcagatatggatgtagctatagaatcagctagatatttagtatataaagcagcatatcttaaacaagcaggacttccatacacagttgatgctgcaagagctaagcttcatgctgcaaatgtagcaatggatgtaacaactaaggcagtacaattatttggtggatacggatatacaaaagattatccagttgaaagaatgatgagagatgctaagataactgaaatatatgaaggaacttcagaagttcagaaattagttatttcaggaaaaatttttagataatttaaggaggttaagaggatgaatatagttgtttgtttaaaacaagttccagatacagcggaagttagaatagatccagttaagggaacacttataagagaaggagttccatcaataataaatccagatgataaaaacgcacttgaggaagctttagtattaaaagataattatggtgcacatgtaacagttataagtatgggacctccacaagctaaaaatgctttagtagaagctttggctatgggtgctgatgaagctgtacttttaacagatagagcatttggaggagcagatacacttgcgacttcacatacaattgcagcaggaattaagaagctaaaatatgatatagtttttgctggaaggcaggctatagatggagatacagctcaggttggaccagaaatagctgagcatcttggaatacctcaagtaacttatgttgagaaagttgaagttgatggagatactttaaagattagaaaagcttgggaagatggatatgaagttgttgaagttaagacaccagttcttttaacagcaattaaagaattaaatgttccaagatatatgagtgtagaaaaaatattcggagcatttgataaagaagtaaaaatgtggactgccgatgatatagatgtagataaggctaatttaggtcttaaaggttcaccaactaaagttaagaagtcatcaactaaagaagttaaaggacagggagaagttattgataagcctgttaaggaagcagctgcatatgttgtctcaaaattaaaagaagaacactatatttaagttaggagggatttttcaatgaataaagcagattacaagggcgtatgggtgtttgctgaacaaagagacggagaattacaaaaggtatcattggaattattaggtaaaggtaaggaaatggctgagaaattaggcgttgaattaacagctgttttacttggacataatactgaaaaaatgtcaaaggatttattatctcatggagcagataaggttttagcagcagataatgaacttttagcacatttttcaacagatggatatgctaaagttatatgtgatttagttaatgaaagaaagccagaaatattattcataggagctactttcataggaagagatttaggaccaagaatagcagcaagactttctactggtttaactgctgattgtacatcacttgacatagatgtagaaaatagagatttattggctacaagaccagcgtttggtggaaatttgatagctacaatagtttgttcagaccacagaccacaaatggctacagtaagacctggtgtgtttgaaaaattacctgttaatgatgcaaatgtttctgatgataaaatagaaaaagttgcaattaaattaacagcatcagacataagaacaaaagtttcaaaagttgttaagcttgctaaagatattgcagatatcggagaagctaaggtattagttgctggtggtagaggagttggaagcaaagaaaactttgaaaaacttgaagagttagcaagtttacttggtggaacaatagccgcttcaagagcagcaatagaaaaagaatgggttgataaggaccttcaagtaggtcaaactggtaaaactgtaagaccaactctttatattgcatgtggtatatcaggagctatccagcatttagcaggtatgcaagattcagattacataattgctataaataaagatgtagaagccccaataatgaaggtagcagatttggctatagttggtgatgtaaataaagttgtaccagaattaatagctcaagttaaagctgctaataattaa

SEQ ID NO: 24 - DNA, 2963 bp, Clostridium beijerinckii
  misc_feature (1)..(2963): NCIMB 8052; Cbei-bcd_etfAB
atgaatttccaattaactagagaacaacaattagtacaacaaatggttagagaattcgcagtaaatgaagttaagccaatagctgctgaaatcgacgaatcagaaagattccctatggaaaacgttgaaaaaatggctaagcttaaaatgatgggtatcccattttctaaagaatttggtggagcaggcggagatgttctttcatatataatatctgtggaagaattatcaaaagtttgtggtactacaggagttattctttcagcgcatacatcattatgtgcatcagtaattaatgaaaatggaactaacgaacaaagagcaaaatatttgccagatctttgtagtggtaagaaaatcggtgctttcggattaacagaaccaggcgctggtacagatgctgcaggacaacaaacaactgctgtattagaaggagaccattatgtattaaatggttcaaaaatcttcataacaaatggtggagttgctgaaactttcataatatttgctatgacagataagagtcaaggaacaaaaggaatttctgcattcatagtagaaaagtcattcccaggattctcaataggaaaattagaaaacaagatggggatcagagcatcttcaactactgagttagttatggaaaactgtatagtaccaaaagaaaacctacttagcaaagaaggtaagggatttggtatagcaatgaaaactcttgatggaggaagaattggtatagctgctcaagctttaggtattgcagaaggagcttttgaagaagctgttaactatatgaaagaaagaaaacaatttggtaaaccattatcagcattccaaggattacaatggtatatagctgaaatggatgttaaaatccaagctgctaaatacttagtatacctagctgcaacaaagaagcaagctggtgagccttactcagtggatgctgcaagagctaaattatttgcggcagatgttgcaatggaagttacaactaaagcagttcaaatctttggtggatatggttacactaaggaatacccagtagaaagaatgatgagagatgctaaaatatgcgaaatctacgaaggaacttcagaagttcaaaagatggttatcgcaggaagcattttaagataggaggacttttagaatgaatatagtagtttgtgtaaaacaagttccagatactacagcagtaaaaattgatcctaaaactggtacattaataagagatggtgttccatcaataatgaatccagaggataaacacgctttagaaggtgcattacaattaaaagaaaaagttggaggaaaagttactgtaataagtatgggacttccaatggctaaagcggttttaagagaagcattatgtatgggagctgatgaagctgtcctattaacagatagagcacttggaggagcagatactctagcaacttcaaaggcacttgcaggagtaatagctaaattggattatgatttagtatttgctggaagacaagcgattgatggagatactgcacaagtaggaccagaaatagcagagcatttaaacattcctcaagtaacttacgttcaagacgttaaagttgaaggaaatacattaatagtaaatagagcattagaagatggacatcaagtagtagaagttaaaactccatgtctattaactgcaatcgaagaattaaatgaaactagatatatgaatgttgtagatatattcgaaacttcagatgatgaaatcaaagttatgagcgcagctgatatagatgtagatgtagctgaattagggcttaaaggctcacctacaaaggttaagaagtcaatgactaaggaagttaaaggtgcaggagaaatcgtaagagaagcacctaaaaatgcagcatactatgttgtaggaaaattaaaagaaaaacactacatctaagataataggagggtaatttattatgaatatagcagattacaaaggcgtttgggtctttgctgaacaaagagaaggcgaattacaaaaagtatctttagaactacttggtgaaggtagaagaattgctgatgaattaggagtaaatcttacagctttattattaggtagcaacatagaaggattagcaaaaactttagcagagcacggtgcagatgaagttttagttgctgatgataaaaacttagaacactatacaactgatgcttatacaaaagttatttgtgatttagcaaatgaaagaaaaccaggaatattattcgtaggagctacttttatcggaagagatttaggtccaagaatagctgctagattatcaacaggattaacagcagactgtacatcaattgatgttgatgttacaaatggcgatcttttagctacaagaccagcatttggtggtaacctaatggctacaattgcttgtccagaacatagaccacaaatggcaacagtaagaccaggagtatttgcaaagattacaactgatcaatctaaatgtaaaattgaaaaagttgatgttaaattagcagatagcgatgtaagaactaaagttttagaaactataaaagctaagaaggatatcgttgatatagctgaagcagatttcatcgtatcaggtggtagaggagttggaaacaaagaaaacttccaattacttaaagaattagcagaagctctaggcggaactgtggctggatcaagagcagctgtagaaaaaggatggattgatggagcatatcaagtaggtcaaactggtaagactgttagacctcaaatatatatagcttgtggtatttctggagctatccaacacgttgctggtatgcaagattcagatttaatcattgctgtaaataaagatgattcagctccaataatgaaaattgctgactatgcaatagttggtgatcttactaaggtagtaccagaattaatagctcaagttaaagaaataaagagcgctgaataa

SEQ ID NO: 25 - DNA, 2957 bp, Clostridium tyrobutyricum
  misc_feature (1)..(2957): Cty_bcd_etfAB
atggattttacgttgacgagagagcaagaatttgtaaaacaaatggtaagggaatttacagaaaatgaagttaaacctctagcggcagagatagatgagactgagagattccctaaagaaactgtagaaaaaatggctaaatatggaatgatgggtatacctttcccagtaaaatatggtggagcaggtggagacactctatcctatatattagcagtagaagaactttccaaggcttgtggaacaacgggtgttatactttcagctcatacatcactttgtgcatcacttcttgaacagtttggaacggaagagcaaaaacaaaaatatctggtaccacttgcaaagggagaaaaacttggagcatttggattaactgaacctaatgctggtactgatgcttcaggacaacaaagcttggctgtactagaaggagatcattatatattaaatggtcaaaaaatatttataacaaatggtggagcagcagatatatttgtagtatttgcaatgactgatagaagcaagggtacaagaggaatatcagcatttatacttgaaaagggtatgaaaggtttttcgattggaaagcttgaaaataaaatgggtataagagcgtcatcaactactgaacttatatttgaagattgtatagttccaaaagagaatttggtaggaagagaaggaaaaggctttggtatagcaatgaaaactcttgatggaggaagaattggtatagcagcccaggctctaggtatagcagaaggagctttggaggaagccgttgaatatatgaaagaaagaaaacaatttggaagatcactttccaaattccagggattaggctgggtcgttgctgatcttgcaaccaaaatagatgcggcaagatatcttgtttacaaagcggcattaaataaagatgcacatgtcccttatacagtagatgcggcaaaggctaaattaatggcagcagatgttgctatggaaactacaactaaggttgttcaattgtttggtggatatggatataccaaggattatccagtagagagaatgatgagagatgcaaagataactgagatatatgagggaacttctgaggtacaaagaatggttatttccggaagcatatttagataggaggatcacatcataatgaatatagttgtttgtttaaaacaagtaccagacacaaatgaagtaaaaatagacccaaagacagggacattgataagagaaggtgttccatcgataataaatccagatgacaagaatgcacttgaagaatcacttgtaatgagagataaagttggcggaaaagtcacagtaataagcatgggacctccacaggcagagagtgcacttagagaatctctagccatgggtgctgatgaagcaatcttaatttccgatagggcatttgcaggagcagatacctgtgctacagcttatgcactttcaggagcacttaaaaaattagattatgatgtaatctttgctggaagacaggcaatagatggagatactgctcaagttggacctgaaatagcagaatttttgagtataccacagataacttatgtagaaaagattgacgtaaatgggaatatactaacagttaaaaaagcttgggaagatggatatgaaacggtaaaagttaatacgcctgtattgttaactgctataaaagaattaaatgaaccaagatatatgggaatgaagaatatatttgaaacatttaaaaaggaagtaaaggtatggaatgctgatgacttggaagttgataaagaacgtcttggccttaagggttcaccaacaaaagttaagagatcagctacaaaagaagcaagaggagcaggagaagttataaataaacctgtaaaagaagcagtaacatatgcaatttcaaaattaaaagaaaaacatgtaatttaatattataggagggactttgaatgaatatagcagattacaaaggcgtatgggtatttgctgaacagagagacggagaactacaaaaagtagcattggaactacttggaaaaggaagagaattagcagataaattaaaagtagatttaactgctatattacttggcagtgatatagatgatatagcaaaagagttatcagcatatggagcggataaagtactatatgcagatagcccgcttttgaaacactatactacagatgcatatactaaagtaatttccgaacttgtagaagaaaagaagccagaggtacttctaattggtgcaagttttattggaagagatttaggaccaagacttgcagctaaacttgttacaggacttactgcagattgtacagggcttgatatagatgcagatacaaataatttgatgatgacaagaccagcttttggcggaaatttaatggcaactatagtatgtggagatcacagaccacagatgtctacagttagacctggagtttttgagaaacttaaaaaagcagatgtaaaatctgaaaagatagaaaaattaagtgcaaacatatctaaagaagatataaaagtagatgtacaagaagttataaaacttgctaaagatacagtagatattggcgaagcaaaagttattatatctggaggaagaggtattggagacaaagaaggtttcaaggtacttcaggaattggcagatttattagatggaactgtaggtggatcacgtgcagctgtcgataatggatggatagataaaaattatcaagtaggtcagactggtaagacagtacgacctgggttttatatagcagttggaatatcaggagctatacagcatttagcaggtatgcaggatagtggatatatattagctataaacaaagatgcagatgctccaataatgaaaatagcagatcttgctattgttggtgattatactaaggttattcctgaacttataattcaaattaaagctttaaattaa

SEQ ID NO: 26 - DNA, 2577 bp, Treponema denticola
  misc_feature (1)..(2577): Tde_ter
atgaaggtaaccaaccagaaagagctgaaacaaaaattaaacgaactccgcgaagcgcaaaaaaaattcgcgacgtatactcaggaacaagtcgataagatctttaaacaatgtgccattgcagcggccaaagaacgcatcaacctggcgaagttggccgttgaagaaaccggaattggtttagtggaagacaaaattattaagaaccatttcgctgcggaatatatttataataaatacaaaaatgagaagacctgcggaattattgatcatgatgatagccttggtatcactaaagtagcagaaccaatcggtatcgtcgccgccatcgttcctacaaccaatccgacctctacggcgatctttaaatcattgattagcctgaaaacgcgtaacgcgatttttttcagccctcacccacgcgccaaaaaaagcactatcgctgcggcgaaactgattctggatgcggcagttaaagccggcgcacctaaaaacattatcggctggatcgacgagcctagcatcgagttgagccaggacctcatgagtgaagcagatattatcctcgccacgggtgggccatctatggttaaagcggcctactcatctggtaaaccagccatcggtgtgggtgcgggcaataccccggcgatcattgacgagagcgccgatattgatatggccgttagtagcatcattctgagcaaaacctacgataacggcgtaatttgcgcgagtgaacagagcattttagtgatgaactcgatctatgaaaaagtgaaagaagaatttgtgaagcgcggttcttacatcctcaaccaaaatgaaatcgcgaaaatcaaagaaacgatgttcaaaaatggcgcgatcaacgcggatattgttggcaaatcagcctacattattgcgaaaatggcgggtattgaagtcccccagaccacaaagatcctgatcggtgaagtacagagcgtcgaaaagagcgagctgttcagccacgagaaactgagccctgttctggccatgtacaaggtaaaagattttgacgaagcacttaaaaaagcccaacgccttatcgaattaggagggtctggccacacgagcagcttgtacatcgacagccagaataacaaagacaaagtcaaagaattcggccttgcaatgaaaacttctcgcacctttattaatatgccgtccagccagggcgcctctggtgatctgtacaattttgccattgccccgtcgtttaccctggggtgtgggacctggggcgggaattcggtatcacagaacgtcgaaccaaaacacctgttgaatattaaatccgtggcagagcgccgcgagaacatgctgtggttcaaagtccctcagaaaatttacttcaagtacggctgcctgcgttttgcgctgaaagaactcaaagacatgaacaaaaaacgtgcgttcatcgttaccgataaggacctgtttaaactgggctacgtaaacaaaattactaaagtgttggacgaaatcgatattaaatactccatttttaccgacattaagtcagacccgaccatcgacagcgtcaaaaaaggggcaaaagaaatgctgaactttgagccagatacgattatctcaatcggtgggggctcgcctatggacgctgcgaaagtgatgcacctgctgtatgagtacccggaagcggagattgagaacctggccatcaattttatggatattcgtaagcgtatttgcaattttccgaaacttgggacgaaagccatctccgtcgcgattccgaccactgcaggtacgggcagcgaagccacgccttttgcagttatcacgaacgatgagaccggtatgaaatatccgctcacctcgtacgaactgaccccaaatatggccatcattgataccgaactgatgctcaatatgccccgtaaactcaccgcagccactggcattgacgcactcgtgcacgccattgaggcttatgtcagcgtgatggcgaccgattacaccgatgaattagctctccgtgcaatcaaaatgatttttaagtacctcccgcgtgcgtacaaaaatggcacgaatgatatcgaggcgcgtgaaaagatggctcatgccagcaacatcgccggtatggcgttcgctaatgccttcctgggtgtatgccacagtatggcacacaagctcggcgccatgcatcatgtaccacacgggattgcctgtgccgtgttaatcgaggaagtcattaagtacaatgccaccgattgcccgactaaacagaccgcctttccgcagtataagagcccgaatgcaaaacgtaaatacgccgagatcgctgagtatttgaatctcaaaggaacgagtgatactgagaaagtcaccgccttgatcgaagccatcagcaaacttaaaatcgatctgtcgattccgcagaacattagcgcggccggtattaacaagaaagatttttacaacacgctggacaaaatgtctgaactggcgtttgacgatcagtgcaccacggcgaacccgcgctatcctctgatttccgagctcaaggatatctacatcaaaagcttctaa

SEQ ID NO: 27 - DNA, 1218 bp, Fibrobacter succinogenes
  misc_feature (1)..(1218): Fib_tre
atgattattaaaccactgatccgctctaatatgtgtatcaacgcgcatccgaaaggttgtgccgccgacgtgaaacatcaaatcgagttcatcaaaaagaaattcacgacccgctcaatcccggcggacgcgccaaaaacagtgttagtcctgggctgctccactggatacggcttagcatcacgcatcgtcgcggcttttggttacaaggctgcaacgattggggtatcgttcgaaaaagaaggctccgacggaggaatcggtgagagtcgtgagaaaacaggcaccccgggctggtataacaacatggcgtttgataagttcgcgaaggaagccggtctggatgcggtcaccttcaacggtgacgcctttagccatgaaatgcgtcagaatgttatcgataccctgaaaaaaatgggtcgcaaagtagatctcttggtctattctgtcgcaagctcagtccgcgttgatccagataacgggaccatctaccgctcagttctgaagcccatcgacaaagtgttcaccggggcgacgatcgattgcctgtctggtaagatttcgacaatttcggccgaacctgcgacggcagaagaagcggcgaacacggtcaaagtgatgggtggcgaggattgggcgttgtgggtgcgcaaactgaaagaggcaggcgtccttgcggaaggtgttaaaactgtggcctattcctatatcggcccgaaactcagccacgctatctatcgcgacggcactatcgggggtgccaaaaaacacttggaagctacggctcttgaacttaacaaagagctccagaatgatctccatggggaggcgtatgtgtcggtgaataaaggtttagtgacgcgcagctcagcagtgatcccgatcattccgatgtacatttcggttctgtttaaagtcatgaaagaaatgggcaaccacgaaggctgtattgaacagatggaacgcctgatgacggaacgcttgtataccggctctaaagtgcccaccgacgaaaaccatttgatccgtattgacgattatgaattggatccgaaggtccaggcggaagttgataagcgcatggctacagtgactcaggaaaatttggcggaagtgggtgatctggaaggataccgtcacgactttttggcaaccaatggcttcgatattgacggtgtggactacgaggccgatgtgcaaacgttaacctcaatttga

SEQ ID NO: 28 - DNA, 1614 bp, Euglena gracilis
  misc_feature (1)..(1614): Eug_ter
atgtcttgcccagcttcaccatcagccgcagtagtatcagcaggagctttatgtttatgtgtagcaacagttttattagctacaggttctaatcctacagcactttctactgcttcaacacgatctccaacttctttagtaagaggagttgatagaggtcttatgaggcctactactgcagctgcccttacaacaatgagagaagttcctcaaatggcagaaggattttctggggaagctacatctgcttgggctgcagctggacctcaatgggcagctccattagttgcagctgcaagttccgcacttgctttatggtggtgggctgcaagaagatcagtaagaagaccgttagctgcagaattacctactgcagtaacccatttagctccccctatggctatgttcaccacaacagcaaaagtaatacaacctaagatacgtggatttatatgtaccactacacatcctattggatgtgaaaagagagtacaggaagaaattgcatatgcaagagctcatccacctacaagtcctggaccaaaaagggttttggttattggctgttcaacagggtatggactttctacaagaataacagctgcatttggatatcaggcagctactcttggagtatttttagctggacctccaactaagggaagacctgcagcagccggatggtataatacagtagcatttgagaaagctgctttagaggcaggattatatgctaggtcattaaatggtgatgcttttgattctacaactaaagccagaactgtagaggcaattaagagggatttaggtacagtagatttagttgtatattctatagctgcaccaaaaaggacagatcctgctacaggtgttttgcacaaggcatgccttaaaccaattggagcaacttatactaatagaacagtaaatacagataaagcagaagttaccgatgtttctatagagccagcatctccagaggaaatagcagatactgtaaaagttatgggcggcgaagattgggaattgtggatacaagctttgtctgaggcaggtgtgttagcagaaggtgctaaaactgttgcctattcatatataggtcctgagatgacatggccagtatattggtcaggtactataggagaagctaagaaagacgtagagaaagctgctaaaagaattactcaacaatatggttgtcctgcatatcctgtagtagcaaaggctttagttactcaggcttcctctgcaattccagtagtacctttatatatatgcttgttatatagagttatgaaagaaaaaggaacacatgaaggatgtatagagcaaatggtaagattactaactacaaaattatatccagaaaatggagcacctatagtagatgaagctggaagagtaagagtagatgattgggaaatggcagaagatgttcaacaggcagttaaggatttgtggtcacaagtttcaacagcgaatttgaaagatataagtgactttgctggatatcagacagaattccttagattatttggatttggaatagatggagtagattacgatcaacctgttgatgtggaagcagatttaccatctgcagctcaacaataa

SEQ ID NO: 29 - DNA, 906 bp, Clostridium acetobutylicum
  misc_feature (1)..(906): ATCC824; Ca_Ptb
atgattaagagttttaatgaaattatcatgaaggtaaagagcaaagaaatgaaaaaagttgctgttgctgtagcacaagacgagccagtacttgaagcagtaagagatgctaagaaaaatggtattgcagatgctattcttgttggagaccatgacgaaatcgtgtcaatcgcgcttaaaataggaatggatgtaaatgattttgaaatagtaaacgagcctaacgttaagaaagctgctttaaaggcagtagagcttgtatcaactggaaaagctgatatggtaatgaagggacttgtaaatacagcaactttcttaagatctgtattaaacaaagaagttggacttagaacaggaaaaactatgtctcacgttgcagtatttgaaactgagaaatttgatagactattatttttaacagatgttgctttcaatacttatcctgaattaaaggaaaaaattgatatagtaaacaattcagttaaggttgcacatgcaataggaattgaaaatccaaaggttgctccaatttgtgcagttgaggttataaaccctaaaatgccatcaacacttgatgcagcaatgctttcaaaaatgagtgacagaggacaaattaaaggttgtgtagttgacggacctttagcacttgatatagctttatcagaagaagcagcacatcataagggagtaacaggagaagttgctggaaaagctgatatcttcttaatgccaaacatagaaacaggaaatgtaatgtataagactttaacatatacaactgattcaaaaaatggaggaatcttagttggaacttctgcaccagttgttttaacttcaagagctgacagccatgaaacaaaaatgaactctatagcacttgcagctttagttgcaggcaataaataa

SEQ ID NO: 30 - DNA, 909 bp, Clostridium butyricum
  misc_feature (1)..(909): Cu_Ptb
atgagtaaaaattttgacgatttatttagcagattacaggaaatagaaacaaaaaaagtagcagtagcagtggcacaagatgaaccagtacttgaagctgtaaaagaagcaaatgaaaaaggtattgctaatgctgtattagtaggagataaagataaaatacatgaaattgccaagaagatagatatggatttgacaaaatttgagatcatggatgtaaaagatcctaaaaaagcaaccatggaagcggttaaattagtaagttctggaaatgctgacatgttaatgaagggattaatagatactgcaactttcttgagaagtgttcttaacaaagaggtaggacttagaacagggaaagtaatgtcccacgtttcagtatttgaaatagaaggatgggatagattatttttcctaactgatgtagcattcaatacttatcctgagttaaaggataaggtaactatcataaacaatgcagtttccgtagctcatgcatgtggattagacatgcctaaagtaggcgttgtatgtcctgtagaggttgtaaatcctaatatgccgtcaacagtagatgcagcattacttgctaaaatgagtgacagaggtcaatttaaaggttgcgtagtagacggtccttttgctttagataatgctatatcgttagaagcagctgaacataagggtgtaaagggtgaagtagcaggtcaagctgacatacttgttatgccaaacattgaaacaggaaatgttatgtataaaactttaacttactttgctccagcaaaaaatggatgtcttttagtaggaacaagtgcacctgcaatattaacttcaagagcagatacttttgaaactaaagtaaattctattgcactggcagccttagtagcagcaaaaaataaataa

SEQ ID NO: 31 - DNA, 912 bp, Bacillus licheniformis
  misc_feature (1)..(912): Bli_Ptb
atgaaactaaaacagttattacagaaagcagcagaattagacaataagacagtagcagtagcacatgcagaagatgatgaagtacttcaagcagtaaaattagcagtggataagcaatttgctagatttttacttattggacatagagaaaagcttcgtcatatgatgacagaacaaaatattagtaagagacatgtagatataatacattcagaatcacctgcagattctgctagaattgcagtacaggcagtaaaatcaggaaatgcagatgtactaatgaaaggaaatgttccaactgctgtacttttaaaggcagttttaaataaagaatatggacttaggtcttctcatgtgctttctcatgtagcagcatttgaagtaagtggatttgagagattaatatatgtaactgatgctgcaatgaacatatcacctaagctagatgaattaaaacaaattcttgaaaatgccgtaggagtagcaagatctgtaggtgttcaaatgcctaaagtggcatgtcttgcagctgtagaaactgtaaatcctgctatggaagctactttaaatgccgcagcacttactcaaatgaatcatagaggtcaaattaaaaattgtgtagtagatgggccacttgctttggataatgccatatcacctttagcagcaagacacaaaaatataagcggtatcgttgcaggtgaagcagatatacttttggttcccagcattgaaactggaaatgttttatataaatcacttattcatttcgcaggagcaaaagtaggtgcaatcttagcaggtgcaaaagcacctatagcacttacttccagagcagattcagcagaaaataaattgtactccatagcacttgcactttgtacaagtgaagccaggcatgaagaagaataa

SEQ ID NO: 32 - DNA, 1068 bp, Clostridium acetobutylicum
  misc_feature (1)..(1068): ATCC824; Ca_Buk
atgtatagattactaataatcaatcctggctcgacctcaactaaaattggtatttatgacgatgaaaaagagatatttgagaagactttaagacattcagctgaagagatagaaaaatataacactatatttgatcaatttcaattcagaaagaatgtaattttagatgcgttaaaagaagcaaacatagaagtaagttctttaaatgctgtagttggaagaggcggactcttaaagccaatagtaagtggaacttatgcagtaaatcaaaaaatgcttgaagaccttaaagtaggagttcaaggtcagcatgcgtcaaatcttggtggaattattgcaaatgaaatagcaaaagaaataaatgttccagcatacatagttgatccagttgttgtggatgagcttgatgaagtttcaagaatatcaggaatggctgacattccaagaaaaagtatattccatgcattaaatcaaaaagcagttgctagaagatatgcaaaagaagttggaaaaaaatacgaagatcttaatttaatcgtagtccacatgggtggaggtacttcagtaggtactcataaagatggtagagtaatagaagttaataatacacttgatggagaaggtccattctcaccagaaagaagtggtggagttccaataggagatcttgtaagattgtgcttcagcaacaaatatacttatgaagaagtaatgaaaaagataaacggcaaaggcggagttgttagttacttaaatactatcgattttaaggctgtagttgataaagctcttgaaggagataagaaatgtgcacttatatatgaagctttcacattccaggtagcaaaagagataggaaaatgttcaaccgttttaaaaggaaatgtagatgcaataatcttaacaggcggaattgcgtacaacgagcatgtatgtaatgccatagaggatagagtaaaattcatagcacctgtagttagatatggtggagaagatgaacttcttgcacttgcagaaggtggacttagagttttaagaggagaagaaaaagctaaggaatacaaataa

SEQ ID NO: 33 - DNA, 1068 bp, Clostridium butyricum
  misc_feature (1)..(1068): Cu_Buk
atggcttacaaacttttaattataaatccaggatcaacatcaacaaaaataggagtatacgaagacgagaaggaattgtttgaagaaactttgagacacactaatgaagaattaaagcaatttgatgcaatatttgatcaattccagtttcgcaaagatgtaatcctaaaggtactttcagaaaaaaattttgatataaaaacattgtctgctgttgtaggaagaggcggcatgctcaaacctgtagaaggcggcacatatgcagtcaacgatgctatggtagaagatttaaaagtaggtgttcaaggtcctcatgcttccaatttaggcggcatacttgctaggtcaattgcagatgaaattggggtaccttcttttatagtagatcctgtcgttacagatgaactggcagatgtagcaagattgtcaggaactcctgacatacctagaaaatcaaagtttcatgcattaaatcaaaaggccgtagcaaaaagatatggtaaagaatcgggtaagggatatgaaaatcttaatcttgtagtagttcacatgggcggcggcgtttctgtaggtgctcacaatcatggaaaagttgttgacgttaataatgcacttgatggagatggacctttctctcctgaaagagcaggtagtgtacctgctggtgatcttattaaaatgtgttttagtggaaaatatagtgaaagtgaagtatatagtaaaatagttggaaaaggcggctttgtagggtacttaaatacaaatgatgtaaaaggaactatagataaaatggaagctggggataaggaatgtgaaaatatttataaggcattcctttatcagattacaaaggcaataggtgaaatgtcagctgcattaaatggaaaagttgatcagattgtacttacaggcggcattgcatacagtcctacattagttccagaccttaaagcaaatgtggaatggattgcacctgtaacagtttatcctggcgaggatgaattgcttgctcttgcacaaggtgctattcgtgtgctggatggagaagagaaagcaaaagtttattag

SEQ ID NO: 34 - DNA, 1107 bp, Bacillus licheniformis
  misc_feature (1)..(1107): Bli_Buk
atgcaagtgcaggaaaaaagaatattagttataaatccaggttctacatcaactaaaataggagtatttcatgacgatagatctatatttgaaaagtctataaggcatgatgaggcagaacttcagcagtatcaaactataatagatcaatacagcttcagaaagcaagctatacttgagaccttgcatgaacaaggtataaacataagtaagctagatgctgtatgtgcaagaggcggccttttgagaccaattgaaggcggcacgtatgaagtaaatgacgcgatgatagttgacttaaagaacggttatgctggacaacatgctagcaatcttggcggcataattgcccgtgagattgcagatggcttaaatattccagcttttatagtagatccagttgtagtagatgaaatggctcccatagcaaaaatttcaggcactccagcaattgaaagaaggtcaatttttcatgcattaaatcaaaaagctgtagctagaaaagctgcttggcaatttggaaaaagatatgaagatatgaaaatgatcattacacatatgggcggcggcataactatcggagttcactgcagaggaagagtcatagatgttaataatggattacatggagaaggacctctttcacctgaaagggcaggaactataccagcaggagatttaattgatatgtgcttttcaggtgaatatacaaaagatgaacttatgaagatgctagttggcggcggcggcttggcaggataccttggtactacagatgcagtaaaagtagaaaaaatgattaaagaaggtgatcaaaaagcagctttaatttatgaagctatggcctaccaaatagcaaaggaaattggtgcagcttcagcggttttaaaaggagaagttgatgttataatattaacaggcggccttgcttatggaaaatcttttatatcttcaattagacaatatatagattggattagtgacgtagtagtatttcctggagaaaatgaattacaggctttagctgaaggtgctttcagagtattaaatggagaagaagaagcaaaacagtatccaaatcaaagaagagaatcacatggaaattag

SEQ ID NO: 35 - DNA, 1311 bp, Clostridium autoethanogenum
  misc_feature (1)..(1311): CAETHG_1524
atgaacaacgacaactgcacgatcaaaattacccccgaagtttcgagagtcgatgaaccagtagatataaaaattaacggtttaccaaagaatgagaaggtaattatccgtgcggttagctctgactattactgtattaacgctagcatattggagataggcgataatacgttatgggagtcctacgcggtctttgagacagatgagtgtggaaacatcaattttgagaatgcagttccggtagatggcacgtacagtaactgtgacaaaatgggactgttttatagtatgcggcctaagcaaatccgtaagagcaaactgatccagaaactgtcctctatcaatgagaatcggaagtacaagattacattcacggtagagaaaaacggaaagatcataggaagcaaagaacacacgcgggtgtattgtgacgatacgatcaagagcatagatgtggtcgagaagaacctgctggctcgttatttcacttctaaagacaacatcaaacacccagcgataattgtgctttcgggatccgacggaagaattgaaaaagcccaagcaattgcagaattgttcgctatgcgcggctactcagcgctggctgtgtgttatttcggattagaagggacgcctgaagaccttaatatgatccccttggagtacgtcgaaaacgctgtcaagtggttgaaacgtcaggatacggtcgatgagaataaaatagccatctatggccggtctaaaggtggggagttggtacttttggcagcctctatgtttaaagacatcgcgtgcgtcattgccaatacgccatcctgctacgtttatgaaggaataaaaagtaacaagttgccttctcatcactcaagttggatgtacagaggtcgtgagattccttacttaaagttcaattttcacatcattttacgcttgataatcaaaatgatgaagaaggaaaaaggcgccttggcctggatgtataagaaactgatagaagaaggtgaccgggacaaggcgactatagcattagataagatcaacggatctgtcctgatgatatcgtctgctgcagatgagatatggccaagcaaaatgcacagtgaaaccgtttgttcaatattcgaaaaatctcacttcaagcatgagtataagcacatcacatttgctaagtcaggtcacatattgaccgttccgtttcaaagcatctatccgtcagagaaatatccttatgacgtggagtcctgggcaaaagccaatatggacagctggaatgagacgatcaaattcttagaaaaatgggcttccaagtaa

SEQ ID NO: 36 - DNA, 417 bp, Clostridium autoethanogenum
  misc_feature (1)..(417): CAETHG_0718
atgttgtacataaatgagacgaaggtagtcgtgcgctatgccgagacagataagatgggcattgtgcatcattcgaattattacatctattttgaagaggccagaactcaatttatcaaaaaaaccggaatttcgtactctcaaatggagaaggatggaatcatgtttccattagttgagagtaactgtcggtacctgcaaggtgccaaatacgaagatgaactgctgataaagacttggattaaagagttgacgcccgtcaaagccgagtttaattatagtgtaattcgggaaaacgaccagaaagaaatcgcaaaggggagtacattgcatgctttcgttaacaataatttcaagatcatcaacttgaagaagaatcacactgaattgtttaagaaacttcaaagcctgatttaa

SEQ ID NO: 37 - DNA, 387 bp, Clostridium autoethanogenum
  misc_feature (1)..(387): CAETHG_1780
atggactttagcaagctgtttaaggtaggctccacctatgtgagtgaatacatagtaaaacctgaggacactgcaaattttataggcaacaacggggtcgtgatgctttccaccccagctatgataaagtatatggaatacacaacgcttcatattgtagataatgtaattcccaaaaattatcgccctgtgggaactaaaattgatgtagagcacattaaaccgatccccgcaaatatgaaggtcgtagtcaaggtaacccttatttctatcgaagggaaaaagcttcgctacaacgtcgaggcgtttaacgaaaaaaactgcaaggtcgggtttggcatttacgaacaacaaatagtaaacttggaacaattccttaacagatag

SEQ ID NO: 38 - DNA, 861 bp, E. coli
  misc_feature (1)..(861): Eco_TesB
atgtcacaagctttaaaaaacttacttacattattaaatttagaaaaaatagaagaaggattattcagaggtcagtctgaagatcttggcttaaggcaggtatttggcggccaagtagtaggtcaagcattgtacgcagctaaggaaacagttccagaggaaagattagttcactcctttcattcctacttccttagaccaggagattcaaaaaaacctataatctatgatgtagaaacattaagggatggaaattccttttcagcaagaagagttgctgctatacaaaatggaaagcctatattttacatgactgcatctttccaagcgcccgaggccggatttgaacatcagaaaactatgccatcagcacctgctccagatgggttaccatcagaaactcaaattgcacagtcacttgcacatttacttcctccagtattaaaagataagtttatctgcgatagacctcttgaagtaagaccagttgaatttcataatccattaaaaggacatgtagctgaacctcataggcaggtatggattagggcaaacggatcagttccagatgatcttagagttcatcaatatcttcttggttatgctagtgatttgaatttcctaccagttgcacttcagcctcatggtattggattcttagagcctggaatccaaattgctactatagaccattccatgtggtttcatagaccgtttaatttaaatgaatggcttttatattcagttgaatctacttcagcttcttcagcaagaggatttgttagaggagagttctacacacaagatggagtattagtagcatctacagttcaagaaggagttatgagaaatcataattag

SEQ ID NO: 39 - DNA, 870 bp, Pseudomonas putida
  misc_feature (1)..(870): Ppu-TesB
atgtctcatgtattagatgatttagttgacttgttatcattggaatcaatagaagaaaatttatttagaggaagatcacaagatcttggatttagacagttgtatggcggccaggtacttggacaaagtttaagtgctgcaagtcaaacagtggaagatgcaagacatgttcactctcttcatggttacttcctaagacctggtgatgcttcactgccagtagtctattcagtagatagagttagagatggcggcagctttagtacaagacgtgtaactgctatacagaaaggtcagacaatttttacttgttcagccagcttccagtatgatgaagaaggatttgagcatcaggcacaaatgcctgatgttgtaggtcctgaaaatcttccaactgaagtagagttagcacatgctatggcagatcaattacctgaaagaattcgagataaggtgctttgtgctaagcctattgaaataagaccagttactgaaagagatccattcaatccaaaacccggtgatcctgtaaaatatgcttggttcagagctgacggaaatcttcctgatgtacctgctctccataaatatatgttagcttatgcaagtgattttggattacttacaacagcattactgcctcatggaaaatctgtatggcagagggatatgcagattgcttctctagatcattcattgtggttccatggaaatttaagagcagatcagtggcttttatatgctacagattctccttgggcaggcaactcaaggggcttttgtagaggttccatcttcaatcaggcaggacagcttgttgcttcatcgtctcaagaaggattaattagacatagaaaagattgggcataa

SEQ ID NO: 40 - DNA, 747 bp, Fibrobacter succinogenes
  misc_feature (1)..(747): S85; Fsu-TE2108
atggaaccagaagttactactaaaaactttgaagttagattctcagattgcgaccaccattccagacttaaattatctaatttatttttatttatggaagaaaccgctatagctgatgcagaacagaacggatttggaatttggaaaatgatgaaagcaggatatactacagttattacaagattaaaaataagacttcttcatcatccagtatggggagaaaagctttctatatctacttgggctaaggatattataaaagacaaagtttgtttaaaagattatagcattctagatgcacagggacactctatagcacaggcaacttcttcttggttattagtaaatatgaagactggaaaagcagaaaatccagcaaatgcaccatatcctataccactcatacaaggtaagaatgcattaccagaaatgatggatatattagacccacaggtagatcctcaaatagttgctacagaaattgcaaaatattcagaccttgacatgaataaacatgtaaaccactgtagatatgtagactgggttaccaattctttagatcctcaggagctcaaaagccgtagaattagatctatccagataaactacataagccagatccctttaggcggcaaagtaaatatagttagatttaagaatacgaatcatcatgcttatatatttggaactaatgcagatgatatgactcagtgtcattttcaagcaagaattggttttgctgattag

SEQ ID NO: 41 - DNA, 429 bp, Prevotella ruminicola
  misc_feature (1)..(429): Pru-TE655
atggctcaagaagtaaacaatggttatagacacatccttcctatacagataagattcaacgacgtagataaatttggacatgtaaataatacagtgtactttcaattttacgatactgccaaaactgaatactttgctactgtatgtgaagatgtagattgggaaagagttgctattgttgttgctaaaattgaggcagactttgttagccaaataaaagctggagatcatatagctgcaagaactagaacaatgaaaattggaaataagtctttccatttggagcaagatataatagatgttgatacattagaggtaaaatgtagatgtgcatccgtcatggttttatatgatttagagcatcaccagacaatgcctttccctgaagcatggagacaagctataagacaatatgatggattggaataa

SEQ ID NO: 42 - DNA, 414 bp, Prevotella ruminicola
  misc_feature (1)..(414): Pru-TE1687
atgaacgtagaaaaaatcgtagaaataataaacgcaaaaccaaatttatcaacagcattaggcatggaatttatatctaccccagaagtagatacatgcttggctaaaatgaaagtagatgaaagaaataggcagccttttggttttttatccggcggcgcttctttagcactggcagaaaatgtagcaggggtagcatcaagtgctttatgtcccggttgtgcatgtgttggtatagaagtaactggttcacatgttaaagctgtagtagagggagatactgtaactgcatttgcaaaaatgcttcacaagggtaaaactttacatgtatggaatgtagatataaaagatactgctggagatctcatatcaaatgtaagagttacaaattatgtaataaagcagaaaaaataa

SEQ ID NO: 43 - DNA, 882 bp, Cuphea palustris
  misc_feature (1)..(882): Cpa-TE
atgagaccaaatatgttaatggattcattcggattagaaagagttgtacaagatggattagtatttagacagtctttctccataagatcatatgaaatatgtgcagatagaacagcttccatagaaactgtaatgaatcatgttcaagaaacttctttaaatcagtgtaaatcaataggcttgttagatgatggttttgggcgaagcccggaaatgtgcaagagagaccttatatgggtggttactcgtatgaaaataatggtaaatagatatccaacctggggagatactattgaagtaagtacatggctcagccagtcaggaaaaataggtatgggtagagattggcttattagtgattgtaatacaggtgaaatattagttagagctacatcggtttatgcaatgatgaatcaaaaaactagaagattcagtaaattacctcatgaagttagacaggaatttgcacctcattttcttgactcaccaccagctatagaggataatgatggaaaacttcaaaagtttgacgtaaaaactggtgattctattagaaaaggacttacaccaggatggtatgatcttgatgtaaatcagcatgtatctaatgtaaaatacataggatggatattagaatctatgccaactgaagttcttgaaactcaagagttgtgtagtttaacattagaatatagaagagagtgcggtagagattctgttctagaatctgttacttcaatggatccttcaaaagttggagatagatttcaatatagacatttgttaaggttggaagatggagctgatattatgaaaggaagaactgaatggagacctaaaaatgcaggtactaatggtgcaataagcacaggtaaaacttaa

SEQ ID NO: 44 - DNA, 906 bp, Umbellularia californica
  misc_feature (1)..(906): Uca-TE
atgacattagaatggaaacctaaacctaaattgcctcagcttcttgatgaccattttggattacatggattagtatttagaagaacatttgcaatcagatcatatgaagttggtccggatcgatccacgtcaattctagcagtaatgaatcacatgcaagaagctacattgaaccatgcgaaaagcgttggaatacttggagatggttttggaacaactttagaaatgtcaaagagagacttaatgtgggtggttcgcagaacccatgtagctgtagaaagatatcctacttggggagatactgtagaagtagagtgttggataggagcttcaggaaataatggaatgaggagagatttccttgtacgtgattgcaaaactggagaaatattaacgcgctgtacgtcgttgagtgtacttatgaatacaagaactagaagattatccaccataccagatgaagtacgtggtgagataggtccagctttcattgataatgtagctgtaaaggatgatgaaattaaaaaacttcaaaaattaaatgatagtacggcagattatatacagggcggcctcacaccaagatggaacgatcttgatgtaaaccagcatgtaaacaacttaaagtatgtagcttgggtgtttgaaactgtacctgattcaatttttgaatctcatcacattagttcatttacattggaatatagaagagaatgtactagagattccgttttaagatctttaactactgtttcaggcggctcatcagaagcagggttggtatgtgatcatttgcttcaattagaaggcggcagtgaagtgcttagagctagaactgaatggagaccaaagttgactgattcttttaggggaatatcagttataccagcagagcctcgtgtataa

SEQ ID NO: 45 - DNA, 900 bp, Cuphea palustris
  misc_feature (1)..(900): CpFatB1.2-M4
atgtttgatagaaaaagtaaaagaccaagtatgttaatggattcatttgggttagaaagagtagttcaagacggattggtatttagacagagtttttccataagatcttatgaaatatgtgcagatagaactgcttcaatggaaacagttatgaatcatgtccaggaaacttctttaaatcagtgtaaatcaattggattacttgatgatggctttggaagaagtccagaaatgtgtaagagagatttaatctgggtggtaacaagaatgaagatcatggtaaatagatatccaacttggggagatactatagaggtatcaacttggttatcacaatcaggaaaaataggaatgggaagggattggttaatatctgattgtaatacaggggaaatacttgtgagagcaacatcagtatatgcaatgatgaatcaaaaaacaaggagattctctaaacttcctcacgaagtgagacaggaatttgcccctcatttccttgattctcctcctgctattgaagataatgatggaaaacttcaaaaatttgatgttaaaactggtgacagcattagaaagggacttacaccaggatggtacgaccttgatgtaaatcagcatgttagtaatgttaaatatattggctggatacttgaatcaatgccaactgaagttcttgaaacacaggaattatgttcccttacattagaatatagaagagaatgtggacgtgactcagtactggaatcagttacatcaatggatccatcaaaagttggtgacagattccagtatagacatcttcttagattagaggatggtgcggatataatgaagggaagaactgaatggcgtcctaaaaatgcaggaactaatggtgctatatctacaggtaaaacttaa

SEQ ID NO: 46 - DNA, 552 bp, Acinetobacter baylyi
  misc_feature (1)..(552): Aba-TEG17RA165R
atgggtaaaacaatattgattttaggagattcactttcagcaggctatagaataaatccagaacagggatgggtagcattacttcaaaagagattagatcagcaattccctaagcagcacaaggtaataaatgcttctgtatctggtgaaacaacttcaggtgcacttgccagactaccaaaattattaactacctacagaccaaatgtagttgttatagaattaggcggcaatgatgccttaagaggtcagccaccacagatgatccaaagcaaccttgaaaaacttatccagcacagccaaaaagcaaaatcaaaggtagtagtatttggtatgaagattccacctaactatggaactgcctacagccaggcttttgaaaacaattataaagtagtatctcagacatatcaggtaaaattacttcctttttttcttgatggagtagctggccacaagtctcttatgcaaaatgatcagattcaccctaatagaaaagcacaatccatacttttaaataatgcttatccttatataaagggagctttataa

SEQ ID NO: 47 - DNA, 399 bp, Escherichia coli
  misc_feature (1)..(399): Ecol_FadM
atgcaaactcagattaaagtaagaggatatcacttagatgtataccaacatgttaataatgcaagatatttagaattccttgaagaagcaagatgggatggattagaaaactcagatagcttccagtggatgacagcacataatattgcctttgtagttgtaaatataaacattaattatagaagaccagctgtactgagcgatttgcttacaataacttcacaattacagcagttaaatggaaaatcaggaattttaagtcaggtaataacacttgaaccagaaggacaagtagttgcagatgcgctgataacttttgtatgtatagatctaaaaactcaaaaagctcttgcacttgaaggtgaacttagagaaaaattagagcaaatggttaaataa

SEQ ID NO: 48 - DNA, 627 bp, Escherichia coli
  misc_feature (1)..(627): Ecol_TesA
atgatgaattttaacaatgtatttcgctggcatttaccatttttatttttggtcttacttacatttagagctgcagcagcagacacacttctcatccttggagattcactttcagcaggatatagaatgtctgcaagtgctgcatggccagcactccttaatgataaatggcaatcaaaaacttctgttgtaaatgctagtatttcaggagatacatctcaacaaggcttagctagattgcctgcacttttaaaacaacatcaaccaagatgggtattagtggaacttggcggcaatgatggtcttagagggtttcaaccacaacagacggaacagacactgaggcagatccttcaagatgttaaggctgccaatgctgaaccgcttttaatgcaaataaggcttccagcaaattatggaagaagatataatgaggcatttagtgctatatatcctaagttagcaaaagaatttgatgttcctcttcttccattttttatggaagaagtatatttaaagccacaatggatgcaggatgatggaatccacccaaatagagatgcacagccgtttatagcagattggatggctaaacagcttcaacctttagttaatcatgactcttaa

SEQ ID NO: 49 - DNA, 2577 bp, Clostridium acetobutylicum; identical to SEQ ID NO: 26
  misc_feature (1)..(2577): Cace_AdhE2

SEQ ID NO: 50 - DNA, 1482 bp, clostridium beijerinckii
  misc_feature (1)..(1482): Cbei_Ald
atgaatgttaaattagaaaatagatatagattatttattaatggagaatggagagatgcttcagatggaactacaataaagacttataatccagctaatggggaatttttagctgagattgcagatgctacagaaaaagatgtagatgatgctgttaattctgcaagacaagcctttgctacatggggcaaaactactgttgtagaaagagcaaatatattaaataaaattgctgatattattgaagaaaacgcagaatacttagctacagtagaaactttagataatggaaaaccaattagagagacaacaggagcagatattccattagcagcagatcactttagatattttgcaggtgttattcgtgctgatgagggttctgcaacaatgatagatgaaaatactttaaatttaatattaagtgagccaattggtgttgtagggcaaatagttccttggaattttccattcttaatggcggcttggaagttagctccagtgttagcagctggggatgtttcagtatttaagccatcaagtactacttcattaagcgtattagagcttatgaaactaatacaaaatattgttcctagtggtgtaattaatgttattacaggaaaaggatcaaagagcggtgaatatctgcaaaatcatgaaggacttgataaattagcatttacaggatctacagaagttggaagacaaattggacttgctgcggctaaacgtattattcctgcaactttagagcttggaggaaagtcagctaatattttctttagtgatgcaaatatgaacattgcattagaaggtattcaattgggaattttatttaaccaaggacaagtttgttctgcaggttctagaatatttgtacaagaagatttttatgatgaatttatggaaaaagcaattgctgcttttgaaaaagtcaaagttggaaacccattagatcctaatactcaaatgggagcgcaagttagtgaatctcaacttaaaaaaattcttaaatatatagaaataggtaaaaaagaaggcgcaaaagtagcgacaggaggggaaagattcattgagggtgatgctaagaatggatactttatgaaacctactttacttacagatgttactaatgatatgagaatagctagagaagaaatatttgggccagttggagttgtaattaaatttaagacagaagaagaagtcattgctatggcaaatgatagtgaatatggtttaggtggtggagtatttactactaacttaaatcgtgctataagagttgctaaagaaataaggactggacgtatatgggttaacacttataatactttcccagcaggtgcaacatttggtggatacaaagaatctggtataggcagggaaactcacaaagttatattagaagcgtatactcaaaagaaaaatatcatagttaatttatcagaaactcctggtggaatgtatataaagtaa

SEQ ID NO: 51 - DNA, 1986 bp, Marinobacter aquaeolei
  misc_feature (1)..(1986): Maqu2507
atgaactattttttaacaggcggcacaggatttataggaagatttttagtagaaaaattactagcaagaggcggcacagtttatgttcttgtacgtgagcaatctcaagataagttagaacgattaagagaaagatggggagctgatgataaacaagttaaagcagttataggagatcttactagtaagaatctaggcatagatgcaaaaacattaaagtcgttgaaaggtaatatagatcatgtatttcatcttgctgcagtttatgatatgggtgcagatgaagaagcacaggctgccactaatattgaaggtacaagagctgcagttcaagcagcagaggcaatgggagctaaacactttcatcatgtatcttcaatagctgcagcaggcttatttaagggaatatttagagaagacatgtttgaagaagcagaaaaactggatcatccttacttaaggacaaagcatgaatctgaaaaagtagttagagaagaatgtaaagttccatttagaatctaccgcccaggaatggtaattgggcactctgaaactggagagatggataaagtggacgggccatactatttctttaagatgatccagaaaataagacatgctcttccacaatgggtacctactataggaattgaaggcggcagattaaatattgtacctgtagattttgtagtagatgcactagatcatattgcacatcttgaaggagaagatggtaactgttttcacttagtagattcagatccatataaagtaggtgaaatattaaatattttttgtgaggctgggcatgcaccaagaatgggaatgcgtatagattcaaggatgtttggttttataccaccttttattagacaaagtataaagaatttacctcctgtaaaaagaattacgggagcactgcttgatgatatgggaatcccaccatccgtcatgagttttataaattatcctactagatttgatactagagaacttgaaagagtattaaaaggaacagatattgaagttccaagattgccttcttatgcacctgtaatttgggactactgggagagaaatctggatccagatctttttaaggatagaactttaaaggggacagtagagggtaaagtatgtgtagtaactggggcaacatcgggaataggtcttgcaactgctgaaaaattagctgaagctggagcaatattagtaattggagcaagaacaaaagaaacattagatgaagttgcagcttcactagaagcaaaaggcggcaatgtacatgcttatcagtgtgacttctctgatatggatgactgtgatagatttgtaaaaacagttttagataatcatggacatgtagatgttctagttaacaatgcaggaagaagtataagaagatctttagctctaagctttgacagattccatgattttgaaagaactatgcaattaaattacttcggttcagttagactaataatgggttttgctccagcaatgcttgaaagaagaagaggacatgttgtaaatatatcaagcataggtgtacttacaaatgcacctagattttctgcatatgtatcaagcaagagtgcccttgatgcattttcacgatgtgctgcagctgaatggtctgatagaaatgttacatttactactattaatatgcctttagttaagactcctatgattgcacctacaaagatatatgattcagtacctacattaacgccagatgaagctgctcagatggtagcagatgctatagtatataggcctaagagaattgctacaagattaggtgtatttgcacaggtactacatgcacttgcaccaaaaatgggcgaaattataatgaatacaggatatagaatgtttccagattctccagctgcagctggttctaagagtggtgaaaaacctaaagtgtccacagaacaggttgcatttgctgcaataatgagaggaatatactggtaa

SEQ ID NO: 52 - DNA, 888 bp, Acinetobacter baylyi
  misc_feature (1)..(888): Aca_acr1
atgaataaaaaattggaagcattatttagagaaaacgtaaagggcaaagtagcattaataacaggagcaagtagtggcataggattgactatagctaagagaattgctgctgctggtgcacatgtattgcttgtagctagaactcaagaaactcttgaagaggttaaagcagctatagaacaacaaggcggccaggcatccatatttccttgtgatttaactgacatgaatgctatagatcagctttctcaacaaataatggcaagtgtagatcatgtagactttcttataaacaatgctggaagaagtataagaagggcagtacatgaaagctttgatagatttcatgattttgaaagaactatgcagttaaactactttggtgcagttagactggtacttaacttattgccacatatgattaagagaaagaatggtcaaataataaatatttcgtctataggagtccttgctaatgcaaccagattttctgcttatgttgcttcaaaagctgcacttgatgctttttcgagatgtttgtctgctgaagttttaaagcataaaataagtataacatcaatctatatgcctttagttagaactccaatgattgccccaacaaaaatttacaaatatgtaccgactttgtcacctgaagaagcagcagatcttatagtatatgcaatagtaaaaagacctaaacgcatcgcaacacatcttggaagattagcttcaataacttatgccatagcacctgatataaataatatacttatgtcaattgggtttaatttatttccctcgtcaacagctgctcttggtgaacaagagaagttaaatttattacagagagcttatgctagattattccctggagagcattggtaa

SEQ ID NO: 53 - DNA, 984 bp, Clostridium saccharoperbutylacetonicum
  misc_feature (1)..(984): N1 4(HMT); DJ006_acr1
atgaaacatactattcaatcaccaataaattcaggatatggtttttctacaactgcaaaagaagttatagaaaatcttaatttacaaggtaagatagcaattgttactggaggttattcgggaatcggattggaaactgctaaagttttagccgaagctggcgccacagttatagtaccagcaagaaatattgagaaagcacagaaagccatagatggaattaaaaatatagagctaggaactttagatcttatggactctgattctataaatagttttgcagagaaatttatagcttccggaaggcccattaacatcttagtaaatagtgctggaatcatgactccacctttaatgagagataatagagggtatgaatctcaatttgctacaaatcatttaggacactttcaattaacagcaagactttggcctgctttaaaaaaagctggtagtgctagggttattgcagtttcatcaagagctcaacgtcttggaggtgttaattttgaagatcctaattttcaaaaaacagaatatgataaatggaaagcctatgcccaatcaaaatctgccaatatactttttgctgttgagcttgatagattaggtaaggaatatggagtaagagcttttgcagttcacccagggttaattccaactacagaccttggtagattttctcttgacggaaaagttactacacaagagcttaagaataaagataaaaaaactgctgataagcaacctgtaaatgaatttaaaaacattgagcaaggtgcagccacaccagtatggtgcgcaaccagccctttactaaatgagatgggtggtgtatattgtgaggattgcgatatctccgaggctgttacagctgatagcttaaaggaaaatggggtacgtccttgggccatagatacagatttagctaggagattgtggcaacttagcgaagaacttactggtattaagtttaatatttag

SEQ ID NO: 54 - DNA, 984 bp, Clostridium beijerinckii
  misc_feature (1)..(984): DJ008_acr1
atgaaaaatactactcaaacaccaataaattcaaaatataatttttttacaactgcaaaagatgttatagaagatatagacttgaaagataagattgcaattattactggcggatattctggaattgggatggaaacagcaaaagttttagctgaagctggtgcaacagtaataattcctgctagagatatagaaaaagcaaaagaagccatagctaaaattccaaatatagaaattgagcatttagaccttatggatccaatgtctattgatagtttcgcacaaaagtttataaattctcaaagatctcttcatattttaataaacagcgctggaataatggcacctccactaataagagacaaaagaggttatgaatctcaatttgcgacaaatcatctaggtcatttccagttaacagcacgactttggcctgctctaaaaaatgctaaaagtgctagagttatttcagtttcatcaagagcgcagcgtcttggcggagtcaattttgatgatccaaactttcaaaaaacagaatatgatagttggaaggcttatgctcaatcaaaatctgctaattcattattcgccgttgaattagacagattaggaaaaacccatggtgttagggctttctcagttcacccaggtttaattccaactacaaatcttggaagattctctgttaacggaaaagctactgtacaagaactaaaaactaatactagaaaagatgatactaatacaaaatcaaatgaatttaaaaccattgaacaaggtgcagcaacctctgtttggtgtgctacaaatagtattctagatggaatgggtggagtttactgtgaagactgcaatatagctgaagctgttccttatgacagtttgaaagataatggtgttcgtccttgggctatagataaaaagctagcaaaaaaactgtggatacttagtgaagatcttacaaatgttaaattcataatttaa

SEQ ID NO: 55 - DNA, 984 bp, Clostridium beijerinckii
  misc_feature (1)..(984): DJ052_acr1
atgaaaaatacaactcaagcaccaataaacagcaaatacaatttctttacaacagctaaggacgtaatagatggtataaatttaaaaggtaaaattgcaattgttacaggcggctattcaggtataggcatggaaactgcaaaggtattagctgaggctggagcaacagtaattatacctgcaagagatattgaaaaagctaaaggtgcaatggataatatcccaaatatagaaatcgaacatttggatttaatggatcctatgtccatagattcttttgcacaaaaatttatcaatagtcagagatcacttcatattcttataaactcagctggtattatggctccacctcttataagagataaaagaggatatgaatctcaatttgcaactaatcatcttggacattttcagcttactgctagactgtggccggccttgaaaaatgctaaatctgcaagagtaatatcagtgtcatctagagcccagagattaggcggcgtaaattttgatgatcctaactttcaaaaaacagaatacgattcatggaaggcttatgcacaaagcaaatcagctaatagtttgtttgcagtagaattagatagattaggaaaaacacatggtgttagagctttcagtgttcatcctggtcttattccaactacaaatctaggtagattttctgtaaatggtaagactacagtgcaggaattaaaaacaaacactagaaaagacgatacaaatacaaagtccaatgaattcaaaacaatagaacaaggagcagctacttcagtttggtgtgctacaaattctatacttgatggaatgggcggcgtatattgtgaagattgcaacattgcagaagcagttccatacgattcgttaaaagacaatggcgtaagaccatgggctattgataaaaatcttgccaagaaactttggatattaagtgaagagcttacaaatgtaaaatttataatttaa

SEQ ID NO: 56 - DNA, 984 bp, Clostridium beijerinckii
  misc_feature (1)..(984): DJ079_acr1
atgaaaaatactactcaaacaccaataaacagcaaatataattttagtactactgcaaaagatgtcatagatggtataaatcttaagggaaaaattgccattgtaacgggcggctatagcggtataggaatagagactgagaaagtacttgcagaagctggagcaactgttataattccagcccgtgatatcgagaaagctaaaggagcaatggataatattccaaatatagaaatagaacatttagatcttatggatccaatgtccattgacagttttgctcaaaaatttataaatagtcagcgtagtttgcatattcttataaattctgcagggattatggcacctccattgattcgtgataaaaggggctatgaaagtcaatttgcaacaaatcatttaggacattttcagcttacagctaggctatggccagctctaaaaaatgcaaagggtgcaagagtaatatctgtttcatcaagagcacagagacttggcggcgtaaattttgatgatccaaattttcaaaaaacggaatatgattcctggaaagcatatgctcaatcaaaaagtgctaattctctttttgctgtagaacttgatagactaggtaaaactcatggagtaagggcattttcagtacatccaggacttatacctactacaaatttaggtaggtttagtgtaaatggaaagacaactgtacaggaacttaaaacaaacactagaaaggatgatacaaatacaaaatcaaatgaattcaaaactatagaacagggtgctgcaacttctgtatggtgtgcaactaacagcatacttgatggtatgggcggcgtttattgtgaagattgtaatattgcagaagcagttccatatgactcactgaaagataacggagttaggccttgggctattgacaaagatttagcaaaaaagctgtggatacttagtgaagatcttacaaatgtaaagttcataatttaa

SEQ ID NO: 57 - DNA, 984 bp, Clostridium beijerinckii
  misc_feature (1)..(984): DJ322_acr1
atgaaaaacactactcaaacaccaataaattcaaaatataatttttctacaactgcaaaagatgttatagatggtataaacttgaaaggtaaaattgcaattgttactggcggatattctggaattgggatagaaacagcaaaagttttagctgaagctggtgcaacagtaataattcctgctagagatatagaaaaagcaaaaggagctatggataatattccaaatatagaaattgaacatttagaccttatggatccaatgtctattgatagtttcgcacaaaagtttataaattctcaaagatctcttcatattttaataaatagcgctggaataatggcacctccactaataagggacaaaagaggttatgaatctcaatttgcgacaaatcatctaggtcatttccagttaacagcacgactttggcctgctctaaaaaatgctaaaggtgctcgagttatttcagtttcatcaagagcgcagcgtcttggcggagtcaattttgatgatccaaactttcaaaaaacagaatatgatagttggaaggcttatgctcaatcaaaatccgctaattcattattcgccgttgaattagacagattaggaaaaacccatggtgttagggctttctcagttcacccaggtttaattccaactacaaatcttggaagattctctgttaacggaagagctactgtacaagaactaaaaactaacactagaaaagatgatactaacacaaaatcaaatgaatttaaaaccattgaacagggtgcagcaacctctgtttggtgtgctacaaatagtattctagatggaatgggtggagtttactgtgaagactgcaatatagctgaagctgttccttatgacagtttgaaagataatggtgttcgtccttgggctatagataaagatctagcaaaaaaactgtggatacttagtgaagatcttacaaatgttaaattcataatttaa

(19) United States
(12) Patent Application Publication
(10) Pub. No.: US 2022/0282289 A1
(43) Pub. Date: Sep. 8, 2022

(54) RECOMBINANT MICROORGANISMS AND USES THEREFOR

(71) Applicant: LanzaTech, Inc., Skokie, IL (US)

(72) Inventors: Shivani Garg, Park Ridge, IL (US); Michael Koepke, Chicago, IL (US)

(21) Appl. No.: 17/653,913

(22) Filed: Mar. 8, 2022

Related U.S. Application Data
(60) Provisional application No. 63/158,336, filed on Mar. 8, 2021.

(51) Int. Cl. C12P 7/6409; C12N 15/52; C12N 15/74

(52) U.S. Cl. CPC C12P 7/6409; C12N 15/52; C12N 15/74; C12Y 101/0133; C12Y 103/01009; C12Y 203/01009; C12Y 402/01017; C12N 2800/101

(57) ABSTRACT

Microorganisms are genetically engineered to produce various chemicals for industrial use. The microorganisms are carboxydotrophic acetogens. The microorganisms produce acetyl-CoA using the Wood-Ljungdahl Pathway for fixing CO/CO₂. A reverse beta-oxidation pathway cycle from a microorganism that contains such a group of enzymes is introduced. Additionally, primers and extenders, and/or genes encoding for enzymes that generate primers and extenders may also be introduced. Product synthesis can be effected by improved promoters or enzyme designs that are catalytically more efficient. Similarly, product synthesis may also be improved by deleting competing reactions.

### CROSS REFERENCE TO RELATED APPLICATIONS

This application claims the benefit of U.S. Provisional Patent Application No. 63/158,336, filed Mar. 8, 2021, the entirety of which is incorporated herein by reference.

### GOVERNMENT RIGHTS

This disclosure was made with government support under Cooperative Agreement DE-EE0008354 awarded by the Department of Energy.

### REFERENCE TO A SEQUENCE LISTING

The application contains a Sequence Listing which has been submitted electronically in ASCII format and is hereby incorporated by reference in its entirety. Said ASCII copy, created on Feb. 14, 2022, is named LT204US1-Sequences.txt and is 91,877 bytes in size.

### FIELD OF THE DISCLOSURE

This disclosure relates to recombinant microorganisms and methods for the production of drop-in fuels, fuel additives, and chemical building blocks that arise from an engineered reversal of the β-oxidation cycle by microbial fermentation of a substrate comprising CO, CO₂, and/or H₂.

### BACKGROUND

With rising concerns over climate change and contribution of petrochemicals to the same, new biological routes are emerging for production of industrial chemicals and fuels. Reversal of beta-oxidation pathway (rBOX) is one such biological route that can enable access to hundreds of diverse chemical molecules via an iterative production platform.

The rBOX pathway so far has been demonstrated successfully in hosts such as Escherichia coli and yeast to convert primarily sugars or 3-carbon substrates (such as glycerol) to multiple classes of products. Its functionality has not been demonstrated in organisms, such as Clostridium autoethanogenum, that ferment gases utilizing the ancient Wood-Ljungdahl pathway.

This disclosure provides recombinant microorganisms and uses thereof to produce a diverse class of products, including alcohols, acids, diols, diacids, ketoacids, hydroxyacids, fatty acid methyl esters, by expressing the rBOX pathway in a CO/CO₂ utilizing host.

### SUMMARY

The disclosure generally provides, inter alia, a genetically engineered microorganism and methods for the production of primary alcohols, 1,4-diols, 1,6-diols, diacids, trans Δ² fatty alcohols, β-keto alcohols, 1,3-diols, β-hydroxy acids, carboxylic acids, or hydrocarbons by microbial fermentation of a substrate comprising CO, CO₂, and/or H₂, and recombinant microorganisms of use in such methods.

In a first aspect, the disclosure provides an genetically recombinant microorganism capable of producing from primary alcohols, 1,4-diols, 1,6-diols, diacids, trans Δ² fatty alcohols, β-keto alcohols, 1,3-diols, β-hydroxy acids, carboxylic acids, or hydrocarbons and optionally one or more other products by fermentation of a substrate comprising CO, CO₂, and/or H₂.

In one particular embodiment, the microorganism is adapted to express one or more enzymes (or one or more subunits thereof) in the reverse β-oxidation pathway, e.g., in a reverse biosynthetic direction, which enzymes are not naturally present in a parental microorganism from which the recombinant microorganism is derived. In another embodiment, the microorganism is adapted to over-express one or more enzymes (or one or more subunits thereof) in the reverse β-oxidation pathway, which enzymes are naturally present in a parental microorganism from which the recombinant microorganism is derived. In one embodiment, the microorganism is adapted to express one or more enzymes (or one or more subunits thereof) in the reverse β-oxidation pathway which are not naturally present in a parental microorganism and over-express one or more enzymes (or one or more subunits thereof) in the reverse β-oxidation pathway which are naturally present in a parental microorganism. The reverse β-oxidation pathway is also cyclic and iterative. In another embodiment, the β-oxidation pathway is driven in reverse for as many cycles, as desired. In one embodiment, the β-oxidation pathway is expressed in the absence of any naturally occurring substrates. In one embodiment, the reverse β-oxidation pathway is functionally expressed. In one embodiment, the CoA thioester intermediates can be converted to useful products by the action of different types of termination enzymes.

This pathway operates with coenzyme-A (CoA) thioester intermediates and directly uses, for example, acetyl-CoA for acyl-chain elongation, and characteristics that enable product synthesis used in combination with endogenous dehydrogenases and thioesterases to synthesize (Cₙ)-alcohols, fatty acids, β-hydroxy-, β-keto-, and trans-Δ²-carboxylic acids.

This pathway can be further extended using the same enzymes or engineered variants thereof that have specificity for higher chain length to produce, including but not limited to, a range of C4, C6, C8, C10, C12, C14 alcohols, ketones, enols or diols. Different type of molecules can be obtained also by using primer or extender units different than acetyl-CoA in a thiolase step.

In one embodiment, a genetically engineered microorganism capable of producing a product from a gaseous substrate, wherein the microorganism comprises an iterative pathway comprising:
a) a nucleic acid encoding a group of enzymes that are capable of catalyzing the conversion of (Cₙ)-acyl CoA to β-ketoacyl-CoA;
b) a nucleic acid encoding a group of exogenous enzymes capable of catalyzing the conversion of β-ketoacyl-CoA to β-hydroxyacyl-CoA;
c) a nucleic acid encoding a group of exogenous enzymes capable of catalyzing the conversion of β-hydroxyacyl-CoA to trans-Δ²-Enoyl-CoA;
d) a nucleic acid encoding a group of exogenous enzymes capable of catalyzing the conversion of trans-Δ²-Enoyl-CoA to (Cₙ₊₂) acyl-CoA;
e) one or more termination enzymes; and wherein the microorganism is a C1-fixing bacteria comprising a disruptive mutation in a thioesterase.

In one embodiment, the nucleic acid encoding a group of enzymes that are capable of catalyzing the conversion of (Cₙ)-acyl CoA to β-ketoacyl-CoA is a thiolase, an acyl-CoA acetyltransferase, or a polyketide synthase; the nucleic acid encoding a group of enzymes that are capable of catalyzing the conversion of β-ketoacyl-CoA to β-hydroxyacyl-CoA is a 13-Ketoacyl-CoA reductase or a β-hydroxyacyl-CoA dehydrogenase; the nucleic acid encoding a group of exogenous enzymes capable of catalyzing the conversion of β-ketoacyl-CoA to trans-Δ²-Enoyl-CoA is a β-hydroxyacyl-CoA dehydratase; the nucleic acid encoding a group of exogenous enzymes capable of catalyzing the conversion of trans-Δ²-Enoyl-CoA to (C_{b+2}) acyl-CoA is a trans-Enoyl-CoA reductase or butyryl-CoA dehydrogenase/electron transferring flavoprotein AB (Bcd-EtfAB).

In one embodiment, a nucleic acid encoding a group of termination enzymes is selected from alcohol-forming coenzyme-A thioester reductase, an aldehyde-forming CoA thioester reductase, an alcohol dehydrogenase, a thioesterase, an acyl-CoA:acetyl-CoA transferase, a phosphotransacylase and a carboxylate kinase; aldehyde ferredoxin oxidoreductase; an aldehyde-forming CoA thioester reductase, an aldehyde decarbonylase, alcohol dehydrogenase; aldehyde dehydrogenase, an acyl-CoA reductase, or any combination thereof. In one embodiment, a termination enzyme is phosphate butyryltransferase (Ptb) and exogenous butyrate kinase (Buk) (Ptb-Buk). In one embodiment, a termination enzyme is thioesterase. In one embodiment, one or more termination enzymes are selected.

In one embodiment, the microorganism comprises one or more exogenous nucleic acids adapted to increase expression of one or more nucleic acids native to the parental microorganism and which one or more nucleic acids encode one or more of the enzymes (or one or more subunits thereof) referred to herein before.

In one embodiment, the one or more exogenous nucleic acid adapted to increase expression is a regulatory element. In one embodiment, the regulatory element is a promoter.

In one embodiment, the promoter is a constitutive promoter. In one embodiment, the promoter is selected from the group comprising Wood-Ljungdahl gene cluster or Phosphotransacetylase/Acetate kinase operon promoters.

In one embodiment, the microorganism comprises one or more exogenous nucleic acids encoding and adapted to express one or more of the enzymes (or one or more subunits thereof) referred to herein before. In one embodiment, the microorganisms comprise one or more exogenous nucleic acid encoding and adapted to express at least two of the enzymes (or one or more subunits thereof).

In one embodiment, the one or more exogenous nucleic acid is a nucleic acid construct or vector, in one particular embodiment a plasmid, encoding one or more of the enzymes referred to hereinbefore in any combination.

In one embodiment, two or more enzymes on a plasmid are arranged in a single operon in any order, or in multiple operons in any order.

In one embodiment, the exogenous nucleic acid is an expression plasmid.

In one embodiment, the parental microorganism is selected from the group of anaerobic acetogens.

In one particular embodiment, the parental microorganism is selected from the group of carboxydotrophic acetogenic bacteria, in one embodiment from the group comprising Clostridium autoethanogenum, Clostridium ljungdahlii, Clostridium ragsdalei, Clostridium carboxidivorans, Clostridium drakei, Clostridium scatologenes, Clostridium aceticum, Clostridium formicoaceticum, Clostridium magnum, Butyribacterium methylotrophicum, Acetobacterium woodii, Alkalibaculum bacchii, Blautia producta, Eubacterium limosum, Moorella thermoacetica, Moorella thermautotrophica, Sporomusa ovata, Sporomusa silvacetica, Sporomusa sphaeroides, Oxobacter pfennigii, and Thermoanaerobacter kivui.

In one embodiment the parental microorganism is Clostridium autoethanogenum or Clostridium ljungdahlii. In one particular embodiment, the microorganism is Clostridium autoethanogenum DSM23693. In another particular embodiment, the microorganism is Clostridium ljungdahlii DSM13528 (or ATCC55383).

In a second aspect, the disclosure provides a nucleic acid encoding one or more enzymes (or one or more subunits thereof) which when expressed in a microorganism allows the microorganism to produce Cₙ₊₂ Acetoacid, Cₙ₊₂ 3-OH-acid, Cₙ₊₂ Enoate, Cₙ₊₂ 1-acid, Cₙ₊₂ ketone, Cₙ₊₂ methyl-2-ol, Cₙ₊₂ 1,3-diol, 1,4-diol, 1,6-diol, Cₙ₊₂ 2-en-1-ol, Cₙ₊₂ 1-alcohol, diacids, or any combination thereof, by fermentation of substrate comprising CO and/or CO₂. For example, a Cₙ₊₂ ketone may be acetone.

In one embodiment, the nucleic acid encodes two or more enzymes (or one or more subunits thereof) which when expressed in a microorganism allows the microorganism to produce primary alcohols, 1,4-diols, 1,6-diols, diacids, trans Δ² fatty alcohols, β-keto alcohols, 1,3-diols, β-hydroxy acids, carboxylic acids, or hydrocarbons, by fermentation of substrate comprising CO.

In one embodiment, the enzymes are chosen from thiolase, acyl-CoA acetyltransferase, or a polyketide synthase, and/or a functionally equivalent variant of any one or more thereof.

In one embodiment, the nucleic acid comprises nucleic acid sequences encoding β-Ketoacyl-CoA reductase and/or a β-hydroxyacyl-CoA dehydrogenase, or a functionally equivalent variant of any one or more thereof, in any order.

In one embodiment, the nucleic acid encoding thiolase has the sequence of SEQ ID NO: 1 to SEQ ID NO: 6, or is a functionally equivalent variant thereof. In one embodiment, the nucleic acid encoding β-Ketoacyl-CoA reductase has the sequence of SEQ ID NO: 7 to SEQ ID NO: 14, or is a functionally equivalent variant thereof. In one embodiment, the nucleic acid encoding β-hydroxyacyl-CoA dehydrogenase has the sequence of SEQ ID NO: 15 to SEQ ID NO: 22, or is a functionally equivalent variant thereof. In one embodiment, the nucleic acid encoding trans-Enoyl-CoA reductase has the sequence of SEQ ID NO: 23 to SEQ ID NO: 28, or is a functionally equivalent variant thereof.

In one embodiment, the nucleic acids of the disclosure further comprise a promoter. In one embodiment, the promoter allows for constitutive expression of the genes under its control. In a particular embodiment a Wood-Ljungdahl cluster promoter is used. In another particular embodiment, a Phosphotransacetylase/Acetate kinase operon promoter is used. In one particular embodiment, the promoter is from C. autoethanogenum.

In a third aspect, the disclosure provides a nucleic acid construct or vector comprising one or more nucleic acid of the second aspect.

In one particular embodiment, the nucleic acid construct or vector is an expression construct or vector. In one particular embodiment, the expression construct or vector is a plasmid.

In a fourth aspect, the disclosure provides host organisms comprising any one or more of the nucleic acids of the seventh aspect or vectors or constructs of the third aspect.

In a fifth aspect, the disclosure provides a composition comprising an expression construct or vector as referred to in the third aspect of the disclosure and a methylation construct or vector.

Preferably, the composition is able to produce a recombinant microorganism according to the first aspect of the disclosure.

In one particular embodiment, the expression construct/vector and/or the methylation construct/vector is a plasmid.

In a sixth aspect, the disclosure provides a method of producing a product, the method comprising culturing the engineered microorganism of claim 1, in the presence of a gaseous substrate.

In one embodiment the method comprises wherein the gaseous substrate comprises a C1-carbon source comprising CO, CO₂, and/or H₂.

In one embodiment the method comprises wherein the product is selected from (Cₙ)-alcohols, primary alcohols, trans Δ² fatty alcohols, β-keto alcohols, 1,3-diols, 1,4-diols, 1,6-diols, diacids, β-hydroxy acids, β-ketoacids carboxylic acids, fatty acids, fatty acid methyl esters, ketoacids, hydrocarbons, or any combination thereof.

In particular embodiments of the method aspects, the microorganism is maintained in an aqueous culture medium.

In particular embodiments of the method aspects, the fermentation of the substrate takes place in a bioreactor.

Preferably, the substrate comprising CO and/or CO₂ is a gaseous substrate comprising CO and/or CO₂. In one embodiment, the substrate comprises an industrial waste gas. In certain embodiments, the gas is steel mill waste gas or syngas.

In a particular embodiment, the substrate is a substrate comprising CO.

In embodiments of the disclosure where the substrate comprises CO₂, but no CO, the substrate preferably also comprises H₂.

In one embodiment, the substrate comprises CO and CO₂. In one embodiment, the substrate comprises CO₂ and H₂. In another embodiment, the substrate comprises CO, CO₂, and H₂.

In one embodiment, the substrate will typically contain a major proportion of CO, such as at least about 20% to about 100% CO by volume, from 20% to 70% CO by volume, from 30% to 60% CO by volume, and from 40% to 55% CO by volume. In particular embodiments, the substrate comprises about 25%, or about 30%, or about 35%, or about 40%, or about 45%, or about 50% CO, or about 55% CO, or about 60% CO by volume.

In certain embodiments the methods further comprise the step of recovering a product selected from primary alcohols, 1,4-diols, 1,6-diols, diacids, trans Δ² fatty alcohols, β-keto alcohols, 1,3-diols, β-hydroxy acids, carboxylic acids, or hydrocarbons, and optionally one or more other products from the fermentation broth.

In a seventh aspect, the disclosure provides any primary alcohol when produced by the method of the sixth aspect.

In another aspect, the disclosure provides a method for the production of a microorganism of the first aspect of the disclosure comprising transforming a parental microorganism with one or more exogenous nucleic acid such that the microorganism is capable of producing Cₙ₊₂ Acetoacid, Cₙ₊₂ 3-OH-acid, Cₙ₊₂ Enoate, Cₙ₊₂ 1-acid, Cₙ₊₂ ketone, Cₙ₊₂ methyl-2-ol, Cₙ₊₂ 1,3-diol, 1,4-diol, 1,6-diol, Cₙ₊₂ 2-en-1-ol, Cₙ₊₂ 1-alcohol, diacids, or any combination thereof, and optionally one or more other products, by fermentation of a substrate comprising CO and/or CO₂, wherein the parental microorganism is not capable of producing Cₙ₊₂ Acetoacid, Cₙ₊₂ 3-OH-acid, Cₙ₊₂ Enoate, Cₙ₊₂ 1-acid, Cₙ₊₂ ketone, Cₙ₊₂ methyl-2-ol, Cₙ₊₂ 1,3-diol, 1,4-diol, 1,6-diol, Cₙ₊₂ 2-en-1-ol, Cₙ₊₂ 1-alcohol, diacids, or any combination thereof by fermentation of a substrate comprising CO and/or CO₂.

In one particular embodiment, a parental microorganism is transformed with one or more exogenous nucleic acid adapted to express one or more enzymes in the reverse β-oxidation pathway which are not naturally present in the parental microorganism. In another embodiment, a parental microorganism is transformed with one or more nucleic acid adapted to over-express one or more enzymes in the reverse β-oxidation pathway which are naturally present in the parental microorganism. In another embodiment, a parental microorganism is transformed with one or more exogenous nucleic acid adapted to express one or more enzymes in the reverse β-oxidation pathway which are not naturally present in the parental microorganism and over-express one or more enzymes in the reverse β-oxidation pathway which are naturally present in the parental microorganism.

In certain embodiments, the one or more enzymes are as herein before described.

In certain embodiment, the parental microorganism is as herein before described.

According to one embodiment a process is provided for converting CO or CO₂ into primary alcohol. A gaseous CO-containing and/or CO₂-containing substrate is passed to a bioreactor containing a culture of carboxydotrophic, acetogenic bacteria in a culture medium such that the bacteria convert the CO and/or CO₂ to a primary alcohol. The carboxydotrophic acetogenic bacteria are genetically engineered to express an enzyme in the reverse β-oxidation pathway. They also express an enzyme in the reverse β-oxidation pathway, whether native or exogenous. The primary alcohol is recovered from the bioreactor.

According to another embodiment an isolated, genetically engineered, carboxydotrophic, acetogenic bacterium is provided that comprises a nucleic acid encoding a group of enzymes in the reverse β-oxidation pathway. The nucleic acid is exogenous to the host bacteria. The bacteria express the group of enzymes in the reverse β-oxidation pathway and the bacteria acquire the ability to generate primary alcohols, trans Δ² fatty alcohols, β-keto alcohols, 1,3-diols, 1,4-diols, 1,6-diols, diacids, β-hydroxy acids, carboxylic acids, or hydrocarbons. The group of enzymes in the reverse β-oxidation pathway are typically at least 85% identical to the amino acid sequence encoded by any one of the nucleotide sequences of SEQ ID NO: 1 to SEQ ID NO: 57. In one embodiment, termination enzymes such as thioesterases, acyl-CoA reductases, or phosphotransacylase, carboxylate kinase, are selected because these pull the acyl-CoA intermediates from the rBOX pathway and drive the metabolic flux through the pathway.

The bacteria may further comprise an exogenous nucleic acid encoding acetyl-Coenzyme A carboxylase. The nucleic acid may be operably linked to a promoter. The nucleic acid may have been codon optimized. The nucleic acid or the encoded carboxylase may be from a nonsulfur, photosynthetic bacterium. The bacteria may be selected from the group consisting of Clostridium autoethanogenum, Clostridium ljungdahlii, Clostridium ragsdalei, Clostridium carboxidivorans, Clostridium drakei, Clostridium scatologenes, Clostridium aceticum, Clostridium formicoaceticum, Clostridium magnum, Butyribacterium methylotrophicum, Acetobacterium woodii, Alkalibaculum bacchii, Blautia producta, Eubacterium limosum, Moorella thermoacetica, Moorella thermautotrophica, Sporomusa ovata, Sporomusa silvacetica, Sporomusa sphaeroides, Oxobacter pfennigii, and Thermoanaerobacter kivui. The donor bacterium of the exogenous nucleic acid may be a nonsulfur, photosynthetic bacterium such as, Chloroflexus aurantiacus, Metallosphaera, and Sulfolobus spp.

The genetically engineered bacteria may be cultured by growing in a medium comprising a gaseous carbon source. The carbon source may comprise CO and/or CO₂, which may be used as either or both of an energy source or a carbon source. The bacteria may optionally be grown under strictly anaerobic conditions. The carbon source may comprise an industrial waste product or off-gas.

The disclosure may also be said broadly to consist in the parts, elements and features referred to or indicated in the specification of the application, individually or collectively, in any or all combinations of two or more of said parts, elements or features, and where specific integers are mentioned herein which have known equivalents in the art to which the disclosure relates, such known equivalents are deemed to be incorporated herein as if individually set forth.

### BRIEF DESCRIPTION OF THE DRAWINGS

FIG. 1: Engineered reversal of β-oxidation pathway for production of alcohols and acids in a C1 gas-fermenting organism. Genes encoding thiolase, β-ketoacyl-CoA reductase, β-hydroxyacyl-CoA dehydratase, and enoyl-CoA reductase constitute the core pathway genes. Termination enzymes, including thioesterase, phosphotransacylase, carboxylate kinase, acyl-CoA reductase, aldehyde reductase, Ferredoxin-dependent aldehyde oxidoreductase, enable conversion of acyl-CoAs generated through the rBOX pathway into corresponding alcohols or acids.

FIGS. 2A-2B: Demonstration of rBOX pathway in C. autoethanogenum (production of butanol, hexanol, and octanol) by heterologously expressing enzymes with thiolase, β-ketoacyl-CoA reductase, β-hydroxyacyl-CoA dehydratase and enoyl-CoA reductase/butyryl-CoA dehydrogenase-electron transferring protein AB functionalities. Experiments were performed in 250 ml Schott bottles with 10 ml minimal medium, gassed with synthetic syngas (n=3). Error bars represent standard deviation.

FIGS. 3A-3B: Heterologous expression of termination enzymes (including thioesterase, phosphotransacylase/carboxylate kinase, or acyl-CoA reductase) in addition to the core rBOX pathway enzymes to improve medium chain (C4-C8) alcohol production. S01: control strain. S11-16: strains with termination enzyme expressed. Experiments were performed in 250 ml Schott bottles with 10 ml minimal medium, gassed with synthetic syngas (n=3). Error bars represent standard deviation.

FIGS. 4A-4B: Improvement in hexanol selectivity by varying the gene variants for core rBOX enzymes and termination enzymes. Experiments were performed in 250 ml Schott bottles with 10 ml minimal medium, gassed with synthetic syngas (n=3). Error bars represent standard deviation.

FIGS. 5A-5B: Growth and metabolite profile of S25 compared to chassis strain (control without rBOX pathway genes). Experiments were performed in 250 ml Schott bottles with 10 ml minimal medium, gassed with synthetic syngas (n=3). Bottles were re-gassed every time after sampling. Error bars represent standard deviation.

FIGS. 6A-6C: Characterization of strain S25 in 1.5 L CSTR (batch mode) with a synthetic gas blend (50% CO, 10% H₂, 30% CO₂, and 10% N2).

FIGS. 7A-7B. Acid to alcohol conversion rate determined for strain S32 in 1.5 L CSTR (batch mode) run with a synthetic gas blend (50% CO, 10% H₂, 30% CO₂, and 10% N2).

FIGS. 8A-8B. Improvement in selectivity to hexanol via rearrangement of gene order on plasmids. Build 14 strains, i.e., B14.S1, B14.S2, and B14.S3 have same genes as S26, S28, and S29 respectively, but arranged in a different order on two plasmids. These strains were tested in in 250 ml Schott bottles with 10 ml minimal medium, gassed with synthetic syngas (n=3). Bottles were re-gassed every time after sampling. Error bars represent standard deviation.

### DETAILED DESCRIPTION

The following description of embodiments is given in general terms. The disclosure is further elucidated from the disclosure given under the heading “Examples” herein below, which provides experimental data supporting the disclosure, specific examples of various aspects of the disclosure, and means of performing the disclosure.

The inventors have surprisingly been able to engineer a carboxydotrophic acetogenic microorganism to produce alcohols by fermentation of a substrate comprising CO and/or CO₂. This offers an alternative means for the production of primary alcohols which may have benefits over the current methods for the production of primary alcohols. In addition, it offers a means of using carbon monoxide from industrial processes which would otherwise be released into the atmosphere and pollute the environment. In this disclosure, a genetically engineered microorganism expresses enzymes from the reverse β-oxidation pathway, which resulted in production of primary alcohols from a gaseous substrate.

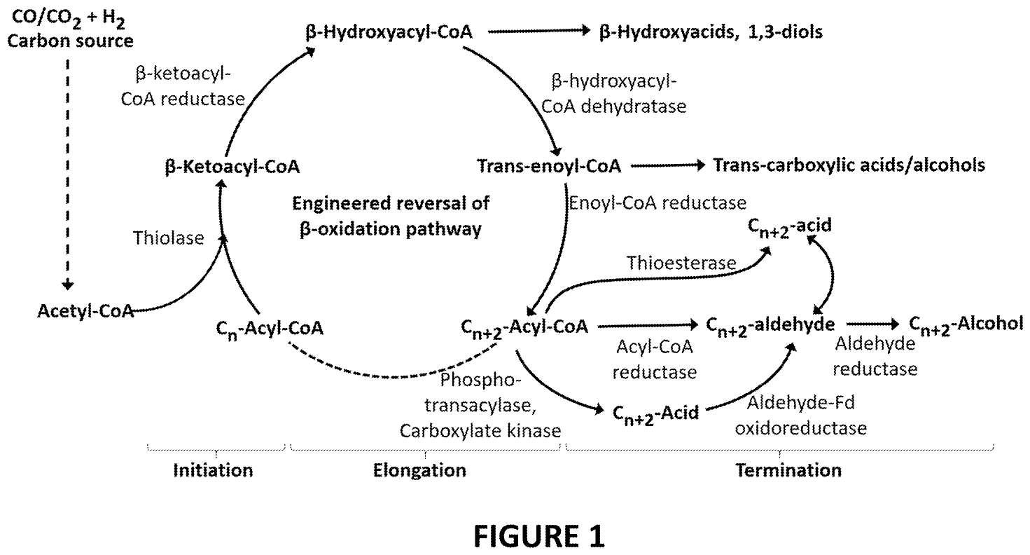

In engineering the microorganisms of the disclosure, the inventors have surprisingly been able to genetically engineer a microorganism capable of producing a product from a gaseous substrate, wherein the microorganism comprises an iterative pathway comprising catalyzing the conversion of (Cₙ)-acyl CoA to β-ketoacyl-CoA; catalyzing the conversion of β-ketoacyl-CoA to β-hydroxyacyl-CoA; catalyzing the conversion of β-hydroxyacyl-CoA to trans-Δ²-Enoyl-CoA; and catalyzing the conversion of trans-Δ²-Enoyl-CoA to (Cₙ₊₂) acyl-CoA; and one or more termination enzymes; and wherein the microorganism is a C1-fixing bacteria comprising a disruptive mutation in a thioesterase, as illustrated in FIG. 1. This pathway can be further extended using the same enzymes or engineered variants thereof that have specificity for higher chain length to produce, including but not limited to, a range of C4, C6, C8, C10, C12, C14 alcohols, ketones, enols or diols. Different type of molecules can be obtained also by using primer or extender units different than acetyl-CoA in a thiolase step. This provides for sustainable fermentation to produce primary alcohols using a substrate comprising CO and/or a substrate comprising CO₂.

Primers and extenders are selected from oxalyl-CoA, acetyl-CoA, malonyl CoA, succinyl-CoA, hydoxyacetyl-CoA, 3-hydroxyproprionyl-CoA, 4-hydroxybutyryl-CoA, 2-aminoacetyl-CoA, 3-aminopropionyl-CoA, 4-aminobutyryl-CoA, isobutyryl-CoA, 3-methyl-butyryl-CoA, 2-hydroxyproprionyl-CoA, 3-hydroxybutyryl-CoA, 2-aminoproprionyl-CoA, propionyl-CoA, and valeryl-CoA. Moreover, the bacteria express the group of enzymes in the reverse β-oxidation pathway and the bacteria acquire the ability to generate primary alcohols, trans Δ² fatty alcohols, β-keto alcohols, 1,3-diols, 1,4-diols, 1,6-diols, diacids, β-hydroxy acids, carboxylic acids, or hydrocarbons. In one embodiment, acetyl-CoA is the primer/starter molecule, which leads to synthesis of even-chained n-alcohols and/or carboxylic acids. In another embodiment, propionyl-CoA is the starter/primer molecule, which enables the synthesis of odd-chained n-alcohols and/or carboxylic acids.

In one embodiment, the primers may be one other than acetyl-CoA or propionyl-CoA, although acetyl-CoA may condense with the primer, acting as an extender unit, to add two carbon units thereto. In another embodiment, these primers in combination with different termination enzymes lead to the synthesis of other products.

In one embodiment, the disclosure describes the one or more termination enzymes are selected from alcohol-forming coenzyme-A thioester reductase, an aldehyde-forming CoA thioester reductase, an alcohol dehydrogenase, a thioesterase, an acyl-CoA:acetyl-CoA transferase, a phosphotransacylase and a carboxylate kinase; aldehyde ferredoxin oxidoreductase; an aldehyde-forming CoA thioester reductase, an aldehyde decarbonylase, alcohol dehydrogenase; aldehyde dehydrogenase, an acyl-CoA reductase, or any combination thereof.

In one embodiment, the disclosure describes operation of multiple turns of a reversal of the beta oxidation cycle, requires the condensation of the acyl-CoA generated from a turn(s) of the cycle with an additional acetyl-CoA molecule to lengthen the acyl-CoA by two carbons each cycle turn. In another embodiment, the initiation and extension of multiple cycle turns requires the use of a thiolase(s) with specificity for longer chain acyl-CoA molecules combined with other pathway enzymes capable of acting on pathway intermediates of increasing carbon number.

While the inventors have demonstrated the efficacy of the disclosure in Clostridium autoethanogenum, the disclosure is applicable to the wider group of anaerobic acetogenic microorganisms and fermentation on substrates comprising CO and/or CO₂, as discussed above and further herein.

Unless otherwise defined, the following terms as used throughout this specification are defined as follows:

The terms “increasing the efficiency,” “increased efficiency,” and the like, when used in relation to a fermentation process, include, but are not limited to, increasing one or more of the rate of growth of microorganisms catalyzing the fermentation, the growth and/or product production rate at elevated product concentrations, increasing the volume of desired product produced per volume of substrate consumed, increasing the rate of production or level of production of the desired product, increasing the relative proportion of the desired product produced compared with other by-products of the fermentation, decreasing the amount of water consumed by the process, and decreasing the amount of energy utilized by the process.

The term “fermentation” should be interpreted as a metabolic process that produces chemical changes in a substrate. For example, a fermentation process receives one or more substrates and produces one or more products through utilization of one or more microorganisms. The term “fermentation,” “gas fermentation” and the like should be interpreted as the process which receives one or more substrate, such as syngas produced by gasification and produces one or more product through the utilization of one or more C1-fixing microorganism. Preferably the fermentation process includes the use of one or more bioreactor. The fermentation process may be described as either “batch” or “continuous”. “Batch fermentation” is used to describe a fermentation process where the bioreactor is filled with raw material, e.g. the carbon source, along with microorganisms, where the products remain in the bioreactor until fermentation is completed. In a “batch” process, after fermentation is completed, the products are extracted, and the bioreactor is cleaned before the next “batch” is started. “Continuous fermentation” is used to describe a fermentation process where the fermentation process is extended for longer periods of time, and product and/or metabolite is extracted during fermentation. Preferably the fermentation process is continuous.

The term “non-naturally occurring” when used in reference to a microorganism is intended to mean that the microorganism has at least one genetic modification not found in a naturally occurring strain of the referenced species, including wild-type strains of the referenced species. Non-naturally occurring microorganisms are typically developed in a laboratory or research facility.

The terms “genetic modification,” “genetic alteration,” or “genetic engineering” broadly refer to manipulation of the genome or nucleic acids of a microorganism by the hand of man. Likewise, the terms “genetically modified,” “genetically altered,” or “genetically engineered” refers to a microorganism containing such a genetic modification, genetic alteration, or genetic engineering. These terms may be used to differentiate a lab-generated microorganism from a naturally-occurring microorganism. Methods of genetic modification of include, for example, heterologous gene expression, gene or promoter insertion or deletion, nucleic acid mutation, altered gene expression or inactivation, enzyme engineering, directed evolution, knowledge-based design, random mutagenesis methods, gene shuffling, and codon optimization.

Metabolic engineering of microorganisms, such as Clostridia, can tremendously expand their ability to produce many important fuel and chemical molecules other than native metabolites, such as ethanol. However, until recently, Clostridia were considered genetically intractable and therefore generally off limits to extensive metabolic engineering efforts. In recent years several different methods for genome engineering for Clostridia have been developed including intron-based methods (ClosTron) (Kuehne, Strain Eng: Methods and Protocols, 389-407, 2011), allelic exchange methods (ACE) (Heap, Nucl Acids Res, 40: e59, 2012; Ng, PLoS One, 8: e56051, 2013), Triple Cross (Liew, Frontiers Microbiol, 7: 694, 2016), methods mediated through I-SceI (Zhang, Journal Microbiol Methods, 108: 49-60, 2015), MazF (Al-Hinai, Appl Environ Microbiol, 78: 8112-8121, 2012), or others (Argyros, Appl Environ Microbiol, 77: 8288-8294, 2011), Cre-Lox (Ueki, mBio, 5: e01636-01614, 2014), and CRISPR/Cas9 (Nagaraju, Biotechnol Biofuels, 9: 219, 2016). However, it remains extremely challenging to iteratively introduce more than a few genetic changes, due to slow and laborious cycling times and limitations on the transferability of these genetic techniques across species. Furthermore, we do not yet sufficiently understand C1 metabolism in Clostridia to reliably predict modifications that will maximize C1 uptake, conversion, and carbon/energy/redox flows towards product synthesis. Accordingly, introduction of target pathways in Clostridia remains a tedious and time-consuming process.

“Recombinant” indicates that a nucleic acid, protein, or microorganism is the product of genetic modification, engineering, or recombination. Generally, the term “recombinant” refers to a nucleic acid, protein, or microorganism that contains or is encoded by genetic material derived from multiple sources, such as two or more different strains or species of microorganisms.

“Wild type” refers to the typical form of an organism, strain, gene, or characteristic as it occurs in nature, as distinguished from mutant or variant forms.

“Endogenous” refers to a nucleic acid or protein that is present or expressed in the wild-type or parental microorganism from which the microorganism of the disclosure is derived. For example, an endogenous gene is a gene that is natively present in the wild-type or parental microorganism from which the microorganism of the disclosure is derived. In one embodiment, the expression of an endogenous gene may be controlled by an exogenous regulatory element, such as an exogenous promoter.

“Exogenous” refers to a nucleic acid or protein that originates outside the microorganism of the disclosure. For example, an exogenous gene or enzyme may be artificially or recombinantly created and introduced to or expressed in the microorganism of the disclosure. An exogenous gene or enzyme may also be isolated from a heterologous microorganism and introduced to or expressed in the microorganism of the disclosure. Exogenous nucleic acids may be adapted to integrate into the genome of the microorganism of the disclosure or to remain in an extra-chromosomal state in the microorganism of the disclosure, for example, in a plasmid.

“Heterologous” refers to a nucleic acid or protein that is not present in the wild-type or parental microorganism from which the microorganism of the disclosure is derived. For example, a heterologous gene or enzyme may be derived from a different strain or species and introduced to or expressed in the microorganism of the disclosure. The heterologous gene or enzyme may be introduced to or expressed in the microorganism of the disclosure in the form in which it occurs in the different strain or species. Alternatively, the heterologous gene or enzyme may be modified in some way, e.g., by codon-optimizing it for expression in the microorganism of the disclosure or by engineering it to alter function, such as to reverse the direction of enzyme activity or to alter substrate specificity.

The terms “polynucleotide,” “nucleotide,” “nucleotide sequence,” “nucleic acid,” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides or nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene products.”

The terms “polypeptide”, “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein, the term “amino acid” includes natural and/or unnatural or synthetic amino acids, including glycine and both the D or L optical isomers, and amino acid analogs and peptidomimetics.

“Enzyme activity,” or simply “activity,” refers broadly to enzymatic activity, including, but not limited, to the activity of an enzyme, the amount of an enzyme, or the availability of an enzyme to catalyze a reaction. Accordingly, “increasing” enzyme activity includes increasing the activity of an enzyme, increasing the amount of an enzyme, or increasing the availability of an enzyme to catalyze a reaction. Similarly, “decreasing” enzyme activity includes decreasing the activity of an enzyme, decreasing the amount of an enzyme, or decreasing the availability of an enzyme to catalyze a reaction.

“Mutated” refers to a nucleic acid or protein that has been modified in the microorganism of the disclosure compared to the wild-type or parental microorganism from which the microorganism of the disclosure is derived. In one embodiment, the mutation may be a deletion, insertion, or substitution in a gene encoding an enzyme. In another embodiment, the mutation may be a deletion, insertion, or substitution of one or more amino acids in an enzyme.

In particular, a “disruptive mutation” is a mutation that reduces or eliminates (i.e., “disrupts”) the expression or activity of a gene or enzyme. The disruptive mutation may partially inactivate, fully inactivate, or delete the gene or enzyme. The disruptive mutation may be any mutation that reduces, prevents, or blocks the biosynthesis of a product produced by an enzyme. The disruptive mutation may be a knockout (KO) mutation. The disruption may also be a knockdown (KD) mutation that reduces, but does not entirely eliminate, the expression or activity of a gene, protein, or enzyme. While KOs are generally effective in increasing product yields, they sometimes come with the penalty of growth defects or genetic instabilities that outweigh the benefits, particularly for non-growth coupled products. The disruptive mutation may include, for example, a mutation in a gene encoding an enzyme, a mutation in a genetic regulatory element involved in the expression of a gene encoding an enzyme, the introduction of a nucleic acid which produces a protein that reduces or inhibits the activity of an enzyme, or the introduction of a nucleic acid (e.g., antisense RNA, siRNA, CRISPR) or protein which inhibits the expression of an enzyme. The disruptive mutation may be introduced using any method known in the art.

Introduction of a disruptive mutation results in a microorganism of the disclosure that produces no target product or substantially no target product or a reduced amount of target product compared to the parental microorganism from which the microorganism of the disclosure is derived. For example, the microorganism of the disclosure may produce no target product or at least about 1%, 3%, 5%, 10%, 20%, 30%, 40%, 50%, 60%, 70%, 80%, 90%, or 95% less target product than the parental microorganism. For example, the microorganism of the disclosure may produce less than about 0.001, 0.01, 0.10, 0.30, 0.50, or 1.0 g/L target product.

“Codon optimization” refers to the mutation of a nucleic acid, such as a gene, for optimized or improved translation of the nucleic acid in a particular strain or species. Codon optimization may result in faster translation rates or translation accuracy. In an embodiment, the genes of the disclosure are codon optimized for expression in Clostridium, particularly Clostridium autoethanogenum, Clostridium ljungdahlii, or Clostridium ragsdalei. In a further embodiment, the genes of the disclosure are codon optimized for expression in Clostridium autoethanogenum LZ1561, which is deposited under DSMZ accession number DSM23693.

“Overexpressed” refers to an increase in expression of a nucleic acid or protein in the microorganism of the disclosure compared to the wild-type or parental microorganism from which the microorganism of the disclosure is derived. Overexpression may be achieved by any means known in the art, including modifying gene copy number, gene transcription rate, gene translation rate, or enzyme degradation rate.

The term “variants” includes nucleic acids and proteins whose sequence varies from the sequence of a reference nucleic acid and protein, such as a sequence of a reference nucleic acid and protein disclosed in the prior art or exemplified herein. The disclosure may be practiced using variant nucleic acids or proteins that perform substantially the same function as the reference nucleic acid or protein. For example, a variant protein may perform substantially the same function or catalyze substantially the same reaction as a reference protein. A variant gene may encode the same or substantially the same protein as a reference gene. A variant promoter may have substantially the same ability to promote the expression of one or more genes as a reference promoter.

Such nucleic acids or proteins may be referred to herein as “functionally equivalent variants.” By way of example, functionally equivalent variants of a nucleic acid may include allelic variants, fragments of a gene, mutated genes, polymorphisms, and the like. Homologous genes from other microorganisms are also examples of functionally equivalent variants. These include homologous genes in species such as Clostridium acetobutylicum, Clostridium beijerinckii, or Clostridium ljungdahlii, the details of which are publicly available on websites such as Genbank or NCBI. Functionally equivalent variants also include nucleic acids whose sequence varies as a result of codon optimization for a particular microorganism. A functionally equivalent variant of a nucleic acid will preferably have at least approximately 70%, approximately 80%, approximately 85%, approximately 90%, approximately 95%, approximately 98%, or greater nucleic acid sequence identity (percent homology) with the referenced nucleic acid. A functionally equivalent variant of a protein will preferably have at least approximately 70%, approximately 80%, approximately 85%, approximately 90%, approximately 95%, approximately 98%, or greater amino acid identity (percent homology) with the referenced protein. The functional equivalence of a variant nucleic acid or protein may be evaluated using any method known in the art.

“Complementarity” refers to the ability of a nucleic acid to form hydrogen bond(s) with another nucleic acid sequence by either traditional Watson-Crick or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Non-limiting examples of stringent conditions are well known in the art (e.g., Tijssen, Laboratory techniques in biochemistry and molecular biology-hybridization with nucleic acid probes, Second Chapter “Overview of principles of hybridization and the strategy of nucleic acid probe assay,” Elsevier, N.Y, 1993).

“Hybridization” refers to a reaction in which one or more polynucleotides react to form a complex that is stabilized via hydrogen bonding between the bases of the nucleotide residues. The hydrogen bonding may occur by Watson Crick base pairing, Hoogsteen binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

Nucleic acids may be delivered to a microorganism of the disclosure using any method known in the art. For example, nucleic acids may be delivered as naked nucleic acids or may be formulated with one or more agents, such as liposomes. The nucleic acids may be DNA, RNA, cDNA, or combinations thereof, as is appropriate. Restriction inhibitors may be used in certain embodiments. Additional vectors may include plasmids, viruses, bacteriophages, cosmids, and artificial chromosomes. In an embodiment, nucleic acids are delivered to the microorganism of the disclosure using a plasmid. By way of example, transformation (including transduction or transfection) may be achieved by electroporation, ultrasonication, polyethylene glycol-mediated transformation, chemical or natural competence, protoplast transformation, prophage induction, or conjugation. In certain embodiments having active restriction enzyme systems, it may be necessary to methylate a nucleic acid before introduction of the nucleic acid into a microorganism.

Furthermore, nucleic acids may be designed to comprise a regulatory element, such as a promoter, to increase or otherwise control expression of a particular nucleic acid. The promoter may be a constitutive promoter or an inducible promoter. Ideally, the promoter is a Wood-Ljungdahl pathway promoter, a ferredoxin promoter, a pyruvate:ferredoxin oxidoreductase promoter, an Rnf complex operon promoter, an ATP synthase operon promoter, or a phosphotransacetylase/acetate kinase operon promoter.

A “primer” is an initiator or starter molecule that is or becomes charged with CoA, and then condenses with another molecule, for example, acetyl-CoA, in the reverse beta oxidation cycle, thus making the primer longer by two carbons. Such molecules include, but are not limited to, oxalyl-CoA, acetyl-CoA, malonyl CoA, succinyl-CoA, hydroxyacyl-CoA, 3-hydroxyproprionyl-CoA, 4-hydroxybutyryl-CoA, 2-aminoacetyl-CoA, 3-aminopropionyl-CoA, 4-aminobutyryl-CoA, isobutyryl-CoA, 3-methyl-butyryl-CoA, 2-hydroxyproprionyl-CoA, 3-hydroxybutyryl-CoA, 2-aminoproprionyl-CoA, propionyl-CoA, and valeryl-CoA.

A “termination” enzyme is meant as enzymes that catalyze reaction taking reverse beta oxidation intermediates out of the pathway cycle, thus “terminating” the running of the cycle. Termination enzymes include, but are not limited to, alcohol-forming coenzyme-A thioester reductase, an aldehyde-forming CoA thioester reductase, an alcohol dehydrogenase, a thioesterase, an acyl-CoA:acetyl-CoA transferase, a phosphotransacylase and a carboxylate kinase; aldehyde ferredoxin oxidoreductase; an aldehyde-forming CoA thioester reductase, an aldehyde decarbonylase, alcohol dehydrogenase; aldehyde dehydrogenase, and an acyl-CoA reductase.

A “microorganism” is a microscopic organism, especially a bacterium, archaeon, virus, or fungus. The microorganism of the disclosure is typically a bacterium. As used herein, recitation of “microorganism” should be taken to encompass “bacterium.”

A “parental microorganism” is a microorganism used to generate a microorganism of the disclosure. The parental microorganism may be a naturally-occurring microorganism (i.e., a wild-type microorganism) or a microorganism that has been previously modified (i.e., a mutant or recombinant microorganism). The microorganism of the disclosure may be modified to express or overexpress one or more enzymes that were not expressed or overexpressed in the parental microorganism. Similarly, the microorganism of the disclosure may be modified to contain one or more genes that were not contained by the parental microorganism. The microorganism of the disclosure may also be modified to not express or to express lower amounts of one or more enzymes that were expressed in the parental microorganism. In one embodiment, the parental microorganism is Clostridium autoethanogenum, Clostridium ljungdahlii, or Clostridium ragsdalei. In an embodiment, the parental microorganism is Clostridium autoethanogenum LZ1561, which was deposited with Deutsche Sammlung von Mikroorganismen and Zellkulturen GmbH (DSMZ) located at Inhoffenstraβe 7B, D-38124 Braunschweig, Germany on Jun. 7, 2010, under the terms of the Budapest Treaty and accorded accession number DSM23693. This strain is described in International Patent Application No. PCT/NZ2011/000144, which published as WO 2012/015317.

The term “derived from” indicates that a nucleic acid, protein, or microorganism is modified or adapted from a different (e.g., a parental or wild-type) nucleic acid, protein, or microorganism, so as to produce a new nucleic acid, protein, or microorganism. Such modifications or adaptations typically include insertion, deletion, mutation, or substitution of nucleic acids or genes. Generally, the microorganism of the disclosure is derived from a parental microorganism. In one embodiment, the microorganism of the disclosure is derived from Clostridium autoethanogenum, Clostridium ljungdahlii, or Clostridium ragsdalei. In an embodiment, the microorganism of the disclosure is derived from Clostridium autoethanogenum LZ1561, which is deposited under DSMZ accession number DSM23693.

The microorganism of the disclosure may be further classified based on functional characteristics. For example, the microorganism of the disclosure may be or may be derived from a C1-fixing microorganism, an anaerobe, an acetogen, an ethanologen, a carboxydotroph, and/or a methanotroph. Table 1 provides a representative list of microorganisms and identifies their functional characteristics.

TABLE 1

| | Wood-Ljungdahl | C1-fixing | Anaerobe | Acetogen | Ethanologen | Autotroph | Carboxydotroph |
|---|---|---|---|---|---|---|---|
| Acetobacterium woodii | + | + | + | + | +/− ¹ | + | − |
| Alkalibaculum bacchii | + | + | + | + | + | + | + |
| Blautia producta | + | + | + | + | − | + | + |
| Butyribacterium methylotrophicum | + | + | + | + | + | + | + |
| Clostridium aceticum | + | + | + | + | − | + | + |
| Clostridium autoethanogenum | + | + | + | + | + | + | + |
| Clostridium carboxidivorans | + | + | + | + | + | + | + |
| Clostridium coskatii | + | + | + | + | + | + | + |
| Clostridium drakei | + | + | + | + | − | + | + |
| Clostridium formicoaceticum | + | + | + | + | − | + | + |
| Clostridium ljungdahlii | + | + | + | + | + | + | + |
| Clostridium magnum | + | + | + | + | − | + | +/− ² |
| Clostridium ragsdalei | + | + | + | + | + | + | + |
| Clostridium scatologenes | + | + | + | + | − | + | + |
| Eubacterium limosum | + | + | + | + | − | + | + |
| Moorella thermautotrophica | + | + | + | + | + | + | + |
| Moorella thermoacetica (formerly | + | + | + | + | − ³ | + | + |
| Clostridium thermoaceticum) | | | | | | | |
| Oxobacter pfennigii | + | + | + | + | − | + | + |
| Sporomusa ovata | + | + | + | + | − | + | +/− ⁴ |
| Sporomusa silvacetica | + | + | + | + | − | + | +/− ⁵ |
| Sporomusa sphaeroides | + | + | + | + | − | + | +/−⁶ |
| Thermoanaerobacter kivui | + | + | + | + | − | + | − |

¹ Acetobacterium woodii can produce ethanol from fructose, but not from gas.
² It has not been investigated whether Clostridium magnum can grow on CO.
³ One strain of Moorella thermoacetica, Moorella sp. HUC22-1, has been reported to produce ethanol from gas.
⁴ It has not been investigated whether Sporomusa ovata can grow on CO.
⁵ It has not been investigated whether Sporomusa silvacetica can grow on CO.
⁶ It has not been investigated whether Sporomusa sphaeroides can grow on CO.

“Wood-Ljungdahl” refers to the Wood-Ljungdahl pathway of carbon fixation as described, e.g., by Ragsdale, Biochim Biophys Acta, 1784: 1873-1898, 2008. “Wood-Ljungdahl microorganisms” refers, predictably, to microorganisms containing the Wood-Ljungdahl pathway. Generally, the microorganism of the disclosure contains a native Wood-Ljungdahl pathway. Herein, a Wood-Ljungdahl pathway may be a native, unmodified Wood-Ljungdahl pathway or it may be a Wood-Ljungdahl pathway with some degree of genetic modification (e.g., overexpression, heterologous expression, knockout, etc.) so long as it still functions to convert CO, CO₂, and/or H₂ to acetyl-CoA.

“C1” refers to a one-carbon molecule, for example, CO, CO₂, CH₄, or CH₃OH. “C1-oxygenate” refers to a one-carbon molecule that also comprises at least one oxygen atom, for example, CO, CO₂, or CH₃OH. “C1-carbon source” refers a one carbon-molecule that serves as a partial or sole carbon source for the microorganism of the disclosure. For example, a C1-carbon source may comprise one or more of CO, CO₂, CH₄, CH₃OH, or CH₂O₂. Preferably, the C1-carbon source comprises one or both of CO and CO₂. A “C1-fixing microorganism” is a microorganism that has the ability to produce one or more products from a C1 carbon source. Typically, the microorganism of the disclosure is a C1-fixing bacterium. In an embodiment, the microorganism of the disclosure is derived from a C1-fixing microorganism identified in Table 1.

An “anaerobe” is a microorganism that does not require oxygen for growth. An anaerobe may react negatively or even die if oxygen is present above a certain threshold. However, some anaerobes are capable of tolerating low levels of oxygen (e.g., 0.000001-5% oxygen). Typically, the microorganism of the disclosure is an anaerobe. In an embodiment, the microorganism of the disclosure is derived from an anaerobe identified in Table 1.

“Acetogens” are obligately anaerobic bacteria that use the Wood-Ljungdahl pathway as their main mechanism for energy conservation and for synthesis of acetyl-CoA and acetyl-CoA-derived products, such as acetate (Ragsdale, Biochim Biophys Acta, 1784: 1873-1898, 2008). In particular, acetogens use the Wood-Ljungdahl pathway as a (1) mechanism for the reductive synthesis of acetyl-CoA from CO₂, (2) terminal electron-accepting, energy conserving process, (3) mechanism for the fixation (assimilation) of CO₂ in the synthesis of cell carbon (Drake, Acetogenic Prokaryotes, In: The Prokaryotes, 3rd edition, p. 354, New York, N.Y., 2006). All naturally occurring acetogens are C1-fixing, anaerobic, autotrophic, and non-methanotrophic. Typically, the microorganism of the disclosure is an acetogen. In an embodiment, the microorganism of the disclosure is derived from an acetogen identified in Table 1.

An “ethanologen” is a microorganism that produces or is capable of producing ethanol. Typically, the microorganism of the disclosure is an ethanologen. In an embodiment, the microorganism of the disclosure is derived from an ethanologen identified in Table 1.

An “autotroph” is a microorganism capable of growing in the absence of organic carbon. Instead, autotrophs use inorganic carbon sources, such as CO and/or CO₂. Typically, the microorganism of the disclosure is an autotroph. In an embodiment, the microorganism of the disclosure is derived from an autotroph identified in Table 1.

A “carboxydotroph” is a microorganism capable of utilizing CO as a sole source of carbon and energy. Typically, the microorganism of the disclosure is a carboxydotroph. In an embodiment, the microorganism of the disclosure is derived from a carboxydotroph identified in Table 1.

A “methanotroph” is a microorganism capable of utilizing methane as a sole source of carbon and energy. In certain embodiments, the microorganism of the disclosure is a methanotroph or is derived from a methanotroph. In other embodiments, the microorganism of the disclosure is not a methanotroph or is not derived from a methanotroph.

More broadly, the microorganism of the disclosure may be derived from any genus or species identified in Table 1. For example, the microorganism may be a member of a genus selected from the group consisting of Acetobacterium, Alkalibaculum, Blautia, Butyribacterium, Clostridium, Eubacterium, Moorella, Oxobacter, Sporomusa, and Thermoanaerobacter. In particular, the microorganism may be derived from a parental bacterium selected from the group consisting of Acetobacterium woodii, Alkalibaculum bacchii, Blautia producta, Butyribacterium methylotrophicum, Clostridium aceticum, Clostridium autoethanogenum, Clostridium carboxidivorans, Clostridium coskatii, Clostridium drakei, Clostridium formicoaceticum, Clostridium ljungdahlii, Clostridium magnum, Clostridium ragsdalei, Clostridium scatologenes, Eubacterium limosum, Moorella thermautotrophica, Moorella thermoacetica, Oxobacter pfennigii, Sporomusa ovata, Sporomusa silvacetica, Sporomusa sphaeroides, and Thermoanaerobacter kivui.

In some embodiments, however, the microorganism of the invention is a microorganism other than Clostridium autoethanogenum, Clostridium ljungdahlii, or Clostridium ragsdalei. For example, the microorganism may be selected from the group consisting of Escherichia coli, Saccharomyces cerevisiae, Clostridium acetobutylicum, Clostridium beijerinckii, Clostridium saccharbutyricum, Clostridium saccharoperbutylacetonicum, Clostridium butyricum, Clostridium diolis, Clostridium kluyveri, Clostridium pasteurianum, Clostridium novyi, Clostridium difficile, Clostridium thermocellum, Clostridium cellulolyticum, Clostridium cellulovorans, Clostridium phytofermentans, Lactococcus lactis, Bacillus subtilis, Bacillus licheniformis, Zymomonas mobilis, Klebsiella oxytoca, Klebsiella pneumonia, Corynebacterium glutamicum, Trichoderma reesei, Cupriavidus necator, Pseudomonas putida, Lactobacillus plantarum, and Methylobacterium extorquens.

In an embodiment, the microorganism of the disclosure is derived from the cluster of Clostridia comprising the species Clostridium autoethanogenum, Clostridium ljungdahlii, and Clostridium ragsdalei. These species were first reported and characterized by Abrini, Arch Microbiol, 161: 345-351, 1994 (Clostridium autoethanogenum), Tanner, Int J System Bacteriol, 43: 232-236, 1993 (Clostridium ljungdahlii), and Huhnke, WO 2008/028055 (Clostridium ragsdalei).

These three species have many similarities. In particular, these species are all C1 fixing, anaerobic, acetogenic, ethanologenic, and carboxydotrophic members of the genus Clostridium. These species have similar genotypes and phenotypes and modes of energy conservation and fermentative metabolism. Moreover, these species are clustered in clostridial rRNA homology group I with 16S rRNA DNA that is more than 99% identical, have a DNA G+C content of about 22-30 mol %, are gram-positive, have similar morphology and size (logarithmic growing cells between 0.5-0.7×3-5 μm), are mesophilic (grow optimally at 30-37° C.), have similar pH ranges of about 4-7.5 (with an optimal pH of about 5.5-6), lack cytochromes, and conserve energy via an Rnf complex. Also, reduction of carboxylic acids into their corresponding alcohols has been shown in these species (Perez, Biotechnol Bioeng, 110:1066-1077, 2012). Importantly, these species also all show strong autotrophic growth on CO-containing gases, produce ethanol and acetate (or acetic acid) as main fermentation products, and produce small amounts of 2,3-butanediol and lactic acid under certain conditions.

However, these three species also have a number of differences. These species were isolated from different sources: Clostridium autoethanogenum from rabbit gut, Clostridium ljungdahlii from chicken yard waste, and Clostridium ragsdalei from freshwater sediment. These species differ in utilization of various sugars (e.g., rhamnose, arabinose), acids (e.g., gluconate, citrate), amino acids (e.g., arginine, histidine), and other substrates (e.g., betaine, butanol). Moreover, these species differ in auxotrophy to certain vitamins (e.g., thiamine, biotin). These species have differences in nucleic and amino acid sequences of Wood-Ljungdahl pathway genes and proteins, although the general organization and number of these genes and proteins has been found to be the same in all species (Köpke, Curr Opin Biotechnol, 22: 320-325, 2011).

Thus, in summary, many of the characteristics of Clostridium autoethanogenum, Clostridium ljungdahlii, or Clostridium ragsdalei are not specific to that species, but are rather general characteristics for this cluster of C1 fixing, anaerobic, acetogenic, ethanologenic, and carboxydotrophic members of the genus Clostridium. However, since these species are, in fact, distinct, the genetic modification or manipulation of one of these species may not have an identical effect in another of these species. For instance, differences in growth, performance, or product production may be observed.

The microorganism of the disclosure may also be derived from an isolate or mutant of Clostridium autoethanogenum, Clostridium ljungdahlii, or Clostridium ragsdalei. Isolates and mutants of Clostridium autoethanogenum include JA1-1 (DSM10061) (Abrini, Arch Microbiol, 161: 345-351, 1994), LZ1560 (DSM19630) (WO 2009/064200), and LZ1561 (DSM23693) (WO 2012/015317). Isolates and mutants of Clostridium ljungdahlii include ATCC 49587 (Tanner, Int J Syst Bacteriol, 43: 232-236, 1993), PETCT (DSM13528, ATCC 55383), ERI-2 (ATCC 55380) (U.S. Pat. No. 5,593,886), C-01 (ATCC 55988) (U.S. Pat. No. 6,368,819), 0-52 (ATCC 55989) (U.S. Pat. No. 6,368,819), and OTA-1 (Tirado-Acevedo, Production of bioethanol from synthesis gas using Clostridium ljungdahlii, PhD thesis, North Carolina State University, 2010). Isolates and mutants of Clostridium ragsdalei include PI 1 (ATCC BAA-622, ATCC PTA-7826) (WO 2008/028055).

“Substrate” refers to a carbon and/or energy source for the microorganism of the disclosure. Typically, the substrate is gaseous and comprises a C1-carbon source, for example, CO, CO₂, and/or CH₄. Preferably, the substrate comprises a C1-carbon source of CO or CO+CO₂. The substrate may further comprise other non-carbon components, such as H₂, N₂, or electrons.

The substrate generally comprises at least some amount of CO, such as about 1, 2, 5, 10, 20, 30, 40, 50, 60, 70, 80, 90, or 100 mol % CO. The substrate may comprise a range of CO, such as about 20-80, 30-70, or 40-60 mol % CO. Preferably, the substrate comprises about 40-70 mol % CO (e.g., steel mill or blast furnace gas), about 20-30 mol % CO (e.g., basic oxygen furnace gas), or about 15-45 mol % CO (e.g., syngas). In some embodiments, the substrate may comprise a relatively low amount of CO, such as about 1-10 or 1-20 mol % CO. The microorganism of the disclosure typically converts at least a portion of the CO in the substrate to a product. In some embodiments, the substrate comprises no or substantially no (<1 mol %) CO.

The substrate may comprise some amount of H₂. For example, the substrate may comprise about 1, 2, 5, 10, 15, 20, or 30 mol % H₂. In some embodiments, the substrate may comprise a relatively high amount of H₂, such as about 60, 70, 80, or 90 mol % H₂. In further embodiments, the substrate comprises no or substantially no (<1 mol %) H₂.

The substrate may comprise some amount of CO₂. For example, the substrate may comprise about 1-80 or 1-30 mol % CO₂. In some embodiments, the substrate may comprise less than about 20, 15, 10, or 5 mol % CO₂. In another embodiment, the substrate comprises no or substantially no (<1 mol %) CO₂.

Although the substrate is typically gaseous, the substrate may also be provided in alternative forms. For example, the substrate may be dissolved in a liquid saturated with a CO-containing gas using a microbubble dispersion generator. By way of further example, the substrate may be adsorbed onto a solid support.

The substrate and/or C1-carbon source may be a waste gas obtained as a byproduct of an industrial process or from some other source, such as from automobile exhaust fumes or biomass gasification. In certain embodiments, the industrial process is selected from the group consisting of ferrous metal products manufacturing, such as a steel mill manufacturing, non-ferrous products manufacturing, petroleum refining, coal gasification, electric power production, carbon black production, ammonia production, methanol production, and coke manufacturing. In these embodiments, the substrate and/or C1-carbon source may be captured from the industrial process before it is emitted into the atmosphere, using any convenient method.

The substrate and/or C1-carbon source may be syngas, such as syngas obtained by gasification of coal or refinery residues, gasification of biomass or lignocellulosic material, or reforming of natural gas. In another embodiment, the syngas may be obtained from the gasification of municipal solid waste or industrial solid waste. The substrate and/or C1-carbon source may be derived from pyrolysis with or without subsequent partial oxidation of the pyrolysis oil.

The composition of the substrate may have a significant impact on the efficiency and/or cost of the reaction. For example, the presence of oxygen (O₂) may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

In particular embodiments, the presence of hydrogen results in an improved overall efficiency of the fermentation process.

Syngas composition can be improved to provide a desired or optimum H₂:CO:CO₂ ratio. The syngas composition may be improved by adjusting the feedstock being fed to the gasification process. The desired H₂:CO:CO₂ ratio is dependent on the desired fermentation product of the fermentation process. For ethanol, the optimum H₂:CO:CO₂ ratio would be:

$$( x ) : ( y ) : ( \frac{x - 2 y}{3} ) ,$$

where x>2y, in order to satisfy the stoichiometry for ethanol production

$$( x ) {H}_{2} + ( y ) CO + ( \frac{x - 2 y}{3} ) {CO}_{2} \to ( \frac{x + y}{6} ) {C}_{2} {H}_{5} OH + ( \frac{x - y}{2} ) {H}_{2} O .$$

Operating the fermentation process in the presence of hydrogen has the added benefit of reducing the amount of CO₂ produced by the fermentation process. For example, a gaseous substrate comprising minimal H₂ will typically produce ethanol and CO₂ by the following stoichiometry [6 CO+3H₂O→C₂H₅OH+4 CO₂]. As the amount of hydrogen utilized by the C1-fixing bacterium increases, the amount of CO₂ produced decreases [e.g., 2 CO+4H₂→C₂H₅OH+H₂O].

When CO is the sole carbon and energy source for ethanol production, a portion of the carbon is lost to CO₂ as follows:

6CO+3H₂O→C₂H₅OH+→CO₂(ΔG°=−224.90 kJ/mol ethanol)

As the amount of H₂ available in the substrate increases, the amount of CO₂ produced decreases. At a stoichiometric ratio of 2:1 (H₂:CO), CO₂ production is completely avoided.

5CO+1H₂+2H₂O→1C₂H₅OH+3CO₂(ΔG°=−204.80 kJ/mol ethanol)

4CO+2H₂+1H₂O→1C₂H₅OH+2CO₂(ΔG°=−184.70 kJ/mol ethanol)

3CO+3H₂→1C₂H₅OH+1CO₂(ΔG°=−164.60 kJ/mol ethanol)

“Stream” refers to any substrate which is capable of being passed, for example, from one process to another, from one module to another, and/or from one process to a carbon capture means.

“Reactants” as used herein refer to a substance that takes part in and undergoes change during a chemical reaction. In particular embodiments, the reactants include but are not limited to CO and/or H₂.

“Microbe inhibitors” as used herein refer to one or more constituent that slows down or prevents a particular chemical reaction or another process including the microbe. In particular embodiments, the microbe inhibitors include, but are not limited to, oxygen (02), hydrogen cyanide (HCN), acetylene (C₂H₂), and BTEX (benzene, toluene, ethylbenzene, xylene).

“Catalyst inhibitor”, “adsorbent inhibitor”, and the like, as used herein, refer to one or more substance that decreases the rate of, or prevents, a chemical reaction. In particular embodiments, the catalyst and/or adsorbent inhibitors may include but are not limited to, hydrogen sulfide (H₂S) and carbonyl sulfide (COS).

“Removal process”, “removal module”, “clean-up module”, and the like includes technologies that are capable of either converting and/or removing microbe inhibitors and/or catalyst inhibitors from the gas stream. In particular embodiments, catalyst inhibitors must be removed by an upstream removal module in order to prevent inhibition of one or more catalyst in a downstream removal module.

The term “constituents”, “contaminants”, and the like, as used herein, refers to the microbe inhibitors, and/or catalyst inhibitors that may be found in the gas stream. In particular embodiments, the constituents include, but are not limited to, sulphur compounds, aromatic compounds, alkynes, alkenes, alkanes, olefins, nitrogen compounds, phosphorous-containing compounds, particulate matter, solids, oxygen, halogenated compounds, silicon-containing compounds, carbonyls, metals, alcohols, esters, ketones, peroxides, aldehydes, ethers, and tars.

The term “treated gas”, “treated stream” and the like refers to the gas stream that has been passed through at least one removal module and has had one or more constituent removed and/or converted.

The term “desired composition” is used to refer to the desired level and types of components in a substance, such as, for example, of a gas stream, including but not limited to syngas. More particularly, a gas is considered to have a “desired composition” if it contains a particular component (e.g. CO, H₂, and/or CO₂) and/or contains a particular component at a particular proportion and/or does not contain a particular component (e.g. a contaminant harmful to the microorganisms) and/or does not contain a particular component at a particular proportion. More than one component may be considered when determining whether a gas stream has a desired composition.

The composition of the substrate may have a significant impact on the efficiency and/or cost of the reaction. For example, the presence of oxygen (O₂) may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

The term “carbon capture” as used herein refers to the sequestration of carbon compounds including CO₂ and/or CO from a stream comprising CO₂ and/or CO and either:

converting the CO₂ and/or CO into products; or
converting the CO₂ and/or CO into substances suitable for long-term storage; or
trapping the CO₂ and/or CO in substances suitable for long-term storage;
or a combination of these processes.

In certain embodiments, the fermentation is performed in the absence of carbohydrate substrates, such as sugar, starch, lignin, cellulose, or hemicellulose.

The microorganism of the disclosure may be cultured with the gaseous substrate to produce one or more products. For instance, the microorganism of the disclosure may produce or may be engineered to produce ethanol (WO 2007/117157), acetate (WO 2007/117157), 1-butanol (WO 2008/115080, WO 2012/053905, and WO 2017/066498), butyrate (WO 2008/115080), 2,3-butanediol (WO 2009/151342 and WO 2016/094334), lactate (WO 2011/112103), butene (WO 2012/024522), butadiene (WO 2012/024522), methyl ethyl ketone (2-butanone) (WO 2012/024522 and WO 2013/185123), ethylene (WO 2012/026833), acetone (WO 2012/115527), isopropanol (WO 2012/115527), lipids (WO 2013/036147), 3-hydroxypropionate (3-HP) (WO 2013/180581), terpenes, including isoprene (WO 2013/180584), fatty acids (WO 2013/191567), 2-butanol (WO 2013/185123), 1,2-propanediol (WO 2014/036152), 1-propanol (WO 2017/066498), 1-hexanol (WO 2017/066498), 1-octanol (WO 2017/066498), chorismate-derived products (WO 2016/191625), 3-hydroxybutyrate (WO 2017/066498), 1,3-butanediol (WO 2017/066498), 2-hydroxyisobutyrate or 2-hydroxyisobutyric acid (WO 2017/066498), isobutylene (WO 2017/066498), adipic acid (WO 2017/066498), 1,3-hexanediol (WO 2017/066498), 3-methyl-2-butanol (WO 2017/066498), 2-buten-1-ol (WO 2017/066498), isovalerate (WO 2017/066498), isoamyl alcohol (WO 2017/066498), and/or monoethylene glycol (WO 2019/126400) in addition to 2-phenylethanol. In certain embodiments, microbial biomass itself may be considered a product. These products may be further converted to produce at least one component of diesel, jet fuel, and/or gasoline. In certain embodiments, 2-phenylethanol may be used as an ingredient in fragrances, essential oils, flavors, and soaps. Additionally, the microbial biomass may be further processed to produce a single cell protein (SCP).

A “single cell protein” (SCP) refers to a microbial biomass that may be used in protein-rich human and/or animal feeds, often replacing conventional sources of protein supplementation such as soymeal or fishmeal. To produce a single cell protein or other product, the process may comprise additional separation, processing, or treatments steps. For example, the method may comprise sterilizing the microbial biomass, centrifuging the microbial biomass, and/or drying the microbial biomass. In certain embodiments, the microbial biomass is dried using spray drying or paddle drying. The method may also comprise reducing the nucleic acid content of the microbial biomass using any method known in the art, since intake of a diet high in nucleic acid content may result in the accumulation of nucleic acid degradation products and/or gastrointestinal distress. The single cell protein may be suitable for feeding to animals, such as livestock or pets. In particular, the animal feed may be suitable for feeding to one or more beef cattle, dairy cattle, pigs, sheep, goats, horses, mules, donkeys, deer, buffalo/bison, llamas, alpacas, reindeer, camels, bantengs, gayals, yaks, chickens, turkeys, ducks, geese, quail, guinea fowl, squabs/pigeons, fish, shrimp, crustaceans, cats, dogs, and rodents. The composition of the animal feed may be tailored to the nutritional requirements of different animals. Furthermore, the process may comprise blending or combining the microbial biomass with one or more excipients.

An “excipient” may refer to any substance that may be added to the microbial biomass to enhance or alter the form, properties, or nutritional content of the animal feed. For example, the excipient may comprise one or more of a carbohydrate, fiber, fat, protein, vitamin, mineral, water, flavor, sweetener, antioxidant, enzyme, preservative, probiotic, or antibiotic. In some embodiments, the excipient may be hay, straw, silage, grains, oils or fats, or other plant material. The excipient may be any feed ingredient identified in Chiba, Section 18: Diet Formulation and Common Feed Ingredients, Animal Nutrition Handbook, 3rd revision, pages 575-633, 2014.

A “native product” is a product produced by a genetically unmodified microorganism. For example, ethanol, acetate, and 2,3-butanediol are native products of Clostridium autoethanogenum, Clostridium ljungdahlii, and Clostridium ragsdalei. A “non-native product” is a product that is produced by a genetically modified microorganism but is not produced by a genetically unmodified microorganism from which the genetically modified microorganism is derived.

“Selectivity” refers to the ratio of the production of a target product to the production of all fermentation products produced by a microorganism. The microorganism of the disclosure may be engineered to produce products at a certain selectivity or at a minimum selectivity. In one embodiment, a target product account for at least about 5%, 10%, 15%, 20%, 30%, 50%, or 75% of all fermentation products produced by the microorganism of the disclosure. In one embodiment, the target product accounts for at least 10% of all fermentation products produced by the microorganism of the disclosure, such that the microorganism of the disclosure has a selectivity for the target product of at least 10%. In another embodiment, the target product accounts for at least 30% of all fermentation products produced by the microorganism of the disclosure, such that the microorganism of the disclosure has a selectivity for the target product of at least 30%.

“Increasing the efficiency,” “increased efficiency,” and the like include, but are not limited to, increasing growth rate, product production rate or volume, product volume per volume of substrate consumed, or product selectivity. Efficiency may be measured relative to the performance of parental microorganism from which the microorganism of the disclosure is derived.

Typically, the culture is performed in a bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some embodiments, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

The culture is generally maintained in an aqueous culture medium that contains nutrients, vitamins, and/or minerals sufficient to permit growth of the microorganism. Preferably the aqueous culture medium is an anaerobic microbial growth medium, such as a minimal anaerobic microbial growth medium. Suitable media are well known in the art.

The culture/fermentation should desirably be carried out under appropriate conditions for production of the target product. Typically, the culture/fermentation is performed under anaerobic conditions. Reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition. In particular, the rate of introduction of the substrate may be controlled to ensure that the concentration of gas in the liquid phase does not become limiting, since products may be consumed by the culture under gas-limited conditions.

Operating a bioreactor at elevated pressures allows for an increased rate of gas mass transfer from the gas phase to the liquid phase. Accordingly, it is generally preferable to perform the culture/fermentation at pressures higher than atmospheric pressure. Also, since a given gas conversion rate is, in part, a function of the substrate retention time and retention time dictates the required volume of a bioreactor, the use of pressurized systems can greatly reduce the volume of the bioreactor required and, consequently, the capital cost of the culture/fermentation equipment. This, in turn, means that the retention time, defined as the liquid volume in the bioreactor divided by the input gas flow rate, can be reduced when bioreactors are maintained at elevated pressure rather than atmospheric pressure. The optimum reaction conditions will depend partly on the particular microorganism used. However, in general, it is preferable to operate the fermentation at a pressure higher than atmospheric pressure. Also, since a given gas conversion rate is in part a function of substrate retention time and achieving a desired retention time in turn dictates the required volume of a bioreactor, the use of pressurized systems can greatly reduce the volume of the bioreactor required, and consequently the capital cost of the fermentation equipment.

In certain embodiments, the fermentation is performed in the absence of light or in the presence of an amount of light insufficient to meet the energetic requirements of photosynthetic microorganisms. In certain embodiments, the microorganism of the disclosure is a non-photosynthetic microorganism.

As used herein, the terms “fermentation broth” or “broth” refer to the mixture of components in a bioreactor, which includes cells and nutrient media. As used herein, a “separator” is a module that is adapted to receive fermentation broth from a bioreactor and pass the broth through a filter to yield a “retentate” and a “permeate.” The filter may be a membrane, e.g. a cross-flow membrane or a hollow fibre membrane. The term “permeate” is used to refer to substantially soluble components of the broth that pass through the separator. The permeate will typically contain soluble fermentation products, byproducts, and nutrients. The retentate will typically contain cells. As used herein, the term “broth bleed” is used to refer to a portion of the fermentation broth that is removed from a bioreactor and not passed to a separator.

Target products may be separated or purified from a fermentation broth using any method or combination of methods known in the art, including, for example, fractional distillation, evaporation, pervaporation, gas stripping, phase separation, and extractive fermentation, including for example, liquid-liquid extraction. In certain embodiments, target products are recovered from the fermentation broth by continuously removing a portion of the broth from the bioreactor, separating microbial cells from the broth (conveniently by filtration), and recovering one or more target products from the broth. Alcohols and/or acetone may be recovered, for example, by distillation. Acids may be recovered, for example, by adsorption on activated charcoal. Separated microbial cells are preferably recycled back to the bioreactor. The cell-free permeate remaining after target products have been removed is also preferably returned to the bioreactor. Additional nutrients may be added to the cell-free permeate to replenish the medium before it is returned to the bioreactor.

As referred to herein, a “shuttle microorganism” is a microorganism in which a methyltransferase enzyme is expressed and is distinct from the destination microorganism.

As referred to herein, a “destination microorganism” is a microorganism in which the genes included on an expression construct/vector are expressed and is distinct from the shuttle microorganism. This is also called a host microorganism.

The term “main fermentation product” is intended to mean the one fermentation product which is produced in the highest concentration and/or yield. There may be one or more fermentation products. The most prevalent may or may not be the most commercially valuable.

The phrase “substrate comprising carbon monoxide” and like terms should be understood to include any substrate in which carbon monoxide is available to one or more strains of bacteria for growth and/or fermentation, for example.

The phrase “gaseous substrate comprising carbon monoxide” and like phrases and terms includes any gas which contains a level of carbon monoxide. In certain embodiments the substrate contains at least about 20% to about 100% CO by volume, from 20% to 70% CO by volume, from 30% to 60% CO by volume, and from 40% to 55% CO by volume. In particular embodiments, the substrate comprises about 25%, or about 30%, or about 35%, or about 40%, or about 45%, or about 50% CO, or about 55% CO, or about 60% CO by volume.

While it is not necessary for a substrate comprising CO to contain any hydrogen, the presence of H₂ should not be detrimental to product formation in accordance with methods of the disclosure. In particular embodiments, the presence of hydrogen results in an improved overall efficiency of alcohol production. For example, in particular embodiments, the substrate may comprise an approx. 2:1, or 1:1, or 1:2 ratio of H₂:CO. In one embodiment the substrate comprises about 30% or less H₂ by volume, 20% or less H₂ by volume, about 15% or less H₂ by volume or about 10% or less H₂ by volume. In other embodiments, the substrate stream comprises low concentrations of H₂, for example, less than 5%, or less than 4%, or less than 3%, or less than 2%, or less than 1%, or is substantially hydrogen free. The substrate may also contain some CO₂ for example, such as about 1% to about 80% CO₂ by volume, or 1% to about 30% CO₂ by volume. In one embodiment the substrate comprises less than or equal to about 20% CO₂ by volume. In particular embodiments the substrate comprises less than or equal to about 15% CO₂ by volume, less than or equal to about 10% CO₂ by volume, less than or equal to about 5% CO₂ by volume or substantially no CO₂.

The phrase “substrate comprising carbon dioxide” and like terms should be understood to include any substrate in which carbon dioxide is available to one or more strains of bacteria for growth and/or fermentation, for example. Substrates comprising carbon dioxide may further comprise hydrogen and/or carbon monoxide.

The phrase “gaseous substrate comprising carbon dioxide” and like phrases and terms includes any gas which contains a level of carbon dioxide. In certain embodiments the substrate contains at least about 10% to about 60% CO₂ by volume, from 20% to 50% CO₂ by volume, from 30% to 60% CO₂ by volume, and from 40% to 55% CO₂ by volume. In particular embodiments, the substrate comprises about 20%, or about 25%, or about 30%, or about 35%, or about 40%, or about 45%, or about 50% CO, or about 55% CO, or about 60% CO₂ by volume.

Preferably, a substrate comprising CO₂ will also contain a level of CO or H₂. In particular embodiments, the substrate comprises a CO₂:H₂ ratio of at least about 1:1, or at least about 1:2, or at least about 1:3, or at least about 1:4, or at least about 1:5.

In the description which follows, embodiments of the disclosure are described in terms of delivering and fermenting a “gaseous substrate containing CO and/or CO₂.” However, it should be appreciated that the gaseous substrate may be provided in alternative forms. For example, the gaseous substrate containing CO and/or CO₂ may be provided dissolved in a liquid. Essentially, a liquid is saturated with a carbon monoxide containing gas and then that liquid is added to the bioreactor. This may be achieved using standard methodology. By way of example, a microbubble dispersion generator (Hensirisak et. al. Scale-up of microbubble dispersion generator for aerobic fermentation; Applied Biochemistry and Biotechnology Volume 101, Number 3/October 2002) could be used. By way of further example, the gaseous substrate containing CO may be adsorbed onto a solid support. Such alternative methods are encompassed by use of the term “substrate containing CO and/or CO₂” and the like.

In particular embodiments of the disclosure, the CO-containing gaseous substrate (or a gaseous substrate comprising CO₂, or CO and CO₂, or CO₂ and H₂ and CO) is an industrial off or waste gas. “Industrial waste or off gases” should be taken broadly to include any gases comprising CO and/or CO₂ produced by an industrial process and include gases produced as a result of ferrous metal products manufacturing, non-ferrous products manufacturing, petroleum refining processes, gasification of coal, gasification of biomass, electric power production, carbon black production, and coke manufacturing. Further examples may be provided elsewhere herein.

Unless the context requires otherwise, the phrases “fermenting”, “fermentation process” or “fermentation reaction” and the like, as used herein, are intended to encompass both the growth phase and product biosynthesis phase of the process. As will be described further herein, in some embodiments the bioreactor may comprise a first growth reactor and a second fermentation reactor. As such, the addition of metals or compositions to a fermentation reaction should be understood to include addition to either or both of these reactors.

The term “bioreactor” includes a fermentation device consisting of one or more vessels and/or towers or piping arrangement, which includes the Continuous Stirred Tank Reactor (CSTR), Immobilized Cell Reactor (ICR), Trickle Bed Reactor (TBR), Bubble Column, Gas Lift Fermenter, Static Mixer, or other vessel or other device suitable for gas-liquid contact. In some embodiments the bioreactor may comprise a first growth reactor and a second fermentation reactor. As such, when referring to the addition of substrate to the bioreactor or fermentation reaction it should be understood to include addition to either or both of these reactors where appropriate.

It should be appreciated that the disclosure may be practiced using nucleic acids whose sequence varies from the sequences specifically exemplified herein provided they perform substantially the same function. For nucleic acid sequences that encode a protein or peptide this means that the encoded protein or peptide has substantially the same function. For nucleic acid sequences that represent promoter sequences, the variant sequence will have the ability to promote expression of one or more genes. Such nucleic acids may be referred to herein as “functionally equivalent variants.” By way of example, functionally equivalent variants of a nucleic acid include allelic variants, fragments of a gene, genes which include mutations (deletion, insertion, nucleotide substitutions and the like) and/or polymorphisms and the like. Homologous genes from other microorganisms may also be considered as examples of functionally equivalent variants of the sequences specifically exemplified herein.

These include homologous genes in species such as Clostridium ljungdahlii, Chloroflexus aurantiacus, Metallosphaera or Sulfolobus spp, details of which are publicly available on websites such as Genbank or NCBI. The phrase “functionally equivalent variants” should also be taken to include nucleic acids whose sequence varies as a result of codon optimisation for a particular organism. “Functionally equivalent variants” of a nucleic acid herein will preferably have at least approximately 70%, preferably approximately 80%, more preferably approximately 85%, preferably approximately 90%, preferably approximately 95% or greater nucleic acid sequence identity with the nucleic acid identified.

It should also be appreciated that the disclosure may be practiced using polypeptides whose sequence varies from the amino acid sequences specifically exemplified herein. These variants may be referred to herein as “functionally equivalent variants.” A functionally equivalent variant of a protein or a peptide includes those proteins or peptides that share at least 40%, preferably 50%, preferably 60%, preferably 70%, preferably 75%, preferably 80%, preferably 85%, preferably 90%, preferably 95% or greater amino acid identity with the protein or peptide identified and has substantially the same function as the peptide or protein of interest. Such variants include within their scope fragments of a protein or peptide wherein the fragment comprises a truncated form of the polypeptide wherein deletions may be from 1 to 5, to 10, to 15, to 20, to 25 amino acids, and may extend from residue 1 through 25 at either terminus of the polypeptide, and wherein deletions may be of any length within the region; or may be at an internal location. Functionally equivalent variants of the specific polypeptides herein should also be taken to include polypeptides expressed by homologous genes in other species of bacteria, for example as exemplified in the previous paragraph.

The microorganisms of the disclosure may be prepared from a parental microorganism and one or more exogenous nucleic acids using any number of techniques known in the art for producing recombinant microorganisms. By way of example only, transformation (including transduction or transfection) may be achieved by electroporation, ultrasonication, polyethylene glycol-mediated transformation, chemical or natural competence, or conjugation. Suitable transformation techniques are described for example in, Sambrook J, Fritsch E F, Maniatis T: Molecular Cloning: A laboratory Manual, Cold Spring Harbour Laboratory Press, Cold Spring Harbour, 1989.

In certain embodiments, due to the restriction systems which are active in the microorganism to be transformed, it is necessary to methylate the nucleic acid to be introduced into the microorganism. This can be done using a variety of techniques, including those described below, and further exemplified in the Examples section herein after.

By way of example, in one embodiment, a recombinant microorganism of the disclosure is produced by a method comprises the following steps: introduction into a shuttle microorganism of (i) of an expression construct/vector as described herein and (ii) a methylation construct/vector comprising a methyltransferase gene; expression of the methyltransferase gene; isolation of one or more constructs/vectors from the shuttle microorganism; and, introduction of the one or more construct/vector into a destination microorganism.

In one embodiment, the methyltransferase gene of step B is expressed constitutively. In another embodiment, expression of the methyltransferase gene of step B is induced.

The shuttle microorganism is a microorganism, preferably a restriction negative microorganism that facilitates the methylation of the nucleic acid sequences that make up the expression construct/vector. In a particular embodiment, the shuttle microorganism is a restriction negative E. coli, Bacillus subtilis, or Lactococcus lactis.

The methylation construct/vector comprises a nucleic acid sequence encoding a methyltransferase.

Once the expression construct/vector and the methylation construct/vector are introduced into the shuttle microorganism, the methyltransferase gene present on the methylation construct/vector is induced. Induction may be by any suitable promoter system although in one particular embodiment of the disclosure, the methylation construct/vector comprises an inducible lac promoter and is induced by addition of lactose or an analogue thereof, more preferably isopropyl-β-D-thio-galactoside (IPTG). Other suitable promoters include the ara, tet, or T7 system. In a further embodiment of the disclosure, the methylation construct/vector promoter is a constitutive promoter.

In a particular embodiment, the methylation construct/vector has an origin of replication specific to the identity of the shuttle microorganism so that any genes present on the methylation construct/vector are expressed in the shuttle microorganism. Preferably, the expression construct/vector has an origin of replication specific to the identity of the destination microorganism so that any genes present on the expression construct/vector are expressed in the destination microorganism.

Expression of the methyltransferase enzyme results in methylation of the genes present on the expression construct/vector. The expression construct/vector may then be isolated from the shuttle microorganism according to any one of a number of known methods. By way of example only, the methodology described in the Examples section described hereinafter may be used to isolate the expression construct/vector.

In one particular embodiment, both construct/vector are concurrently isolated.

The expression construct/vector may be introduced into the destination microorganism using any number of known methods. However, by way of example, the methodology described in the Examples section hereinafter may be used. Since the expression construct/vector is methylated, the nucleic acid sequences present on the expression construct/vector are able to be incorporated into the destination microorganism and successfully expressed.

It is envisaged that a methyltransferase gene may be introduced into a shuttle microorganism and over-expressed. Thus, in one embodiment, the resulting methyltransferase enzyme may be collected using known methods and used in vitro to methylate an expression plasmid. The expression construct/vector may then be introduced into the destination microorganism for expression. In another embodiment, the methyltransferase gene is introduced into the genome of the shuttle microorganism followed by introduction of the expression construct/vector into the shuttle microorganism, isolation of one or more constructs/vectors from the shuttle microorganism and then introduction of the expression construct/vector into the destination microorganism.

It is envisaged that the expression construct/vector and the methylation construct/vector as defined above may be combined to provide a composition of matter. Such a composition has particular utility in circumventing restriction barrier mechanisms to produce the recombinant microorganisms of the disclosure.

In one particular embodiment, the expression construct/vector and/or the methylation construct/vector are plasmids.

Persons of ordinary skill in the art will appreciate a number of suitable methyltransferases of use in producing the microorganisms of the disclosure. However, by way of example the Bacillus subtilis phage ΦT1 methyltransferase and the methyltransferase described in the Examples herein after may be used. Nucleic acids encoding suitable methyltransferases will be readily appreciated having regard to the sequence of the desired methyltransferase and the genetic code.

Any number of constructs/vectors adapted to allow expression of a methyltransferase gene may be used to generate the methylation construct/vector.

In one embodiment, the substrate comprises CO. In one embodiment, the substrate comprises CO₂ and CO. In another embodiment, the substrate comprises CO₂ and H₂. In another embodiment, the substrate comprises CO₂ and CO and H₂.

In one particular embodiment of the disclosure, the gaseous substrate fermented by the microorganism is a gaseous substrate containing CO. The gaseous substrate may be a CO-containing waste gas obtained as a by-product of an industrial process, or from some other source such as from automobile exhaust fumes. In certain embodiments, the industrial process is selected from the group consisting of ferrous metal products manufacturing, such as a steel mill, non-ferrous products manufacturing, petroleum refining processes, gasification of coal, electric power production, carbon black production, ammonia production, methanol production and coke manufacturing. In these embodiments, the CO-containing gas may be captured from the industrial process before it is emitted into the atmosphere, using any convenient method. The CO may be a component of syngas (gas comprising carbon monoxide and hydrogen). The CO produced from industrial processes is normally flared off to produce CO₂ and therefore the disclosure has particular utility in reducing CO₂ greenhouse gas emissions and producing butanol for use as a biofuel. Depending on the composition of the gaseous CO-containing substrate, it may also be desirable to treat it to remove any undesired impurities, such as dust particles before introducing it to the fermentation. For example, the gaseous substrate may be filtered or scrubbed using known methods.

In particular embodiments of the disclosure, the gaseous substrate fermented by the microorganisms a gaseous substrate comprising CO2 and H2. The CO2/H2 containing substrate may be a waste gas obtained as a by-product of an industrial process. In certain embodiments the industrial process is selected from the group consisting of hydrogen production. In certain embodiments the gaseous substrate comprising CO2 and H2 may be a blended gas stream, wherein at least a portion of the gas stream is derived from one or more industrial process is blended with at least a portion of CO2 or H2 to optimise the CO2:H2 ratio of the gaseous substrate. This may be particularly beneficial for industrial gas streams rich in either CO2 or H2. Examples of industrial process which produce by-product gas streams which can be used as a source for a CO2 and H2 substrate, or a CO2 and H2 blended substrate include coke manufacturing, refinery processes, ammonia production processes, methanol production processes, acetic acid production, natural gas refineries and power plants.

It will be appreciated that for growth of the bacteria and conversion of gas to products comprising, for example, alcohols to occur, a suitable liquid nutrient medium in addition to the CO— and/or CO₂-containing substrate gas will need to be fed to the bioreactor. The substrate and media may be fed to the bioreactor in a continuous, batch or batch fed fashion. A nutrient medium will contain vitamins and minerals sufficient to permit growth of the micro-organism used. Anaerobic media suitable for fermentation to produce one or more products using CO and/or CO₂ are known in the art. For example, suitable media are described Biebel (2001). In one embodiment of the disclosure the media is as described in the Examples section herein after.

The fermentation should desirably be carried out under appropriate conditions for the fermentation supporting the conversion of the gas to products comprising alcohols to occur. Reaction conditions that should be considered include pressure, temperature, gas flow rate, liquid flow rate, media pH, media redox potential, agitation rate (if using a continuous stirred tank reactor), inoculum level, maximum gas substrate concentrations to ensure that CO and/or CO₂ in the liquid phase does not become limiting, and maximum product concentrations to avoid product inhibition.

In addition, it is often desirable to increase the CO and/or CO₂ concentration of a substrate stream (or CO and/or CO₂ partial pressure in a gaseous substrate) and thus increase the efficiency of fermentation reactions where CO and/or CO₂ is a substrate. Operating at increased pressures allows a significant increase in the rate of CO and/or CO₂ transfer from the gas phase to the liquid phase where it can be taken up by the micro-organism as a carbon source to make products comprising alcohols. This in turn means that the retention time (defined as the liquid volume in the bioreactor divided by the input gas flow rate) can be reduced when bioreactors are maintained at elevated pressure rather than atmospheric pressure. The optimum reaction conditions will depend partly on the particular micro-organism of the disclosure used. However, in general, it is preferred that the fermentation be performed at pressure higher than ambient pressure. Also, since a given CO— and/or CO₂-to-at least alcohol conversion rate is in part a function of the substrate retention time, and achieving a desired retention time in turn dictates the required volume of a bioreactor, the use of pressurized systems can greatly reduce the volume of the bioreactor required, and consequently the capital cost of the fermentation equipment. According to examples given in U.S. Pat. No. 5,593,886, reactor volume can be reduced in linear proportion to increases in reactor operating pressure, i.e. bioreactors operated at 10 atmospheres of pressure need only be one tenth the volume of those operated at 1 atmosphere of pressure.

By way of example, the benefits of conducting a gas-to-ethanol fermentation at elevated pressures has been described. For example, WO 02/08438 describes gas-to-ethanol fermentations performed under pressures of 30 psig and 75 psig, giving ethanol productivities of 150 g/l/day and 369 g/l/day respectively. However, example fermentations performed using similar media and input gas compositions at atmospheric pressure were found to produce between 10 and 20 times less ethanol per litre per day.

It is also desirable that the rate of introduction of the CO and/or CO₂-containing gaseous substrate is such as to ensure that the concentration of CO and/or CO₂ in the liquid phase does not become limiting. This is because a consequence of CO— and/or CO₂-limited conditions may be that one or more product is consumed by the culture.

The composition of gas streams used to feed a fermentation reaction can have a significant impact on the efficiency and/or costs of that reaction. For example, O₂ may reduce the efficiency of an anaerobic fermentation process. Processing of unwanted or unnecessary gases in stages of a fermentation process before or after fermentation can increase the burden on such stages (to products comprising where the gas stream is compressed before entering a bioreactor, unnecessary energy may be used to compress gases that are not needed in the fermentation). Accordingly, it may be desirable to treat substrate streams, particularly substrate streams derived from industrial sources, to remove unwanted components and increase the concentration of desirable components.

In certain embodiments a culture of a bacterium of the disclosure is maintained in an aqueous culture medium. Preferably the aqueous culture medium is a minimal anaerobic microbial growth medium. Suitable media are known in the art and described for example in U.S. Pat. Nos. 5,173,429 and 5,593,886 and WO 02/08438, and as described in the Examples section herein after.

Alcohols, or a mixed stream containing alcohols and/or one or more other products, may be recovered from the fermentation broth by methods known in the art, such as fractional distillation or evaporation, pervaporation, gas stripping and extractive fermentation, including for example, liquid-liquid extraction.

In certain embodiments of the disclosure, alcohols and one or more products are recovered from the fermentation broth by continuously removing a portion of the broth from the bioreactor, separating microbial cells from the broth (conveniently by filtration), and recovering one or more products from the broth. Alcohols may conveniently be recovered for example by distillation. Acetone may be recovered for example by distillation. Any acids produced may be recovered for example by adsorption on activated charcoal. The separated microbial cells are preferably returned to the fermentation bioreactor. The cell free permeate remaining after any alcohol(s) and acid(s) have been removed is also preferably returned to the fermentation bioreactor. Additional nutrients (such as B vitamins) may be added to the cell free permeate to replenish the nutrient medium before it is returned to the bioreactor.

Also, if the pH of the broth was adjusted as described above to enhance adsorption of acetic acid to the activated charcoal, the pH should be re-adjusted to a similar pH to that of the broth in the fermentation bioreactor, before being returned to the bioreactor.

Products may be recovered following fermentation using any appropriate methodology including but not limited to pervaporation, reverse osmosis, and liquid extraction techniques.

One embodiment is directed to a genetically engineered microorganism capable of producing a product from a gaseous substrate, wherein the microorganism comprises an iterative pathway comprising:

a) a nucleic acid encoding a group of enzymes that are capable of catalyzing the conversion of (Cₙ)-acyl CoA to 3-ketoacyl-CoA;
b) a nucleic acid encoding a group of exogenous enzymes capable of catalyzing the conversion of 3-ketoacyl-CoA to β-hydroxyacyl-CoA;
c) a nucleic acid encoding a group of exogenous enzymes capable of catalyzing the conversion of 3-hydroxyacyl-CoA to trans-Δ²-Enoyl-CoA;
d) a nucleic acid encoding a group of exogenous enzymes capable of catalyzing the conversion of trans-Δ²-Enoyl-CoA to (Cₙ₊₂) acyl-CoA;
e) one or more termination enzymes; and wherein the microorganism is a C1-fixing bacteria comprising a disruptive mutation in a thioesterase.

The microorganism according to an embodiment, wherein the iterative pathway is a β-oxidation pathway in a reverse biosynthetic direction.

The microorganism according to an embodiment, wherein the nucleic acid encoding a group of enzymes that are capable of catalyzing the conversion of (Cₙ)-acyl CoA to 3-ketoacyl-CoA of a) is a thiolase, an acyl-CoA acetyltransferase, or a polyketide synthase.

The microorganism according to an embodiment, wherein the nucleic acid encoding a group of enzymes that are capable of catalyzing the conversion of β-ketoacyl-CoA to β-hydroxyacyl-CoA of b) is a β-Ketoacyl-CoA reductase or a β-hydroxyacyl-CoA dehydrogenase.

The microorganism according to an embodiment, wherein the nucleic acid encoding a group of exogenous enzymes capable of catalyzing the conversion of β-ketoacyl-CoA to trans-Δ²-Enoyl-CoA of c) is a β-hydroxyacyl-CoA dehydratase.

The microorganism according to an embodiment, wherein the nucleic acid encoding a group of exogenous enzymes capable of catalyzing the conversion of trans-Δ²-Enoyl-CoA to (Cₙ₊₂) acyl-CoA of d) is a trans-Enoyl-CoA reductase or butyryl-CoA dehydrogenase/electron transferring flavoprotein AB (Bcd-EtfAB).

The microorganism according to an embodiment, wherein the one or more termination enzymes are selected from alcohol-forming coenzyme-A thioester reductase, an aldehyde-forming CoA thioester reductase, an alcohol dehydrogenase, a thioesterase, an acyl-CoA:acetyl-CoA transferase, a phosphotransacylase and a carboxylate kinase; aldehyde ferredoxin oxidoreductase; an aldehyde-forming CoA thioester reductase, an aldehyde decarbonylase, alcohol dehydrogenase; aldehyde dehydrogenase, and an acyl-CoA reductase.

The microorganism according to an embodiment, wherein the exogenous enzymes enable production of Cₙ₊₂ Acetoacid, Cₙ₊₂ 3-OH-acid, Cₙ₊₂ Enoate, Cₙ₊₂ 1-acid, Cₙ₊₂ ketone, Cₙ₊₂ methyl-2-ol, Cₙ₊₂ 1,3-diol, 1,4-diol, 1,6-diol, Cₙ₊₂ 2-en-1-ol, Cₙ₊₂ 1-alcohol, diacids, or any combination thereof.

The microorganism according to an embodiment, wherein the microorganism is a member of a genus selected from the group consisting of Acetobacterium, Alkalibaculum, Blautia, Butyribacterium, Clostridium, Eubacterium, Moorella, Oxobacter, Sporomusa, and Thermoanaerobacter.

The microorganism according to an embodiment, wherein the group exogenous enzymes selected from a), b), c), d), and e), are arranged in a single operon in any order, or in multiple operons in any order.

The microorganism according to an embodiment, which are selected from Clostridium autoethanogenum, Clostridium ljungdahlii, Clostridium ragsdalei, Escherichia coli, Saccharomyces cerevisiae, Clostridium acetobutylicum, Clostridium beijerinckii, Clostridium saccharbutyricum, Clostridium saccharoperbutylacetonicum, Clostridium butyricum, Clostridium diolis, Clostridium kluyveri, Clostridium pasteurianum, Clostridium novyi, Clostridium difficile, Clostridium thermocellum, Clostridium cellulolyticum, Clostridium cellulovorans, Clostridium phytofermentans, Lactococcus lactis, Bacillus subtilis, Bacillus licheniformis, Zymomonas mobilis, Klebsiella oxytoca, Klebsiella pneumonia, Corynebacterium glutamicum, Trichoderma reesei, Cupriavidus necator, Pseudomonas putida, Lactobacillus plantarum, or Methylobacterium extorquens.

The bacteria according to an embodiment, wherein the microorganism further comprises a disruptive mutation in a primary-secondary alcohol dehydrogenase gene, a 3-hydroxybutyryl CoA dehydrogenase gene, phosphate acetyltransferase (pta), acetate kinase (ack), aldehyde-alcohol dehydrogenase (adhE1), beta-hydroxybutyrate dehydrogenase (bdh), ctf, or any combination thereof.

The microorganism according to an embodiment, wherein the product is selected from primary alcohols, trans Δ² fatty alcohols, β-keto alcohols, 1,3-diols, 1,4-diols, 1,6-diols, diacids, β-hydroxy acids, carboxylic acids, or hydrocarbons.

The microorganism according to an embodiment, further comprising an acyl-CoA primer and extender, wherein the primer and extender are capable of cyclic, iterative pathway operation.

The microorganism according to an embodiment, wherein primer and extender is selected from oxalyl-CoA, acetyl-CoA, malonyl CoA, succinyl-CoA, hydoxyacetyl-CoA, 3-hydroxyproprionyl-CoA, 4-hydroxybutyryl-CoA, 2-aminoacetyl-CoA, 3-aminopropionyl-CoA, 4-aminobutyryl-CoA, isobutyryl-CoA, 3-methyl-butyryl-CoA, 2-hydroxyproprionyl-CoA, 3-hydroxybutyryl-CoA, 2-aminoproprionyl-CoA, propionyl-CoA, and valeryl-CoA.

The microorganism according to an embodiment, wherein the primer and/or extender is acetyl-CoA.

The microorganism according to an embodiment, wherein the microorganism further comprises a disruptive mutation in more than one thioesterase.

The microorganism according to an embodiment, wherein the group of enzymes of a), b), c), d), and e) are non-native to the microorganism.

One embodiment is a method of producing a product, the method comprising culturing the engineered microorganism of claim 1, in the presence of a gaseous substrate.

The method according to an embodiment, wherein the gaseous substrate comprises a C1-carbon source comprising CO, CO₂, and/or H₂.

The method according to an embodiment, wherein the product is selected from primary alcohols, 1,4-diols, 1,6-diols, diacids, trans Δ² fatty alcohols, β-keto alcohols, 1,3-diols, β-hydroxy acids, carboxylic acids, or hydrocarbon.

### EXAMPLES

The following examples further illustrate the methods and compositions of the disclosure but should not be construed to limit its scope in any way.

In this work, modular expression of rBOX pathway genes, specifically thiolase (THL), β-ketoacyl-CoA reductase (KCR), β-hydroxyl-CoA dehydratase (HCD), enoyl-CoA reductase/butyryl-CoA dehydrogenase, electron transferring protein AB complex (bcd-etfAB), and termination enzymes (thioesterase, acyl-CoA reductase, phosphate transacetylase, carboxylate kinase) was carried out in C1-fixing bacteria to produce C4-C10 alcohols and acids. The rBOX pathway and genes that were heterologously expressed in the following examples are shown in Table 2.

TABLE 2
rBOX pathway gene variants used for pathway prototyping

| Enzyme | Gene | Source |
|---|---|---|
| Thiolase | fadA | Escherichia coli |
| | Cace_th1A | Clostridium acetobutylicum |
| | Cklu_th1A | Clostridium kluyveri |
| | Eco_thlA | Escherichia coli |
| | Reut_bktB | Ralstonia eutropha |
| | Reut_Bktb_M158A | Ralstonia eutropha |
| | atoB | Escherichia coli |
| β-ketoacyl-CoA reductase | Cklu_Hbd1 | Clostridium kluyveri DSM555 |
| | Cklu_Hbd2 | Clostridium kluyveri DSM556 |
| | Cace_Hbd1 | Clostridium acetobutylicum |
| | Cnec_PhaB | Cupriavidus necator |
| | Cbei_Hbd | Clostridium beijerinckii |
| | Cace_Hbd2 | Clostridium acetobutylicum |
| | | ATCC824 |
| | DJ012_hbd | Clostridium beijerinckii |
| | DJ019_hbd | Clostridium beijerinckii |
| β-hydroxyacyl-CoA | Cac_Crt | Clostridium acetobutylicum |
| dehydratase | | |
| | DJ012_FabZ | Clostridium beijerinckii DJ012 |
| | Ctyr_crt | Clostridium tyrobutyricum |
| | Cbut_crt | Clostridium butyricum |
| | Pae_phaJ1 | Pseudomonas aeruginosa |
| | Pae_phaJ4 | Pseudomonas aeruginosa |
| | Acav_phaJ | Aeromonas caviae |
| | Ec_FadB | Escherichia coli |
| enoyl-CoA | Cac_bcd_etfAB | Clostridium acetobutylicum |
| reductase/butyryl-CoA | | ATCC824 |
| dehydrogenase, electron | | |
| transferring protein AB | | |
| complex | | |
| | Cbei-bcd_etfAB | Clostridium beijerinckii NCIMB |
| | | 8052 |
| | Cty_bcd_etfAB | Clostridium tyrobutyricum |
| | Tde_ter | Treponema denticola |
| | Fib_tre | Fibrobacter succinogenes |
| | Eug_ter | Euglena gracilis |
| | Ilo_ter | Idiomarina loihiensis |
| phosphate transacetylase | Ca_Ptb | Clostridium acetobutylicum |
| | | ATCC824 |
| | Cu_Ptb | Clostridium butyricum |
| | Bli_Ptb | Bacillus licheniformis |
| carboxylate kinase | Ca_Buk | Clostridium acetobutylicum |
| | | ATCC824 |
| | Cu_Buk | Clostridium butyricum |
| | Bli_Buk | Bacillus licheniformis |
| Thioesterase | CAETHG_1524 | Clostridium autoethanogenum |
| | CAETHG_0718 | Clostridium autoethanogenum |
| | CAETHG_1780 | Clostridium autoethanogenum |
| | Eco_TesB | E. coli |
| | Ppu-TesB | Pseudomonas putida |
| | Fsu-TE2108 | Fibrobacter succinogenes S85 |
| | Pru-TE655 | Prevotella ruminicola |
| | Pru-TE1687 | Prevotella ruminicola |
| | Cpa-TE | Cuphea palustris |
| | Uca-TE | Umbellularia californicaTE |
| | CpFatB1.2-M4 | Cuphea palustris |
| | Aba- | Acinetobacter baylyi |
| | TEG17RA165R | |
| | Te1 | Escherichia coli |
| | Te2 | Escherichia coli |
| Acyl-CoA reductase | Cace_AdhE2 | Clostridium acetobutylicum |
| | Cbei_Ald | Clostridium beijerinckii |
| | Maqu2507 | Marinobacter aquaeolei |
| | Aca_acr1 | Acinetobacter baylyi |
| | DJ006_acr1 | Clostridium |
| | | saccharoperbutylacetonicum N1- |
| | | 4(HMT) |
| | DJ008_acr1 | Clostridium beijerinckii |
| | DJ052_acr1 | Clostridium beijerinckii |
| | DJ079_acr1 | Clostridium beijerinckii |
| | DJ322_acr1 | Clostridium beijerinckii |

### Example 1. Proof-of-Concept of rBOX Pathway in C. autoethanogenum

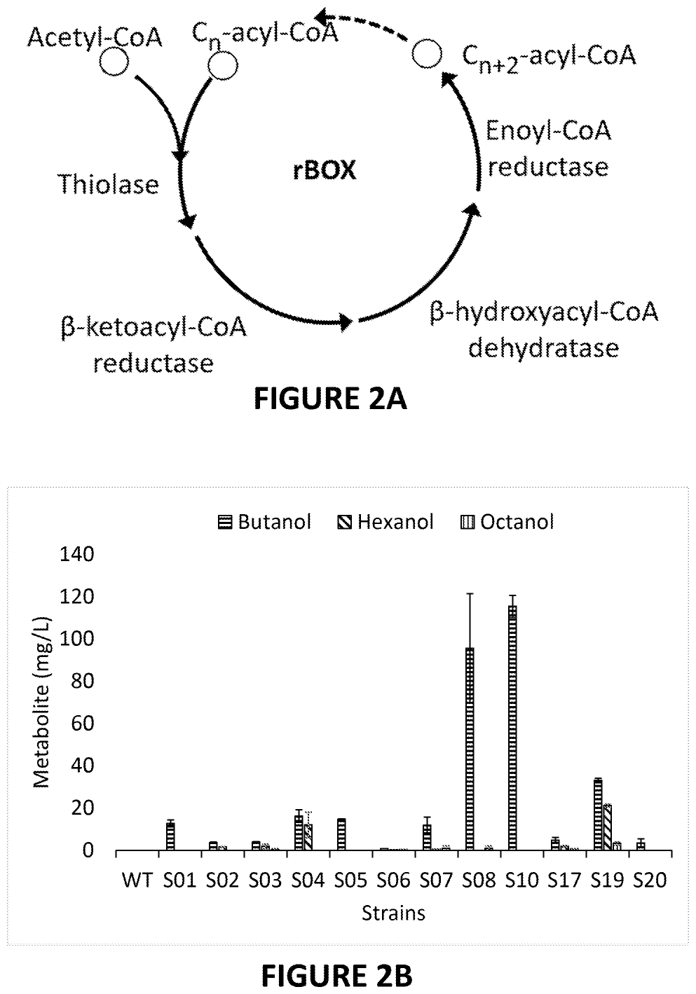

To determine whether a fully functional rBOX pathway can be expressed in C. autoethanogenum, combinations of four genes expressing thiolase (THL), β-ketoacyl-CoA reductase (KCR), β-hydroxyl-CoA dehydratase (HCD), enoyl-CoA reductase/butyryl-CoA dehydrogenase, electron transferring protein AB complex (BCD-ETFAB) were selected (Table 3, FIG. 2A). Three genes (THL, HCD, BCD-ETFAB) were assembled in Clostridium-E. coli shuttle vector pMTL8315 (Heap, J Microbiol Methods, 78: 79-85, 2009). These shuttle vectors have a pre-cloned clostridial promoter and terminator. Each of the genes was flanked with a promoter and a terminator. KCR was cloned into Clostridium-E. coli shuttle vector pMTL8225 (Heap, J Microbiol Methods, 78: 79-85, 2009). Both these plasmids were transformed into C. autoethanogenum strain with thioesterase knockout (CAETHG_1780). Resulting strains were confirmed via colony PCR for the presence of both the plasmids and all four rBOX pathway genes. Each strain was subjected to autotrophic growth in 250 ml Schott bottles with 10 mL minimal media in the presence of 150 kPa synthetic gas mix (50% CO, 10% H₂, 30% CO₂, and 10% N₂) at 37° C. for 14 days with regular samplings for biomass measurement and C4-C8 alcohol analysis. Butanol was detected in all twelve strains with S08 and S10 making 96 mg/L and 115 mg/L of butanol respectively (FIG. 2B). Hexanol was observed in 7 strains, with S19 achieving the highest titers (21 mg/L) (FIG. 2B). Trace amounts (>1 mg/L) of octanol were obtained in S07, S08 and S19 (FIG. 2B). No butanol, hexanol, or octanol was detected in the control (WT) strain that did not harbor any of the rBOX pathway genes (FIG. 2B).

TABLE 3
First round (R1a) of rBOX strains with three or four genes expressed.

| | | | | β-ketoacyl- | | | | |
|---|---|---|---|---|---|---|---|---|
| Design | Promoter | | Promoter | CoA | Promoter | β-hydroxyacyl- | Promoter | Butyryl-CoA |
| # | 1 | Thiolase | 2 | reductase | 3 | dehydrogenase | 4 | dehydrogenase |
| S01 | P2 | ThlA1 | P1 | Hbd1 | P1 | Crt1 | P1 | Bcd_etfAB1 |
| S02 | P2 | ThlA2 | P1 | Hbd1 | P1 | Crt1 | P1 | Bcd_etfAB1 |
| S03 | P2 | ThlA3 | P1 | Hbd1 | P1 | Crt1 | P1 | Bcd_etfAB1 |
| S04 | P2 | ThlA4 | P1 | Hbd1 | P1 | Crt1 | P1 | Bcd_etfAB1 |
| S05 | P2 | ThlA1 | P1 | Hbd2 | P1 | Crt1 | P1 | Bcd_etfAB1 |
| S06 | P2 | ThlA1 | P1 | Hbd3 | P1 | Crt2 | P1 | Bcd_etfAB1 |
| S07 | P2 | ThlA1 | P1 | Hbd3 | P1 | Crt3 | P1 | Bcd_etfAB1 |
| S08 | P2 | ThlA1 | P1 | Hbd4 | | | P1 | Bcd_etfAB1 |
| S10 | P2 | ThlA1 | P1 | Hbd1 | P1 | Crt1 | P1 | Bcd_etfAB3 |
| S17 | P2 | ThlA3 | P1 | Hbd4 | P1 | | P1 | Bcd_etfAB1 |
| S19 | P2 | ThlA3 | P1 | Hbd4 | | | P1 | Bcd_etfAB3 |
| S20 | P2 | ThlA3 | P1 | Hbd3 | P1 | Crt2 | P1 | Bcd_etfAB3 |

### Example 2. Heterologous Expression of Termination Enzymes to Improve Medium Chain Alcohol Production

To determine the impact of heterologous expression of termination enzymes, such as thioesterase, or phosphotransacylase/carboxylate kinase, or acyl-CoA reductase, on pathway flux towards targeted products, a new set of strains was designed with termination enzymes included building upon strain S01 (Table 4).

TABLE 4
rBOX strains with termination enzymes expressed in addition to the core rBOX pathway enzymes used in strain S01.

| | | | | β-ketoacyl- | | | | | | |
|---|---|---|---|---|---|---|---|---|---|---|
| Design | Promoter | | Promoter | CoA | Promoter | β-hydroxyacyl- | Promoter | Butyryl-CoA | Promoter | Termination |
| # | 1 | Thiolase | 2 | reductase | 3 | dehydrogenase | 4 | dehydrogenase | 5 | enzyme |
| S11 | P2 | ThlA1 | P1 | Hbd1 | P1 | Crt1 | P1 | Bcd_etfAB1 | P2 | Ptb1, Buk2 |
| S12 | P2 | ThlA1 | P1 | Hbd1 | P1 | Crt1 | P1 | Bcd_etfAB1 | P1 | Ptb1 Buk2 |
| S13 | P2 | ThlA1 | P1 | Hbd1 | P1 | Crt1 | P1 | Bcd_etfAB1 | P1 | Acr2 |
| S15 | P2 | ThlA1 | P1 | Hbd1 | P1 | Crt1 | P1 | Bcd_etfAB1 | P1 | Te1 |
| S16 | P2 | ThlA1 | P1 | Hbd1 | P1 | Crt1 | P1 | Bcd_etfAB1 | P1 | Te2 |

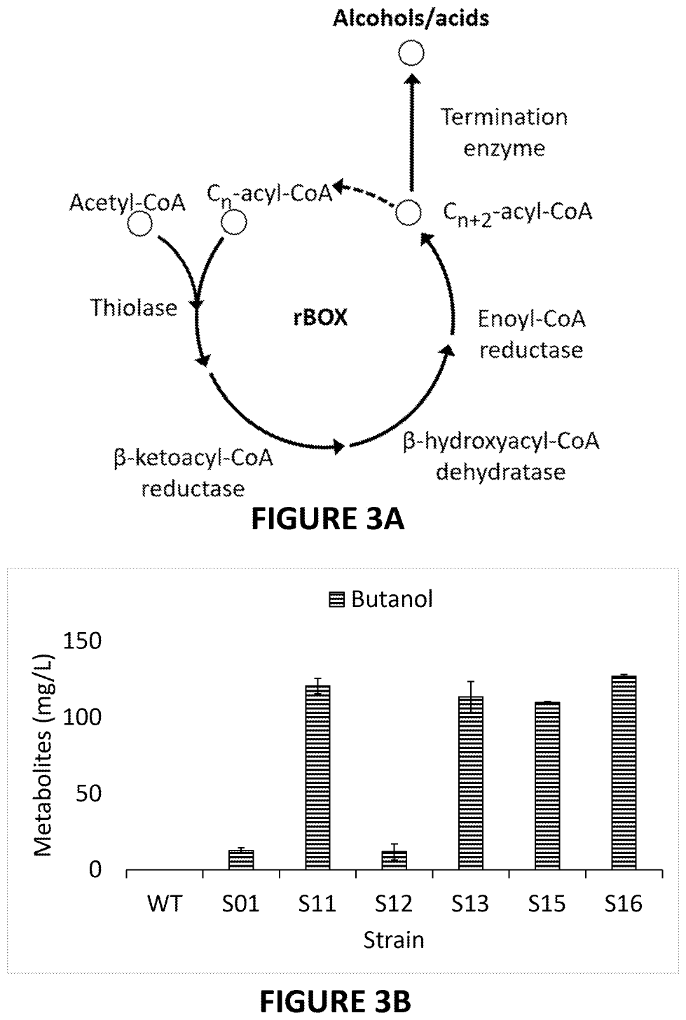

Genes encoding these termination enzymes (TE) were cloned into Clostridium-E. coli shuttle vector pMTL8225 (Heap, J Microbiol Methods, 78: 79-85, 2009) that also had a KCR. Plasmid pMTL8315 containing THL, HCD, BCD-ETFAB, and plasmid 8225 containing TE and KCR were transformed into C. autoethanogenum strain with thioesterase knockout (CAETHG_1780). S01 was used as a control. Resulting strains S11, S12, S13, S15, and S16 had the same four genes as S01, and had an additional termination enzyme (FIG. 3A). These strains were confirmed via colony PCR for the presence of both the plasmids and all the cloned rBOX pathway genes. Each strain was subjected to autotrophic growth in 250 ml Schott bottles with 10 mL minimal media in the presence of 150 kPa synthetic gas mix (50% CO, 10% H₂, 30% CO₂, and 10% N2) at 37° C. for 14 days with regular samplings for biomass measurement and C4-C8 alcohol analysis.

With the exception of S12, all strains with termination enzyme expressed (S11, S13, S15, and S16) showed at least 9-fold improvement in butanol production compared to the control strain, S01 (FIG. 3B). Neither hexanol nor octanol was detected in these strains indicating that the thiolase used is not conducive to hexanol or octanol production (FIG. 3B). These strains also produced butyric acid, ranging between 67-111 mg/L (data not shown).

### Example 3. Expression of Core rBOX Enzymes Variants and Termination Enzyme Variants to Improve Hexanol to Butanol Ratio

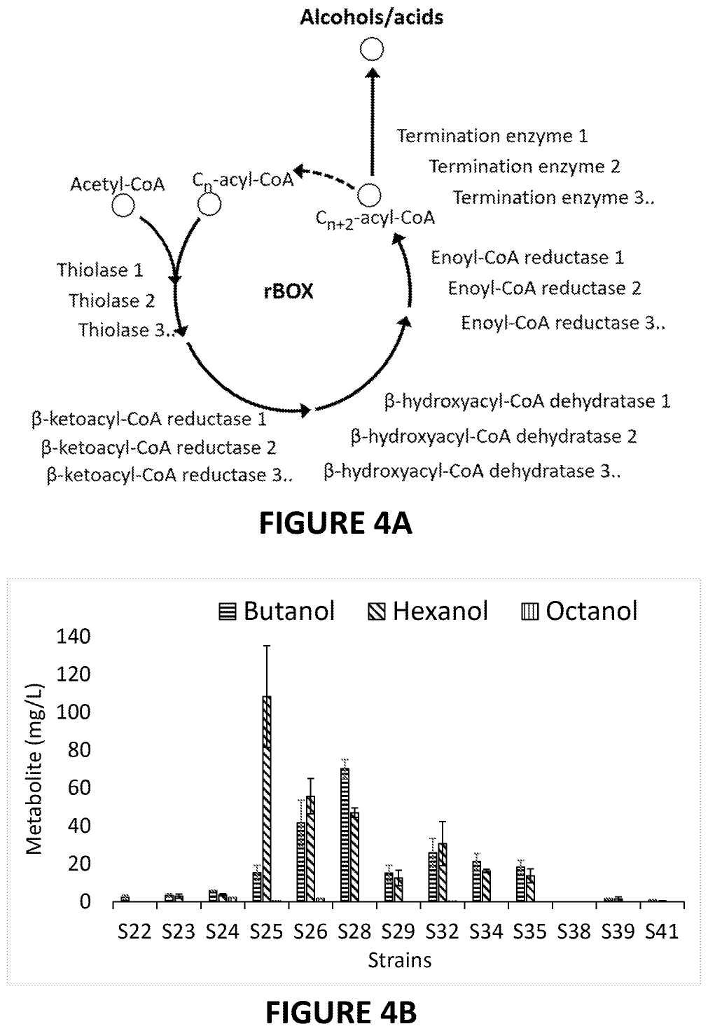

New set of rBOX strains were designed with varying homologs of rBOX core enzymes combined with termination enzyme variants with the objective of improving hexanol to butanol ratio (FIG. 4A, Table 5).

TABLE 5
Second round of rBOX strains with varying homologs of termination
enzymes in addition to variants of core rBOX pathway enzymes.

| Strain | Promoter | Gene | Promoter | Gene | Promoter | Gene | Promoter | Gene | Promoter | Gene |
|---|---|---|---|---|---|---|---|---|---|---|
| number | 1 | 1 | 2 | 2 | 3 | 3 | 4 | 4 | 5 | 5 |
| S21 | P2 | ThlA1 | P1 | Hbd1 | P1 | Crt2 | P1 | Bcd_etfAB1 | P1 | Te1 |
| S22 | P2 | ThlA2 | P1 | Hbd3 | P1 | Crt2 | P1 | Bcd_etfAB1 | P1 | Te2 |
| S23 | P2 | ThlA2 | P1 | Hbd3 | P1 | Crt4 | P1 | Bcd_etfAB1 | P1 | Te2 |
| S24 | P2 | ThlA2 | P1 | Hbd1 | P1 | Crt1 | P1 | Bcd_etfAB1 | P1 | Ptb1, |
| | | | | | | | | | | Buk2 |
| S25 | P2 | ThlA2 | P1 | Hbd1 | P1 | Crt5 | P1 | Bcd_etfAB1 | P1 | Te2 |
| S26 | P2 | ThlA2 | P1 | Hbd1 | P1 | Crt1 | P1 | Bcd_etfAB1 | P1 | Acr2 |
| S28 | P2 | ThlA2 | P1 | Hbd1 | P1 | Crt1 | P1 | Bcd_etfAB1 | P1 | Te1 |
| S29 | P2 | ThlA2 | P1 | Hbd1 | P1 | Crt1 | P1 | Bcd_etfAB1 | P1 | Te2 |
| S32 | P2 | ThlA3 | P1 | Hbd1 | P1 | Crt1 | P1 | Bcd_etfAB1 | P1 | Te2 |
| S34 | P2 | ThlA4 | P1 | Hbd1 | P1 | Crt1 | P1 | Bcd_etfAB1 | P1 | Te1 |
| S35 | P2 | ThlA4 | P1 | Hbd1 | P1 | Crt1 | P1 | Bcd_etfAB1 | P1 | Te2 |
| S38 | P1 | ThlA2 | P1 | Hbd1 | P1 | Crt1 | P1 | Bcd_etfAB1 | P1 | Te1/Te2 |
| S39 | P1 | ThlA2 | P1 | Hbd1 | P1 | Crt1 | P3 | Bcd_etfAB1 | P1 | Te1/Te2 |
| S41 | P2 | ThlA4 | P1 | Hbd1 | P1 | Crt1 | P1 | Bcd_etfAB3 | P1 | Te2 |

Resulting strains S21-S41 were confirmed via colony PCR for the presence of both the plasmids and all the cloned rBOX pathway genes. Each strain was subjected to autotrophic growth in 250 ml Schott bottles with 10 mL minimal media in the presence of 150 kPa synthetic gas mix (50% CO, 10% H₂, 30% CO₂, and 10% N2) at 37° C. for 14 days with regular samplings for biomass measurement and C4-C8 alcohol analysis.

Of all the strains tested, S25 produced the highest level of hexanol, reaching 108 mg/L hexanol (FIG. 4B). This strain also had the highest selectivity to hexanol compared to all other strains with hexanol to butanol ratio being 7:1. Approximately 2 mg/L of octanol was produced by S25 (FIG. 4B).

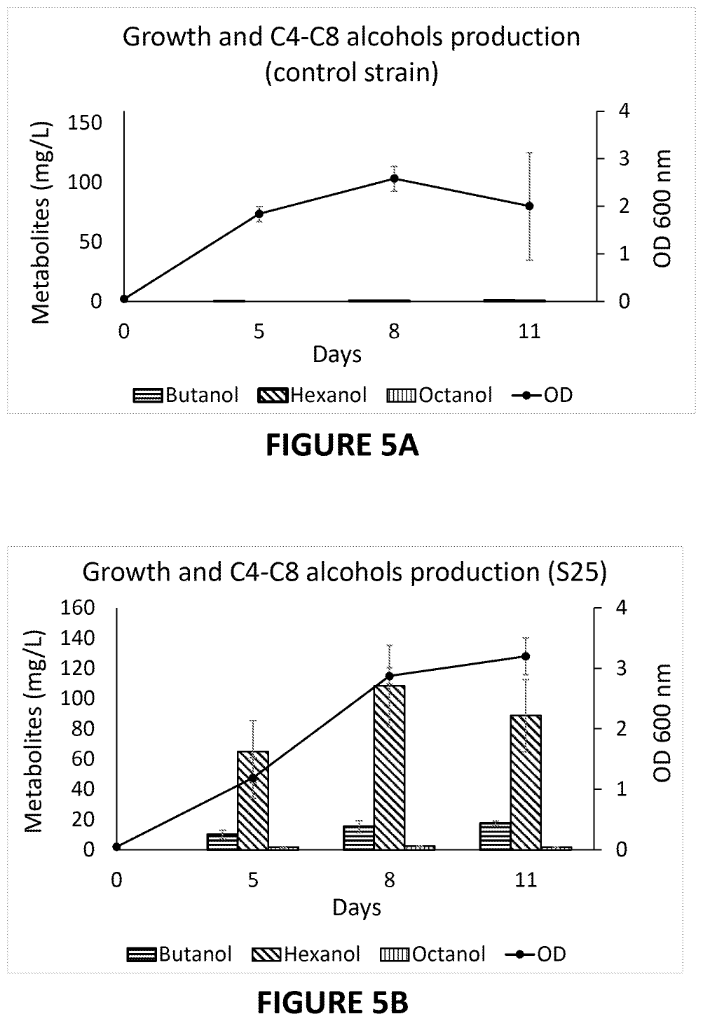

Growth and metabolite profile of S25 was monitored for 11 days in comparison to the chassis strain that did not express rBOX pathway genes (FIG. 5A). Peak hexanol titer was achieved at day 8 (FIG. 5B).

### Example 4: Medium Chain Alcohol Production from Syngas Fermentation in 1.5 L CSTR

Strain S25 was characterized in 1.5 L CSTR in batch mode to determine whether high hexanol selectivity observed in Schott bottles is obtainable in CSTR also. Actively growing (early exponential) culture from Schott bottles was used to inoculate 1.5 L CSTR with a synthetic gas blend (50% CO, 10% H₂, 30% CO₂, and 10% N2).

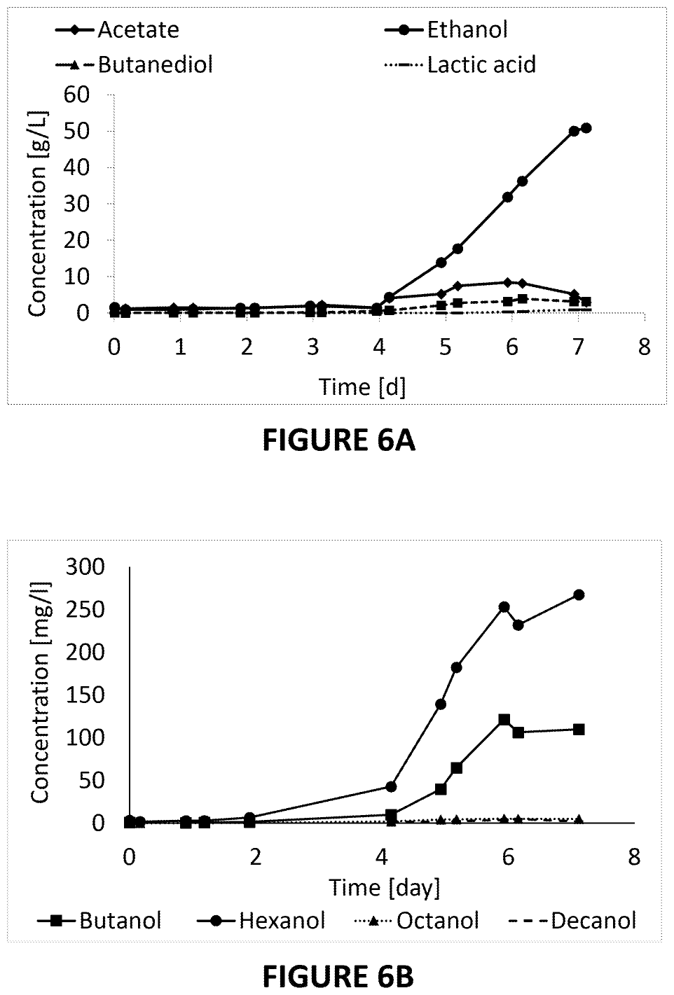

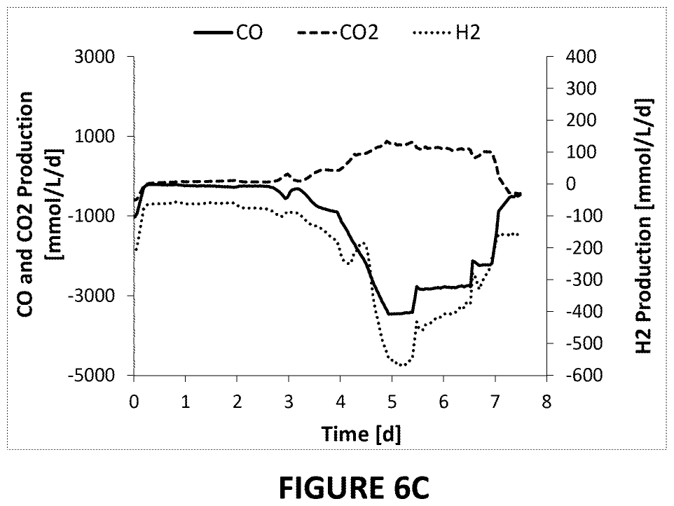

Strain S25 achieved a peak biomass concentration of 3.93 gDCW/L (FIG. 6A) and reached a peak CO uptake of 3454 mmol/L/d (FIG. 6C). In addition to a peak ethanol concentration of 50 g/L (FIG. 6A), this strain produced a peak hexanol titer of 267 mg/L (FIG. 6B) and peak butanol titer of 109 mg/L (FIG. 6B). This strain also produced −5 mg/L of octanol and decanol (FIG. 6B).

### Example 5: Acid to Alcohol Conversion in CSTR Runs

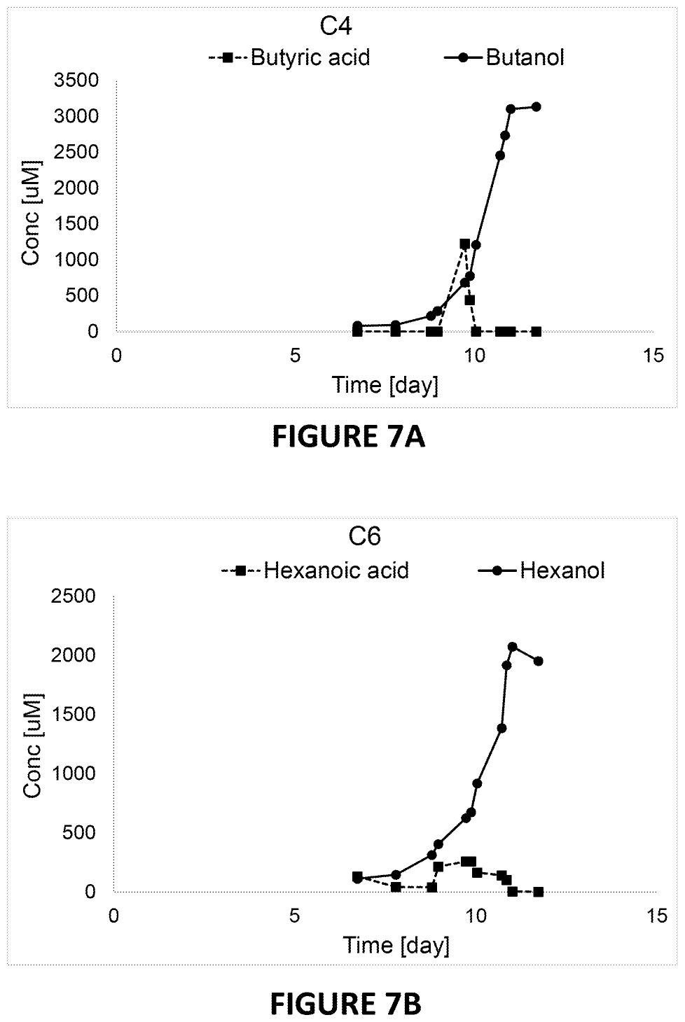

Acid to alcohol conversion rate, specifically for butyric acid and hexanoic acid was measured for strain S32 during its characterization in 1.5 L CSTR (batch mode). A high acid to alcohol conversion rate was observed for C4 (FIG. 7A) and C6 products (FIG. 7B).

### Example 6: Improvement in Hexanol Selectivity Via Rearrangement of Genes on Plasmids

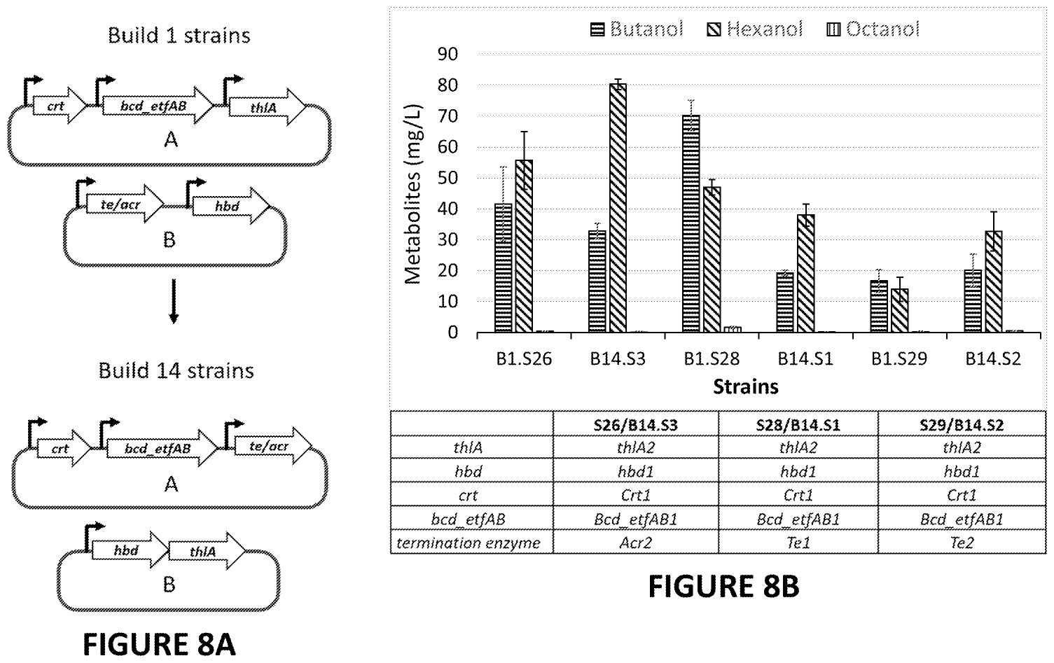

The order of genes on pMTL8225 and pMTL8315 plasmids was rearranged with expression of THL and KCR in a single operon. (FIG. 8A). Strains constructed using this new plasmid architecture exhibited higher hexanol to butanol ratios compared to strains containing THL and KCR in separate operons (FIG. 8B).

In an embodiment, Ptb-Buk has been demonstrated in above examples for several different products, but can be extended to further products, for example production of 2-buten-1-ol, 3-methyl-2-butanol, 1,3-hexanediol (HDO). 2-Buten-1-ol can be produced via Ptb-Buk, AOR and an alcohol dehydrogenase from crotonyl-CoA. 1,3-Hexanediol can be produced via Ptb-Buk, AOR and an alcohol dehydrogenase from 3-hydroxy-hexanoyl-CoA. By combining Ptb-Buk, Adc and an alcohol dehydrogenase (such as native primary:secondary alcohol dehydrogenase), 3-methyl-2-butanol can be formed from acetobutyryl-CoA.

All of the precursors, crotonyl-CoA, 3-hydroxy-hexanoyl-CoA, or acetobutyryl-CoA can be formed by reduction and elongation of acetyl-CoA, acetoacetyl-CoA and 3-HB-CoA which are described in previous examples via known fermentation pathways of, for example, Clostridium kluyveri (Barker, PNAS USA, 31: 373-381, 1945; Seedorf, PNAS USA, 105: 2128-2133, 2008) and other Clostridia. Involved enzymes include crotonyl-CoA hydratase (crotonase) or crotonyl-CoA reductase, butyryl-CoA dehydrogenase or trans-2-enoyl-CoA reductase, thiolase or acyl-CoA acetyltransferase and 3-hydroxybutyryl-CoA dehydrogenase or acetoacetyl-CoA hydratase. Respective genes from C. kluyveri or other Clostridia have been cloned on an expression plasmid (U.S. 2011/0236941) and and then transformed as described previously in C. autoethanogenum strains pta-ack::ptb-buk or CAETHG_1524:: ptb-buk from previous examples for production of 2-buten-1-ol, 3-methyl-2-butanol, 1,3-hexanediol (HDO). 2-Buten-1-ol, 3-methyl-2-butanol, and 1,3-hexanediol (HDO) may be precursors for further downstream products.

While these are only a few examples, it should be clear that this pathway can be further extended using the same enzymes or engineered variants thereof that have specificity for higher chain length to produce a range of C4, C6, C8, C10, C12, C14 alcohols, ketones, enols or diols. Different type of molecules can be obtained also by using primer or extender units different than acetyl-CoA in the thiolase step as been described elsewhere (Cheong, Nature Biotechnol, 34: 556-561, 2016).

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavor in any country.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. The term “consisting essentially of” limits the scope of a composition, process, or method to the specified materials or steps, or to those that do not materially affect the basic and novel characteristics of the composition, process, or method. The use of the alternative (e.g., “or”) should be understood to mean either one, both, or any combination thereof of the alternatives. As used herein, the term “about” means±20% of the indicated range, value, or structure, unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein. Variations of those embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A genetically engineered microorganism capable of producing a product from a gaseous substrate, wherein the microorganism comprises an iterative pathway comprising:
a) a nucleic acid encoding a group of enzymes that are capable of catalyzing the conversion of (Cₙ)-acyl CoA to β-ketoacyl-CoA;
b) a nucleic acid encoding a group of exogenous enzymes capable of catalyzing the conversion of β-ketoacyl-CoA to β-hydroxyacyl-CoA;
c) a nucleic acid encoding a group of exogenous enzymes capable of catalyzing the conversion of β-hydroxyacyl-CoA to trans-Δ²-Enoyl-CoA;
d) a nucleic acid encoding a group of exogenous enzymes capable of catalyzing the conversion of trans-Δ²-Enoyl-CoA to (Cₙ₊₂) acyl-CoA;
e) one or more termination enzymes; and
wherein the microorganism is a C1-fixing bacteria comprising a disruptive mutation in a thioesterase.

2. The microorganism of claim 1, wherein the iterative pathway is a β-oxidation pathway in a reverse biosynthetic direction.

3. The microorganism of claim 1, wherein the nucleic acid encoding a group of enzymes that are capable of catalyzing the conversion of (Cₙ)-acyl CoA to β-ketoacyl-CoA of a) is a thiolase, an acyl-CoA acetyltransferase, or a polyketide synthase.

4. The microorganism of claim 1, wherein the nucleic acid encoding a group of enzymes that are capable of catalyzing the conversion of β-ketoacyl-CoA to β-hydroxyacyl-CoA of b) is a β-Ketoacyl-CoA reductase or a β-hydroxyacyl-CoA dehydrogenase.

5. The microorganism of claim 1, wherein the nucleic acid encoding a group of exogenous enzymes capable of catalyzing the conversion of β-ketoacyl-CoA to trans-Δ²-Enoyl-CoA of c) is a β-hydroxyacyl-CoA dehydratase.

6. The microorganism of claim 1, wherein the nucleic acid encoding a group of exogenous enzymes capable of catalyzing the conversion of trans-Δ²-Enoyl-CoA to (Cₙ₊₂) acyl-CoA of d) is a trans-Enoyl-CoA reductase or butyryl-CoA dehydrogenase/electron transferring flavoprotein AB (Bcd-EtfAB).

7. The microorganism of claim 1, wherein the one or more termination enzymes are selected from alcohol-forming coenzyme-A thioester reductase, an aldehyde-forming CoA thioester reductase, an alcohol dehydrogenase, a thioesterase, an acyl-CoA:acetyl-CoA transferase, a phosphotransacylase and a carboxylate kinase; aldehyde ferredoxin oxidoreductase; an aldehyde-forming CoA thioester reductase, an aldehyde decarbonylase, alcohol dehydrogenase; aldehyde dehydrogenase, and an acyl-CoA reductase.

8. The microorganism of claim 1, wherein the group of exogenous enzymes selected from a), b), c), d), and e), are arranged in a single operon in any order, or in multiple operons in any order.

9. The microorganism of claim 1, wherein the exogenous enzymes enable production of Cₙ₊₂ Acetoacid, Cₙ₊₂ 3-OH-acid, Cₙ₊₂ Enoate, Cₙ₊₂ 1-acid, Cₙ₊₂ ketone, Cₙ₊₂ methyl-2-ol, Cₙ₊₂ 1,3-diol, 1,4-diol, 1,6-diol, Cₙ₊₂ 2-en-1-ol, Cₙ₊₂ 1-alcohol, diacids or any combination thereof.

10. The microorganism of claim 1, which are selected from Clostridium autoethanogenum, Clostridium ljungdahlii, Clostridium ragsdalei, Escherichia coli, Saccharomyces cerevisiae, Clostridium acetobutylicum, Clostridium beijerinckii, Clostridium saccharobutyricum, Clostridium saccharoperbutylacetonicum, Clostridium butyricum, Clostridium diolis, Clostridium kluyveri, Clostridium pasteurianum, Clostridium novyi, Clostridium difficile, Clostridium thermocellum, Clostridium cellulolyticum, Clostridium cellulovorans, Clostridium phytofermentans, Lactococcus lactis, Bacillus subtilis, Bacillus licheniformis, Zymomonas mobilis, Klebsiella oxytoca, Klebsiella pneumoniae, Corynebacterium glutamicum, Trichoderma reesei, Cupriavidus necator, Pseudomonas putida, Lactobacillus plantarum, or Methylobacterium extorquens.

11. The microorganism of claim 1, wherein the microorganism further comprises a disruptive mutation in a primary-secondary alcohol dehydrogenase gene, a 3-hydroxybutyryl CoA dehydrogenase gene, a phosphate acetyltransferase (pta), an acetate kinase (ack), an aldehyde-alcohol dehydrogenase (adhE1), a beta-hydroxybutyrate dehydrogenase (bdh), a CoA transferase (ctf), or any combination thereof.

12. The microorganism of claim 1, wherein the product is selected from (Cₙ)-alcohols, primary alcohols, trans Δ² fatty alcohols, β-keto alcohols, 1,3-diols, 1,4-diols, 1,6-diols, diacids, β-hydroxy acids, β-ketoacids carboxylic acids, fatty acids, fatty acid methyl esters, ketoacids, hydrocarbons, or any combination thereof.

13. The microorganism of claim 1, further comprising an acyl-CoA primer and extender, wherein the primer and extender are capable of cyclic, iterative pathway operation.

14. The microorganism of claim 13, wherein primer and extender is selected from oxalyl-CoA, acetyl-CoA, malonyl CoA, succinyl-CoA, hydoxyacetyl-CoA, 3-hydroxyproprionyl-CoA, 4-hydroxybutyryl-CoA, 2-aminoacetyl-CoA, 3-aminopropionyl-CoA, 4-aminobutyryl-CoA, isobutyryl-CoA, 3-methyl-butyryl-CoA, 2-hydroxyproprionyl-CoA, 3-hydroxybutyryl-CoA, 2-aminoproprionyl-CoA, propionyl-CoA, butyryl-CoA, and valeryl-CoA.

15. The microorganism of claim 13, wherein the primer and/or extender is acetyl-CoA.

16. The microorganism of claim 1, wherein the microorganism further comprises a disruptive mutation in more than one thioesterase.

17. The microorganism of claim 1, wherein the group of enzymes of a), b), c), d), and/or e) are non-native to the microorganism.

18. A method of producing a product, the method comprising culturing the engineered microorganism of claim 1, in the presence of a gaseous substrate.

19. The method of claim 18, wherein the gaseous substrate comprises a C1-carbon source comprising CO, CO₂, and/or H₂.

20. The method of claim 18, wherein the product is selected from (Cₙ)-alcohols, primary alcohols, trans Δ² fatty alcohols, β-keto alcohols, 1,3-diols, 1,4-diols, 1,6-diols, diacids, β-hydroxy acids, β-ketoacids carboxylic acids, fatty acids, fatty acid methyl esters, ketoacids, hydrocarbons, or any combination thereof.
